SEQUENCE LISTING (68 sequences)

SEQ ID NO: 1 - DNA, 804 bp, Cryptococcus neoformans
  source 1..804: mol_type = genomic DNA
  CDS 1..804
atgtctgacgacggacaagtacaaaggggaaaggccggcattagctggccggctcaagaactaacctcggatcccattgccaaattcttccagtacggctctaaactctcatggcactggaactggaccaagcattggaagggccctctggtgccggagacctctgacgaccttgagattgacgcagaatttgtgcccatgatctggtcaccccaatctttagatgatggttgcgacttgcaagaaggatggaatcttctcttgggtttcaacgagcctgacctcgacaacgaagctgttgcaagccatcgctctccgcaggaagctgcagacgcgtggatccagctggcacaactccgtaccgatccagacaaccagcacctcgtttcccccgctgtagcatccaacgtggaatggcttaaagagttcctctccctgattccagaagacacttatcccgcctacttggctgtgcacctctacacaaccacttttgatgattttgtcggcaagatggagatgtaccacaacgagtttggattgcctattatcttgactgaattctgcatgcagagttgggacgaaggtgttccaggcccagaggaccagcagcaagtccatgattacatgggccaaacaacaaaatggcttgatgaaactgactatgttattaagtactgttggtttggcgctgttcgtgatacggcgaacttgcacgacgtccaccccttcaaccgactcatggatgaaaacggcgagattaccccattgggtttccaatacatgtatggtgggcatgagtaa

SEQ ID NO: 2 - protein, 267 aa, Cryptococcus neoformans
MSDDGQVQRGKAGISWPAQELTSDPIAKFFQYGSKLSWHWNWTKHWKGPLVPETSDDLEIDAEFVPMIWSPQSLDDGCDLQEGWNLLLGFNEPDLDNEAVASHRSPQEAADAWIQLAQLRTDPDNQHLVSPAVASNVEWLKEFLSLIPEDTYPAYLAVHLYTTTFDDFVGKMEMYHNEFGLPIILTEFCMQSWDEGVPGPEDQQQVHDYMGQTTKWLDETDYVIKYCWFGAVRDTANLHDVHPFNRLMDENGEITPLGFQYMYGGHE

SEQ ID NO: 3 - DNA, 1821 bp, Cryptococcus neoformans
  source 1..1821: mol_type = genomic DNA
  CDS 1..1821
atgtcgacccttcccatttatcaccccgttccaacggacgagaaacacccaatatctgccactttggtagacggcgagtttgaccctcgctacattcatcccgccgcaatcggctctcaatacctttatattggcggtccccgcagcgcctatcaggccgcgaaggacaagtacgctggcttgtccaaagtcaagaaaggtctcctcgctcttgccgttgtttggttcggtcttgtcgttggccatcaggctgcgcgtcttgctggcggcaaatgccaccaggacgctcatcatgctcccgccgaatttggcgtgaagcagtggagagaccactcatctcatcgatttggtggccctatcttcctcgaggatggtccacttgactgtcatggtggccgtaaagaccgtgctcctgaggagctttcttccgttgccactgtctacgagtccatcaacgttgtcgggagcaacgatgctaccgacattctctccgccaacgcctctttccctctcaaacttggccgtggcaagcactttgatctcaccttccaaggtgagggtaacgtcatcatctcgagggctgaggaggagtctgaagactctactgtcaacgtttttgttgagtctacttggtccggtgaggaggctgaaggggtcaagatgttgtctggaaaacactctcacgctctctctgttgcttcttctcaatcctcgtctcatattgtccaccttgttcttcctgccaacaagaagcgtcttccttccatctctatcttttctaccaaggaccttactcttgatatccatccatctgttcaggacatccacgtgggaaagctctccctcaagtctgagagcggtgatatcaagcttcctaccctcgctgtcaacaagctcgtggctgagaccgtaaccggtgacgtcggcggtaacttcaacgtcagcaactctttcgttgtcaagacagtcacaggtaacattaacgccattgttaacgttgttcctcactccccacctaaggacaagcttaaccttcataacgttgatgccaagcacgagcacaagaagtttgacagccgtcacggagaacacaatcacgagaagaagcacttcggagggcgtttccactctgaagaagagcgaccttccaagtggtctctcaatattttcaagtctaagaaagaggatgagcctgaacaccctcccccccctccggtctttatcggcgctttctccacctctggcaacattcttctcaaggtcttcggttctcccaacgtctctactgatactaatgtcttctcccataccggtgacgtcgacgttacccatgacaagtcattccacggtttgtacgaggtcggcagcttaaagggcacctatgatgttgtcgtgagggacggcaaggtgcatcgagtcctggaggaatacgtcactgaggagggaggcaagcagaagggccttgccttcgttcccaagaacagaaagactgagggctcccacgagaagaggcacttccgcaatgctgaaagcgttgatggcgagcttccccctccccctcctggtaagggccacggtcctgatggtcccgatggtcctgatggtcctggaggtcctagtggtcctggaggtcctggtggtcctgatggtcctggtggtcctggtggtcctggtggccctggaggtcccggtggtcccggtggtcccggtggtcccggccccgaccacccccgtggtcctcctccttgggttgtcttcccccccggtcactcagaagtcttcgtccacactgaagttggcaacgccaagattgtcctctaa

SEQ ID NO: 4 - protein, 606 aa, Cryptococcus neoformans
MSTLPIYHPVPTDEKHPISATLVDGEFDPRYIHPAAIGSQYLYIGGPRSAYQAAKDKYAGLSKVKKGLLALAVVWFGLVVGHQAARLAGGKCHQDAHHAPAEFGVKQWRDHSSHRFGGPIFLEDGPLDCHGGRKDRAPEELSSVATVYESINVVGSNDATDILSANASFPLKLGRGKHFDLTFQGEGNVIISRAEEESEDSTVNVFVESTWSGEEAEGVKMLSGKHSHALSVASSQSSSHIVHLVLPANKKRLPSISIFSTKDLTLDIHPSVQDIHVGKLSLKSESGDIKLPTLAVNKLVAETVTGDVGGNFNVSNSFVVKTVTGNINAIVNVVPHSPPKDKLNLHNVDAKHEHKKFDSRHGEHNHEKKHFGGRFHSEEERPSKWSLNIFKSKKEDEPEHPPPPPVFIGAFSTSGNILLKVFGSPNVSTDTNVFSHTGDVDVTHDKSFHGLYEVGSLKGTYDVVVRDGKVHRVLEEYVTEEGGKQKGLAFVPKNRKTEGSHEKRHFRNAESVDGELPPPPPGKGHGPDGPDGPDGPGGPSGPGGPGGPDGPGGPGGPGGPGGPGGPGGPGGPGPDHPRGPPPWVVFPPGHSEVFVHTEVGNAKIVL

SEQ ID NO: 5 - DNA, 594 bp, Cryptococcus neoformans
  source 1..594: mol_type = genomic DNA
  CDS 1..594
atgcgttttacttctatcatcgttgccgctcttccgcttgtcggctctgtcttcgctgcccccttcgctgagaaggattctatcgcttcttcccccgacttggtcaagaaggaggttaacgtcctctctgtcgtcaatgaagtccagtctagggttaatgctgctgccgccatgccccgccagtctcaagcggatgttgaggcctgtctcaacactgtcattgatgcctttaactggtgcggtggccagctcggtattgacgtttccgccagcgccagcgccaatgccggtgctagcatccattacttgcgtcgtgagattattgcccgtgatgacgacaaggaggctgttgctcaggcactctctagcgttgttcagaccgttaatgtcggcatcgtccagcagatccccagccaattcatcaacatccctggcgtctccaaccttgttaaccagcttgacattgctctcagtctcatccttaagggtgttgacgctattctcgccggtgtcctctacctcgtcaaggcccttctcatcgatgttggcatcatcctcgactcgcttctcggcggtctcctttccatcctttaa

SEQ ID NO: 6 - protein, 197 aa, Cryptococcus neoformans
MRFTSIIVAALPLVGSVFAAPFAEKDSIASSPDLVKKEVNVLSVVNEVQSRVNAAAAMPRQSQADVEACLNTVIDAFNWCGGQLGIDVSASASANAGASIHYLRREIIARDDDKEAVAQALSSVVQTVNVGIVQQIPSQFINIPGVSNLVNQLDIALSLILKGVDAILAGVLYLVKALLIDVGIILDSLLGGLLSIL

SEQ ID NO: 7 - DNA, 894 bp, Cryptococcus neoformans
  source 1..894: mol_type = genomic DNA
  CDS 1..894
atgtctgccgtcgaagcaccctccgcctcgcaggccatctggcccgagctcactgaagaccaccccctttcgcagctcaactctcgcctccctactatcctttcagaggctggtcactcccaaatctggggcgttactcttacttactccactcccccaaccttctctagccttattattctgcaaaaattccttcgttccgtggataataacgtggatgaggctgccacggctctaggcaagacactcaagtggcggaaggactggggattggacgcgcgggcggacaaaaaagagaaggaaaactttgggcccgattttgaaggcttaggatatgtgaccaagatcaagaaaaatgatggcggagatgagatcgtgacttggaacgtttatggagctgtgaaggatttgaaatcgacctttggggatcttgaccgattccttcgatggcgtgtcaatcttatggaggaggctatcgcccatcttcatctcgctaccacctctactcccatcccagactttaacgccggtattgatccccatcgcatggcacaagtccatctatatgaaggtgtctcattccttcgcatggatcctcatgtgaaagctgcctccaaggcaaccattgagcttatggcggccaactatcccgaacttctttctcgcaaattctttgtgggcgtgcctttgataatgagctggatgtttcaggccgtgcgaatgttcgtttccgctgagactgccaagaagtttgtggtcattagctacaaggagaatctggcgaatgagctgggagaacttgaaggtgtgcccaaggagtatggtggaaagggtctcagtttgggcgaacttcagaaccagctgcgaggggaggacgcggtgacttcttcgtaa

SEQ ID NO: 8 - protein, 297 aa, Cryptococcus neoformans
MSAVEAPSASQAIWPELTEDHPLSQLNSRLPTILSEAGHSQIWGVTLTYSTPPTFSSLIILQKFLRSVDNNVDEAATALGKTLKWRKDWGLDARADKKEKENFGPDFEGLGYVTKIKKNDGGDEIVTWNVYGAVKDLKSTFGDLDRFLRWRVNLMEEAIAHLHLATTSTPIPDFNAGIDPHRMAQVHLYEGVSFLRMDPHVKAASKATIELMAANYPELLSRKFFVGVPLIMSWMFQAVRMFVSAETAKKFVVISYKENLANELGELEGVPKEYGGKGLSLGELQNQLRGEDAVTSS

SEQ ID NO: 9 - DNA, 2094 bp, Cryptococcus neoformans
  source 1..2094: mol_type = genomic DNA
  CDS 1..2094
atgtcagagctattcaaggacatcccagagtttgtagagaccgacatcggagagagccttgcagccagaacggaaacccttggctccttcagagaactaggccctccagacctctgccatgttatgaaagtttatgggaaaccgccgactcaacgagagatcgggtcctatcactactgctctggaatagaggcttcgtcctctgcgtcactcgctgcctatctcaactctttgcagttttcagtggaagattcgtctgcatggtttggcaaggggtcggcatggaaagttcgaagcgggacgtattgctgcttcaatgccttttcacgggtagatatgcgggtggaagccaatattcccggcggtgtcgacgcttttgtggttgatcttcacggtcaaagacaccctgcgacccccgagctctggcaagagacgtacctgtctgcgatcctgcgtgctattagatatgcggacgatgcctcctataggttggcagggtatagaaagctggatccgatcacaacgccagaagcagaggaaagattcctcaaagccgccgaagcgctgttcttcaagggctggcagcttggctcagatcccgaaatacaggtcgccacagttgtcaccaaccacctgacctctgccattcttaaatacttttccgactctttcagacttcatcgagccgccaaccttttcgaaaggatgatggacaaggagccagaggtagccgctctagtggcgaagagttacatcggcatgaacgaggagatcaaagctgtcaagatcatgaacgctgcccttgccgccaatcctcaatcctatcccatccttcatgcccaagtcgatttcctcctttccaagcacaaatacgaatgggcccagcaagtcgcccagcaggcggtcaattctgcacccagcgagttcacgacttgggccaaactcacggagacgtacatcgagttggggcaactcgaccaggctttgttgacactcaactcatgtccaatgtttacttataacgaaagagatctccatcggatgcctacccctgcaaagtccaatatgccagtcaagaagtttatcgcagactccaatttggtggatgaagattcgtcacgagagaacgaggccgatatcgctctcctccgtctccccgctcccaacctccgcggcacattcgccaaagcgtactccctcctcactctccttgtctctaagattggttgggatgagcttctcaaaattagatcctccgtcttcgtcatggaagaggaatatcggctgcataaaacgaacgtttctgttgatatgaatggcgaagcgggtgacggcgcgtccattgcgggcttgaagaggacctcgtccgaagaggtcaacactcccagcgatatacctaccatcaggatatcaagcgagtcgatgcgcactcctaatacggctccaggaccagggttcagcgaaaaggcaagtactcacaagcccgctctggagaagcccgagacagcacaagcgaatgaagatcccaattcgcctttggggatgaagagtgaaggggaacagccggtttcggcgttttctcataagcgattatgtgagagatggttagataacctctttttagttctgtatgaagacttgagagtctacaccatttggagagcagagatatctcatttcaaaacccagcacatgtcataccgaaagactggtaccgagtgggagatccttggtgaacttgccacacgtttgcatcacaaagaagaagccaaggacgcgtaccaacgctgtctcgactccaaattcagcgcaaaagcacttatgaagcttcttgaaacgtatgcgaatgagggcgatcttcaaaagaccttgacggcggctgtgaggctgacaacctatcaccatcgatggtatatggacgcgtcatacccgtccatggtcgcgcattatttgtacaaggtcggactcatacatggacatgccaaattacaatacacaatgctcagtatgaacctgccggtcgggatctttgaaataatgcaaggctatatgaaatacggggcgacgttcaacgtcgaaggttcagaattctag

SEQ ID NO: 10 - protein, 697 aa, Cryptococcus neoformans
MSELFKDIPEFVETDIGESLAARTETLGSFRELGPPDLCHVMKVYGKPPTQREIGSYHYCSGIEASSSASLAAYLNSLQFSVEDSSAWFGKGSAWKVRSGTYCCFNAFSRVDMRVEANIPGGVDAFVVDLHGQRHPATPELWQETYLSAILRAIRYADDASYRLAGYRKLDPITTPEAEERFLKAAEALFFKGWQLGSDPEIQVATVVTNHLTSAILKYFSDSFRLHRAANLFERMMDKEPEVAALVAKSYIGMNEEIKAVKIMNAALAANPQSYPILHAQVDFLLSKHKYEWAQQVAQQAVNSAPSEFTTWAKLTETYIELGQLDQALLTLNSCPMFTYNERDLHRMPTPAKSNMPVKKFIADSNLVDEDSSRENEADIALLRLPAPNLRGTFAKAYSLLTLLVSKIGWDELLKIRSSVFVMEEEYRLHKTNVSVDMNGEAGDGASIAGLKRTSSEEVNTPSDIPTIRISSESMRTPNTAPGPGFSEKASTHKPALEKPETAQANEDPNSPLGMKSEGEQPVSAFSHKRLCERWLDNLFLVLYEDLRVYTIWRAEISHFKTQHMSYRKTGTEWEILGELATRLHHKEEAKDAYQRCLDSKFSAKALMKLLETYANEGDLQKTLTAAVRLTTYHHRWYMDASYPSMVAHYLYKVGLIHGHAKLQYTMLSMNLPVGIFEIMQGYMKYGATFNVEGSEF

SEQ ID NO: 11 - DNA, 1662 bp, Cryptococcus neoformans
  source 1..1662: mol_type = genomic DNA
  CDS 1..1662
atgtcgttagcggacgcccttctggcagaccttgatggtctctcggatgacgaagctcgatctccttctcctggccccgaggcctcctcctcgtcaatgccgcctcctggtttgcccaacaaaggaaaacgtcccgccagcgctatggaagtcgatgatggcgaaggaggtgcgaatgaagatgagggagacgatatgaagctggaagacgggacgagtgctgtgggatttgtacctgaaggaggtgtaaggcctgcagatgagctggacaaggaggaagtggaaaaaaccgatatgaagggtgtcgaggatgtgaagaaagtagccaggttggcaggaagccagaagcttcgagatgttctggcagatatcataaaatacaccgagtctcccaccgatatgtcttcgtctgccggtcccctcgaggagaatccagagtaccatcttgttgtcactgcgaacaacatgtccgtcgaggttgacaacgagattctcatcgtgcacaaattcattcgtgaccactatgctcctcgatttccggaactcgaacagctcattgccgaaccttggacatacattgccgccgttaatgccatcggtcagtctgaagatctaacgaaggtcacattccccaacaccctccctgcggctactgtactctctatcactcttactgctacgacttcccgtggtcggccgctcacgcctgcagagtgggaaacaattcagcgcgccatcgctgtcgcccaaaatctccgttcggcgcgagaacaaattttttcctacgtcgagtcccgtatggctgctgtagcacctaatttgtctgctattgtgggcaccggtatcgctgccaaattacttggtttagcaggtggtctccatgcgtttagtcgacagccgagttgtaatgtgatgctttttggcgcgatgaagaagactttggccacctctcatctttctgctgcctctcagcaacgacataccggctttatcttccaaagctctatagtacagagtgcccagcctgaagatcgaagaagagctcagcgagcggtgtctgccaagtgtgctcttgcggccaggatcgatgcaggaaaggggtctagggacggatcttatggaagaaagtgtttggcggatttgcaaaagaggattgaaaagatggcggaacctcctcccaacaagatgatcaaggcgttgcctatccctcaggagactaacaggaagaagcgtggtggtaagagagctcgaaaagccaaggaagcgtacgcccagaccgaattgagaaagttacaaaaccgaatggagtttggcaaggcggaagaagagatcggggtggacgacgagactgttggtttgggtatgatcggttccgccggaagggtccgaggcgagatggcagatgcgaggagtaaagctaaactttctcgagccaacaaacttcgaactcagctccttggtcgctcagtcacatccaacgacgctgccagcggtatggccacctccttatcattcacgcctgtccaaggtcttgaaatagttacaccctccctctctgcagcccagaaagtacaggctgcgaatgacagatggttctccgggggtacatttacgcatgtaaggaaggggggaagcagtattccgggacaggaacagaaatag

SEQ ID NO: 12 - protein, 553 aa, Cryptococcus neoformans
MSLADALLADLDGLSDDEARSPSPGPEASSSSMPPPGLPNKGKRPASAMEVDDGEGGANEDEGDDMKLEDGTSAVGFVPEGGVRPADELDKEEVEKTDMKGVEDVKKVARLAGSQKLRDVLADIIKYTESPTDMSSSAGPLEENPEYHLVVTANNMSVEVDNEILIVHKFIRDHYAPRFPELEQLIAEPWTYIAAVNAIGQSEDLTKVTFPNTLPAATVLSITLTATTSRGRPLTPAEWETIQRAIAVAQNLRSAREQIFSYVESRMAAVAPNLSAIVGTGIAAKLLGLAGGLHAFSRQPSCNVMLFGAMKKTLATSHLSAASQQRHTGFIFQSSIVQSAQPEDRRRAQRAVSAKCALAARIDAGKGSRDGSYGRKCLADLQKRIEKMAEPPPNKMIKALPIPQETNRKKRGGKRARKAKEAYAQTELRKLQNRMEFGKAEEEIGVDDETVGLGMIGSAGRVRGEMADARSKAKLSRANKLRTQLLGRSVTSNDAASGMATSLSFTPVQGLEIVTPSLSAAQKVQAANDRWFSGGTFTHVRKGGSSIPGQEQK

SEQ ID NO: 13 - DNA, 759 bp, Cryptococcus neoformans
  source 1..759: mol_type = genomic DNA
  CDS 1..759
atgtcatcaactgatctcggaggccaagctgccgtcatcactggcggtggtaagaatcttggtgctttgattgcgaagactctcgccaagcagggagtcaacgttgcgatccattacaactcggccagttccaagtccgagacagaagctacattgaagacactcggatcgtatggggtcaaagccgctgctttccaggccaatcttaccactgaggcatcagttgagaaactcttctcagacgcagcagctgctcttggagtgtccaagttcgatatcgccatcaatacggtcggtaaggttcttaaaaagcctatcgttgaaacaacagagcaaggattcgacgacatgttcctagtcaactcaaagtgtgccttcttttttatcaagcatgcggccaagaatctcaacgaggggggcacgattatatcactcgtgacttcactccttggagcatttgcgcctggttattcaacttatcaaggcagtaaagctcctgtagagtggttcactaagtcggctgccaaggagcttcagcctaagaatattagggtcaactgtgtggctccggggccaatggacactcccttcttttacgggcaagagactgaagatgccgttgctttccataaaagccaggcgctcacaggacggctcacagatattaaagatattgcaccattggtggagttcctttgcaaggataagtggattaccggacaagtcatcttctcaaatggaggttacacgactcgctga

SEQ ID NO: 14 - protein, 252 aa, Cryptococcus neoformans
MSSTDLGGQAAVITGGGKNLGALIAKTLAKQGVNVAIHYNSASSKSETEATLKTLGSYGVKAAAFQANLTTEASVEKLFSDAAAALGVSKFDIAINTVGKVLKKPIVETTEQGFDDMFLVNSKCAFFFIKHAAKNLNEGGTIISLVTSLLGAFAPGYSTYQGSKAPVEWFTKSAAKELQPKNIRVNCVAPGPMDTPFFYGQETEDAVAFHKSQALTGRLTDIKDIAPLVEFLCKDKWITGQVIFSNGGYTTR

SEQ ID NO: 15 - DNA, 2199 bp, Cryptococcus neoformans
  source 1..2199: mol_type = genomic DNA
  CDS 1..2199
atgcagcaccaccccgcggtagcagcacagccgggccgcactattgcccctatcccgcaccaccgcccacagcaaccccggatcactccttacacaccaaacgtacgcgacctcaacccaggacctaagaacagactcatcctcgccctccgctccaacatcccctttgaagtcgactgggcgctaccgcagcttgttgtcgcaagtttcgaccagtcggacgggttcaagctcgaggcatggccagacagcatttgcgcgttgaaggaatggccggccaagtggcttgaaggactagaaagggaagctgcagtgtttgagatgaaagctgggcgattggattttgagggggacgagaatgatgaagaggggaggatggcaaagcgcagaaaaagggatctggcgctgggggcggtggtagagtgggagaacgatctcaaggtggaacaacgggcgaccaactctttgctcgtcctcagaaacgcatccttcaacgcacccaacgcaaagatcctctcaagctcaagcttcctcgcttttctagccgatttcttctctttgcctctaccgtttctccagcatctttgcctgagaaccccagagcctatacatcatatcctcatcattgtccagtccatcttcccccatttgcgcgtggacatgccaggtatcgaccgcatcaagcacatctttggcgtcgtcttccctcagctttttgttgatacccgcgatatcgcaatgatgaacaaccttatccctctcatgatgatgggccagacaatccccaataaccaccctcctccgcctgaactcatccctcatcttctccagcttctcgttctccgtccagcaggcccacttctcgatttgactcttgacatcctcatctccctctccacaaatcccatccactcccgtgccatactttctcatacttctttcccgcatcatctcaaatccatcacagccttactcgaacatcaagctcgtccggtggtgaatgcccttgacccaccgccttctacgagagggaaaatggtgcgtaacccagcgggaccgagttgcagagcagaggaacttaatcaaaggcggacgaaggaacgagaggccgcattgggacatatggatcccatggctggaggtagaccggtgtacaatgaggtaggggataagccaccgacatttagtccggcgacgaagaagaggcttttcaggatgaaagaacccgaaaggtctatcgagtggatgcaccaggcattcgtctactcatcgacagcccaagtccttcaagtgacattctggcacgcctaccgagatttcttcaccaacccagcttgcgtagaaccaatgttgagtgcatctgatgtgatcaagaatgtcactgcagctttccctggagcgagcgcaaaagtttggaccgatgcgagtggtgcgcaaaagtttgtgattgctggtgtcgggttcaggaagcgatcagatgacgatgaaaggtttacatgttactggcatgcatgcacccaacggtactcagctaccaaccccgtccaactgctcgaacacattagcaactaccatctccaaaccttttctgcaccccaatgccaatggggctcatgcgatcacaacctctgcacgtactctcatctcctcacccatatccccctcggccagcctccatcctccatctccgtccctgacgccatctcttgccatatcgcagaccatagtagctccgtcttgcagcgcaagatcaccaatcgtaccgtccctcctttatccagcgttcgtctagccgttcagggggcatttacccctgtcgacgctcgtcgacaacctactggcgccgcccttctcgcggcgttacttatccgtaacctcgcccgtaccctccgtgccgagatctcgctcgccgtgcccgaattgtctcatgctcaaacgcaagaaacggcagatgaagctcaagcgagaaaaaaacaccttctcgaagagaggtatggattgccaatcccggattcggtgttgaaagaagaagaagaggagcaggcgaatgtgcagcaaggccaagatttagatatgagtgaggaagagagggagagggcgaaaaaggcgtttgagaatgtggaggagaggattatgaaggtcatgttggagaatgttagtgggataacgcagtatcttggtgatgcgcttgggctgtag

SEQ ID NO: 16 - protein, 732 aa, Cryptococcus neoformans
MQHHPAVAAQPGRTIAPIPHHRPQQPRITPYTPNVRDLNPGPKNRLILALRSNIPFEVDWALPQLVVASFDQSDGFKLEAWPDSICALKEWPAKWLEGLEREAAVFEMKAGRLDFEGDENDEEGRMAKRRKRDLALGAVVEWENDLKVEQRATNSLLVLRNASFNAPNAKILSSSSFLAFLADFFSLPLPFLQHLCLRTPEPIHHILIIVQSIFPHLRVDMPGIDRIKHIFGVVFPQLFVDTRDIAMMNNLIPLMMMGQTIPNNHPPPPELIPHLLQLLVLRPAGPLLDLTLDILISLSTNPIHSRAILSHTSFPHHLKSITALLEHQARPVVNALDPPPSTRGKMVRNPAGPSCRAEELNQRRTKEREAALGHMDPMAGGRPVYNEVGDKPPTFSPATKKRLFRMKEPERSIEWMHQAFVYSSTAQVLQVTFWHAYRDFFTNPACVEPMLSASDVIKNVTAAFPGASAKVWTDASGAQKFVIAGVGFRKRSDDDERFTCYWHACTQRYSATNPVQLLEHISNYHLQTFSAPQCQWGSCDHNLCTYSHLLTHIPLGQPPSSISVPDAISCHIADHSSSVLQRKITNRTVPPLSSVRLAVQGAFTPVDARRQPTGAALLAALLIRNLARTLRAEISLAVPELSHAQTQETADEAQARKKHLLEERYGLPIPDSVLKEEEEEQANVQQGQDLDMSEEERERAKKAFENVEERIMKVMLENVSGITQYLGDALGL

SEQ ID NO: 17 - DNA, 1305 bp, Cryptococcus neoformans
  source 1..1305: mol_type = genomic DNA
  CDS 1..1305
atgagactcacgattattgccccagactcggttcatgagcacgaagtgtccccttccttgctcatccaagacatcatcaacatcgttgaggcaactgccgaccttcccccggctgttattgttctcacaagtgacgccggtacaccactcacggaccccacaagaactctcgaaagctatgggttaaatggagagaccgccaccatcttccttacacctacaggaccacccgtcgcttcttcgtcttccattccattccctgatgcagatgccgacattgaaaggatgcgtttacaagcgctcggaaatccttctttgatgaatgatttgcgtgagcgtgatccggaaacctttgccgctattcaagggggtactcaaagcttcaaaaaagccctccaactggcgcaatcaagacaaagagatgccgaattcgaaaagcaacgccagattgaagcactcaatgccgacccttatgacattgaagctcagaaaaagattgaggaagcaattcggatggaggccgttttggagaatatgcagcacgctatggaatattcccctgagtcgtttggaaacgtgaccatgctgtatatcaatgtggaagtaaatggtcatcctgttaaggcattcgttgattctggtgcacaaacaacgatcatttcccctgaatgtgccgagcaatgtggaatcatgcgcctgcttgatactcgtttcgcgggtatggccgaaggagtaggaacagctcgtatcctcggtcgtatccactctgcccaaattaagctcggctcactctacctcccttgtgcattctccgtcctcgaaggccgttctgtcgacctcttatttggtcttgacatgcttaaacgccatcaatgctgtatcgacctctccacgaacacgctccggataaataacactgaagtaccctttttgtcggagcacgagctgcctgacaaggcgagaagacgtggggaggcgcaagtggccggggaaatgggtgatgcggcagggcaaggcgtgaaagcgggtgtggcgagtccgaagattgggaagaagacgtttccgggagaggggcatgcgcttggtgcgggcagctcgactggaccagggacggctacggggagtgcaagtgcgacaggtgcaaggactggggggactgcaagtgtcccctcgccttcaaataggtggaaagaggacgatattcaaacgcttgtgaacctgggtgcccctcgagcgcaagctatacagctacttgaagcgtcaggtggaaacgtggatgttgctgcttctatgctctttggttag

SEQ ID NO: 18 - protein, 434 aa, Cryptococcus neoformans
MRLTIIAPDSVHEHEVSPSLLIQDIINIVEATADLPPAVIVLTSDAGTPLTDPTRTLESYGLNGETATIFLTPTGPPVASSSSIPFPDADADIERMRLQALGNPSLMNDLRERDPETFAAIQGGTQSFKKALQLAQSRQRDAEFEKQRQIEALNADPYDIEAQKKIEEAIRMEAVLENMQHAMEYSPESFGNVTMLYINVEVNGHPVKAFVDSGAQTTIISPECAEQCGIMRLLDTRFAGMAEGVGTARILGRIHSAQIKLGSLYLPCAFSVLEGRSVDLLFGLDMLKRHQCCIDLSTNTLRINNTEVPFLSEHELPDKARRRGEAQVAGEMGDAAGQGVKAGVASPKIGKKTFPGEGHALGAGSSTGPGTATGSASATGARTGGTASVPSPSNRWKEDDIQTLVNLGAPRAQAIQLLEASGGNVDVAASMLFG

SEQ ID NO: 19 - DNA, 3555 bp, Cryptococcus neoformans
  source 1..3555: mol_type = genomic DNA
  CDS 1..3555
atgtcttcgccagaaccagaggagcctcgcagaggtgcaagggtcaggaagcaggttaataagtttgatgctagccagcagaacgggaggggcaagagaaagcacattgaagacagggaggacgacgaccaggagggtttgataccagacccggaagacgagtctgatcacgaaccaactcccaagaagaagaagccggcggcaccacgaaaatctcgagcttctgcgggtactaccaagaaggacggaccaaagacaaaaacaaagcctgcagctgaaggcgtgagcgaaatcgtagaaaagactgattcgcctttatttaatgctctccagcaacccgatatcgcccttcaacctctgattgatgagtggatcgagacctaccaacaagccgctggtgatgaaatatcagagcagaaatccattcacgaactggttgtcttcttcattcgatgttgcggtatgactaccgagatcgagcaagctgaagcaacggatgacgatggtatccccgatgtcatcgagcgagtgcaggatgaaagcgttcgcgtagcgttggcgacttatcccttaatttccaaagcaaagaattttaagcccttcaagtccaatttgaacgagttcatttcacactttatttcatcgctcgctctcacacctatcctctttcacactgccgacaatactcctcactcatctctgctcatcccacttctcctcaactggctgatgtgtatgtcatcatcaactcttcgacccatccgtcatacctcaacatacgtgacgctcaggatgaactcggctttgtgtgacgttgctgcggatgtgagcaaagacctgagcgttaagcaaaggcagcgagatgcagaagtcagaaaagctggagctacaaatgcagcgcagaagagagtgaaggctgccgaggacagggtcaaggaagtgcaagaaagaaagcaaactttagaagagttgatgcaggagatctttgatgtgatgttcgtccaccgagttcgcgatgccgatcccaacattcgaaccgattgtctgcgtgaattaggtctgtgggccaaaaaacacccagagtactacgtttcgacttcttatctctcctacttcacccgtggctgtaacgatacccacgctcatgcccgacttgagactgtcaaggctcttgccaacctctacatccgagaaacctttatcagtaacgctcgaaccttgacgatgcgtttagcgcctagggtgattgagatggccaccagggatgtggatttgaatgtgagggtagtggctttgcaggtgattacacttatagacaagacgggtattctgcaagacgaggaggacgaggaaagagataaggtggcgaagcttgttttcgaccaggagcctcgaattcgaaaagctgcaggggcgttcatccttggtttgtgggaagagaggaaagaaggcctcaaagcagtctggtcgggtctgagagcgaacaaaaagaagcgtgcagcaaacatcaccgaagacgaaatgtccaactacctcaactggaaatccctcgctgcagttctcctctacacctctaaatccctggacgacgacccttctggacaaccctctgccctcaaaccaagcctactcattccgtctttacccaatacacagatgacaagggcgactgctgccgtcgagtctatcggtgctgagcatgagctgtggaaagactgggagagcttggtggactatcttttggtggatcactcgactaacgaagaagatatgtggctgctccgtgaagatgaggaaactttcatgttgcaggtgcttttggcttgtattaagcgggaagaaaatgaagaggatgagccggataggaccaaaacgttgataaaggttttgcctcggttatttgccaagcatcaggctgatgttggtcgaatgactgggattttatctgttcccggacacatgaagctcagtctctatctcgacatgcgcatgtcctctgcctacgagtccctctgggatgacatcagcaaacagttcctaaaatacacttcccctaccatcctcacagcatccatttctgcgatcagccatctcgtcggcaactcgtccctttcatccatcaatgaaaccaagctttctgagctgcacgagtctctcttcgcttctctaagagatgcgattggctctgaagatgttgcgcttgtcactttggaggacgagcagatcagccagctggaagcaatcatgctgaggataacgttactgcagagaagtatggatttggtagatgtcatggaggatgaggaagggcagcagagtagcggctgggacattatctgtgcgtttgctgataggggcaaattggggtacaaggaggaagctactatggtagactatgctgttcaaatcatcttcctccacatcacttggctcttcaagcggttcaccaaggaagatgcgcaagatgccaccaagattgatctcctttccacccgacgcgataccgcccttcagacatttaaccagcttttcctcggagaaacgaccaataccgccagtgctgtacgacgtcaagccttcatctctttcatcaatacgtacgtattgttcgccaaacgtgcagagggtaggggaggagctccagcgagcgacgtttgttctgtgacgatgccggaagaagtacagcatagactgggaggggcgttccaagcggtgattgagaggtatgcttccgtcgtggagactagatcagcaggacgggaagagagtcagcagccccccgaactcactcctgatgagatgcacgaggatttccagtttttccaactcgtttccgtttttgtcggtgccatccgatgtggtgtcctcgaggttgaacatgccaaggaacctcttgcccattacagtcgttttggtccaacgtacgatgcgatcgtcaagaagctcgttgatgtacttcgagatgagggtatctacaatagggaggcagatgcggtgcagcatgttgcgggaagcgccttgcagcaatcgttcaacatcttcctcgactctgaggaagacgaaccaactgctcctctggcccttgcccgtgttattgcaactgcgttcgtcatccatggttcccaattcgctatcctaagacaattgcatccatctgatgtttgcgatttccacctcgaagcgcttgactttgtttctctcaaagtctcaacgattgtcaaacaagaaggaaatgcaaggaacaaggagcaaaaatccagactaacaaggaaaaagtgggcagtgctcacattcttcaaggtgctcgtccctcttctcgcgcctgtcacaggtagagatgctctcaagatcaaggctcatctcgaagatgtaatcgactcttctggggtgcaactgacaaccaacaagggttgggatggctaccgagcgtacgaaaagagattagtagggatcgcaagcaaggacccgaatgtgaaaatgatggctagcaagaaggttgtagaaagggaggatactgaacagggtgatgaagacaatgtctttgcaaggcaatga

SEQ ID NO: 20 - protein, 1184 aa, Cryptococcus neoformans
MSSPEPEEPRRGARVRKQVNKFDASQQNGRGKRKHIEDREDDDQEGLIPDPEDESDHEPTPKKKKPAAPRKSRASAGTTKKDGPKTKTKPAAEGVSEIVEKTDSPLFNALQQPDIALQPLIDEWIETYQQAAGDEISEQKSIHELVVFFIRCCGMTTEIEQAEATDDDGIPDVIERVQDESVRVALATYPLISKAKNFKPFKSNLNEFISHFISSLALTPILFHTADNTPHSSLLIPLLLNWLMCMSSSTLRPIRHTSTYVTLRMNSALCDVAADVSKDLSVKQRQRDAEVRKAGATNAAQKRVKAAEDRVKEVQERKQTLEELMQEIFDVMFVHRVRDADPNIRTDCLRELGLWAKKHPEYYVSTSYLSYFTRGCNDTHAHARLETVKALANLYIRETFISNARTLTMRLAPRVIEMATRDVDLNVRVVALQVITLIDKTGILQDEEDEERDKVAKLVFDQEPRIRKAAGAFILGLWEERKEGLKAVWSGLRANKKKRAANITEDEMSNYLNWKSLAAVLLYTSKSLDDDPSGQPSALKPSLLIPSLPNTQMTRATAAVESIGAEHELWKDWESLVDYLLVDHSTNEEDMWLLREDEETFMLQVLLACIKREENEEDEPDRTKTLIKVLPRLFAKHQADVGRMTGILSVPGHMKLSLYLDMRMSSAYESLWDDISKQFLKYTSPTILTASISAISHLVGNSSLSSINETKLSELHESLFASLRDAIGSEDVALVTLEDEQISQLEAIMLRITLLQRSMDLVDVMEDEEGQQSSGWDIICAFADRGKLGYKEEATMVDYAVQIIFLHITWLFKRFTKEDAQDATKIDLLSTRRDTALQTFNQLFLGETTNTASAVRRQAFISFINTYVLFAKRAEGRGGAPASDVCSVTMPEEVQHRLGGAFQAVIERYASVVETRSAGREESQQPPELTPDEMHEDFQFFQLVSVFVGAIRCGVLEVEHAKEPLAHYSRFGPTYDAIVKKLVDVLRDEGIYNREADAVQHVAGSALQQSFNIFLDSEEDEPTAPLALARVIATAFVIHGSQFAILRQLHPSDVCDFHLEALDFVSLKVSTIVKQEGNARNKEQKSRLTRKKWAVLTFFKVLVPLLAPVTGRDALKIKAHLEDVIDSSGVQLTTNKGWDGYRAYEKRLVGIASKDPNVKMMASKKVVEREDTEQGDEDNVFARQ

SEQ ID NO: 21 - DNA, 897 bp, Cryptococcus neoformans
  source 1..897: mol_type = genomic DNA
  CDS 1..897
atgaagtactctgctaccgcagtcgccgttatgggtgccctcgccattcaagccaccccaatcaagagagatgcttacaccccaaccgacattgatatcctacagtatgcgttgactctcgagcacctggagaacaacttctactcctgcgccctcaacaacatggacgctcaagcgttcgccgatgccggattcccagcctgggtacggaacaggtttgagcagattgccgctcacgaggcctcccacgtcgccgttctctccgatgccctcggcgctgacgccaccaagccatgcgagtactccttcccatacaccgacgccaaatcgttcaccgctctcgctcaggtcattgagaatgttggtgtttctgcttacctcggtgccgccggtttcatcatggacaagacctacttgaccgttgctggttccattctcaccaccgaggcccgccaccaggcctggatcgcttccgccgttaacaagcagaacccatggtccggcccatacgacactcctctcggtctctccgatgtctactccattgccgctgccttcatcaccagctgtccatcctccaacccaactctcccagtcaaggcattcccagctctcactctctcttgcgactccgccggttcgactgccaccctcaactataccggcgctgattcatccgacacccttattctctactctggcctcacgaccctcgctctccccatcaccgacatgatggtcaccatcccatcctctcttcagggcattgcttacgcagtcgtgtcttcaacgtctaacaccaccatggttgacgactctaacaccattgccggcccagccatcattgaccttcctttcgcttcttccgccagcaaccccaacttcactggtatgtaa

SEQ ID NO: 22 - protein, 298 aa, Cryptococcus neoformans
MKYSATAVAVMGALAIQATPIKRDAYTPTDIDILQYALTLEHLENNFYSCALNNMDAQAFADAGFPAWVRNRFEQIAAHEASHVAVLSDALGADATKPCEYSFPYTDAKSFTALAQVIENVGVSAYLGAAGFIMDKTYLTVAGSILTTEARHQAWIASAVNKQNPWSGPYDTPLGLSDVYSIAAAFITSCPSSNPTLPVKAFPALTLSCDSAGSTATLNYTGADSSDTLILYSGLTTLALPITDMMVTIPSSLQGIAYAVVSSTSNTTMVDDSNTIAGPAIIDLPFASSASNPNFTGM

SEQ ID NO: 23 - DNA, 534 bp, Cryptococcus neoformans
  source 1..534: mol_type = genomic DNA
  CDS 1..534
atgtctctaactagtgtcactcgcgtcgtatccaaatccatcctcggcgcttcctttactagcacaactcgcaggcttaccactaccgttcccagatttggtagaatgcctcctcctgctcacaagatggcccacttcccgaggatcacatcctctcttccctcagaacactctgagtttagaacagtgatgtggacgggcgagagcagtcaacttgtcctcatgactatccctgtcggaggagaaataggggaagaaattcaccatgttgaccaacacttggttttcacctctggtactgccaaggccattgttggaggagaagaaaaagagatcaaggctggagatcttgtcatcgttcctcagggtaccaagcataacttcgtcaatacgggccctacccctctttgcctttttactgtatatgctccggccgagcatgccgagacaacagtcaacaaaacgaaggaggaaggggataaattggaagacgagggcaaggatgagcctccaaagtgggcagttaggaagtag

SEQ ID NO: 24 - protein, 177 aa, Cryptococcus neoformans
MSLTSVTRVVSKSILGASFTSTTRRLTTTVPRFGRMPPPAHKMAHFPRITSSLPSEHSEFRTVMWTGESSQLVLMTIPVGGEIGEEIHHVDQHLVFTSGTAKAIVGGEEKEIKAGDLVIVPQGTKHNFVNTGPTPLCLFTVYAPAEHAETTVNKTKEEGDKLEDEGKDEPPKWAVRK

SEQ ID NO: 25 - DNA, 1074 bp, Cryptococcus neoformans
  source 1..1074: mol_type = genomic DNA
  CDS 1..1074
atgccaactgtactcctcacaggtatcacgggatttctgtctgcgcacgtcgcccataccttcctgaagcatgactggatagtgcacggcacacttcggtccagctcgaaggtagcgttaatcgaagttattcctgaatactctccttatatttcgtcaggcaaactaaaactcttcgttgtcggacctcttgagaatgccgattacactgaagccatgaaaggcgttgatgctgtggtccacactgcgtctccggtagagtttggtggagacaattttagagagagccatttgaaacctgctttggaaggaacaaggggtgtcctcagagctgtagccaaagagaagaatgtaaagtccgtcgtctacactagtacttttggagccgttggtgatcataggtatcatcccactgagatcaaaggcaaagttatcactgaggataactggaacccgtataccttggaagagctggataagatggtggaatctggagagtcaggcaaccccacatttcctccaggatatctgttctataaaggagccaagaagtacgcggaactcgctgcttgggaatgccagaaagaagcgagagaacagggtgctgaatggtctttggccacgatgaactgtgtgatgatctgggggcctccaattcaacctctcacatcactcagtcatgggggcatgtcgaccgagttcctttggatgcttgcaggagggaaagatgcccatatcatggacagtctctatccctattacgtcgatgttcgggatgctgctgaagcacactatcaagccaccgtccgtagagcgcaaggaaggtttatcatctctgccggcccttatgatttccaagagttcgcagacatgcttagggagctttatcctgagcaaaaagaacgattcgcccttggtgctcccggcaaatatatgtacagagatccaggagtgtacgtgctcacaaatgaaaagagtcaaagggaacttggtattacttaccgtccaaaacaagagactctcaaagatgcatttgacaggtttttcgctttggagaaacaaggattgaagtaa

SEQ ID NO: 26 - protein, 357 aa, Cryptococcus neoformans
MPTVLLTGITGFLSAHVAHTFLKHDWIVHGTLRSSSKVALIEVIPEYSPYISSGKLKLFVVGPLENADYTEAMKGVDAVVHTASPVEFGGDNFRESHLKPALEGTRGVLRAVAKEKNVKSVVYTSTFGAVGDHRYHPTEIKGKVITEDNWNPYTLEELDKMVESGESGNPTFPPGYLFYKGAKKYAELAAWECQKEAREQGAEWSLATMNCVMIWGPPIQPLTSLSHGGMSTEFLWMLAGGKDAHIMDSLYPYYVDVRDAAEAHYQATVRRAQGRFIISAGPYDFQEFADMLRELYPEQKERFALGAPGKYMYRDPGVYVLTNEKSQRELGITYRPKQETLKDAFDRFFALEKQGLK

SEQ ID NO: 27 - DNA, 2781 bp, Cryptococcus neoformans
  source 1..2781: mol_type = genomic DNA
  CDS 1..2781
atgtttaggaagagaattctttatctttcttctttttcaatccctttgtacacagtcccagcccacagttattcctgtacttttcagaccaaccagcgcccttctactctcctcaaacgcgtacattcgctcgctatgtccttcccgccagtgcagcccgccgacaatggcatggcggtcgttgctcccaatctcgagtctaaccctaccactgttgcgtcccacgccccacaaattgccgtcaaggatgaaaatgatagtatgagcgaggatgagcagcctttggcgaaaagcaaagcgaatggagcgaggaagagagtcgaaaacagcagtgacgaggaagagaaacctctcagcaaaaagcccagagccaatggtgtcaacaagaaaagggtcgtcgccagcagtgatgaagaaagcgatgtttcacctcctgctaagaggcctgtttccaagcaatccaaacctgccacccccgattctgaatctgatgacgatcaacctctcgccaagaaggctaacggactggccgcatccaaacgtcaggctaaaaaagcggaggaattatcagaagaaagctcggaggaagaaaagcctcttgcgaaggttgccaagagggtatcagcaaagaagatgaagagcgagactgaggactctgaggaagaccggcctcttgcaaagaagaaggctcctgttaagcgtgctccagcaaagaaatcggcgaagaaggaacctagtgagagtgaagaggatgagaagcctttagcgaagaacgctagagggaaggccaaggcggcgacggtgaaggaagagaaaggtaagaagacaaagaaggagaaagaagaggaagaagaggaggaaaggtacaagtggtgggaacaggatgctttgggtgatgggtcatccaagtggacggtccttgagcacaacgctgttctcttccctcctccttatgttcctttacccaagaacgtgaaaatgaagtacgatggcgtctcacttaccctccctcccgagtctgaagaagtcgccggtttcttcggtgccctccttgaaaccgactatgctcaagatgccaaattccgtgaaaactttttccgagactttaaggctatcgtcgaaaaatatccacccaaggaggacgtcaaggttaagaagttggaaaagtgcgattttagaccgatgtttgagtactttgaaaaggagaaggagaagaagaaggcgttgactaaggaagagaaaaaggcgattaaagcggagaaggacaagcttgaagcaccgtatctctatgcgaatgttgatggaaggaaggaaaaggtcggcaacttccgtgcagaacctcctggattgttcaagggtcgtggtgaacatcccaagaagggtactgtcaagaaccgtctccgacctgaagatatcattatcaacattggcaaagaagctcctatccctgtgcccaacattcccggtcagtggaagggtatccagcatgataacacagtgacttggctcgctcattggaaggagaatgtcaacggtaacgccaaatacgtcttcttgagcgctggtagtgcgtggaaaggtcaaagtgatcgtgccaagtttgaaaaggcccgtgagcttatcaaacatgtcgacaaaattcgaaaagactacactgccgacctcaaatccaaagtcatggctgaccgacaacgtgccaccgccctgtactttatcgatcgtctggctctgcgagcgggtaatgaaaagggtgaagatgaagcggatactgtcggctgttgttctctgcgatacgaacacgtgacgctctctccaccgaatactatcatctttgatttcctcggtaaggactcgatgaggttccatcaggaagtcgaggtcgatccgcaagtgttcaagaacataaaactgtttaaggctgatccgaagaagaagggtgacgatatctttgaccgactgaccaccactcttcttaacaagcacctcaacagcatgatgcctggtcttaccgccaaggttttccgtacctacaacgcctcatggactttccaagaacaactcaaaaacacacctaagaacggaactgtagccgagaagattgcggcgtacaacactgccaatagggatgttgccatcttgtgtaatcaccaaaagagtgtcagcaagggttttgagggcagttttgccaaagccgaggataagattcgtgccctcaagtatcagcgtctcaagcttcgtctccaacttttttctcttaaccccaagattaagaagaagcatcccgagcttgcggaggatgagtctgatgtggatgacgaatttatggagcgccacgaagccgaattactcgaaaaagctttggagaacgcaaagaagaaatgggatacggataatgtcaagcttgaaggggatgggaagaaaaagaagacgaagggagagttggatgagaggttgagtgagatcaaggcagagtttaaggagttgaagaaggagaggaaggctaaaaagattgatgccaagagaggagccacggaggagaaacttcttgctcaggtcgccaggatcgacgaacgtatcgctaccgccaaagtccagcttcaagatcgagacaagctcaaggatgttgctttgggcacatccaagattaactatatcgatccaagactaactgtcgcgtgggcgaagaagtttgatgttcctctcgaaaaactgttctccaaaaccctgcgagaaaagttcccttgggctgaggcggaggctggaccggactgggttttctag

SEQ ID NO: 28 - protein, 926 aa, Cryptococcus neoformans
MFRKRILYLSSFSIPLYTVPAHSYSCTFQTNQRPSTLLKRVHSLAMSFPPVQPADNGMAVVAPNLESNPTTVASHAPQIAVKDENDSMSEDEQPLAKSKANGARKRVENSSDEEEKPLSKKPRANGVNKKRVVASSDEESDVSPPAKRPVSKQSKPATPDSESDDDQPLAKKANGLAASKRQAKKAEELSEESSEEEKPLAKVAKRVSAKKMKSETEDSEEDRPLAKKKAPVKRAPAKKSAKKEPSESEEDEKPLAKNARGKAKAATVKEEKGKKTKKEKEEEEEEERYKWWEQDALGDGSSKWTVLEHNAVLFPPPYVPLPKNVKMKYDGVSLTLPPESEEVAGFFGALLETDYAQDAKFRENFFRDFKAIVEKYPPKEDVKVKKLEKCDFRPMFEYFEKEKEKKKALTKEEKKAIKAEKDKLEAPYLYANVDGRKEKVGNFRAEPPGLFKGRGEHPKKGTVKNRLRPEDIIINIGKEAPIPVPNIPGQWKGIQHDNTVTWLAHWKENVNGNAKYVFLSAGSAWKGQSDRAKFEKARELIKHVDKIRKDYTADLKSKVMADRQRATALYFIDRLALRAGNEKGEDEADTVGCCSLRYEHVTLSPPNTIIFDFLGKDSMRFHQEVEVDPQVFKNIKLFKADPKKKGDDIFDRLTTTLLNKHLNSMMPGLTAKVFRTYNASWTFQEQLKNTPKNGTVAEKIAAYNTANRDVAILCNHQKSVSKGFEGSFAKAEDKIRALKYQRLKLRLQLFSLNPKIKKKHPELAEDESDVDDEFMERHEAELLEKALENAKKKWDTDNVKLEGDGKKKKTKGELDERLSEIKAEFKELKKERKAKKIDAKRGATEEKLLAQVARIDERIATAKVQLQDRDKLKDVALGTSKINYIDPRLTVAWAKKFDVPLEKLFSKTLREKFPWAEAEAGPDWVF

SEQ ID NO: 29 - DNA, 1125 bp, Cryptococcus neoformans
  source 1..1125: mol_type = genomic DNA
  CDS 1..1125
atggcccaccaccacttcgtaggcataaacccggcaggcctctccttctcccatcccaccccgccagcagaccaccccgcgcccccctcctcgggcagcatccacaccccagcaaacttcgccagcattcaagaacccatcacagacccatccgctgtcgccgcccgccgacgcggtcgtccttccacaaggggcgaagctggcgtcactccgcccccagagatcggatggtgggaggaccgtgcgcccagctggcacaaggatgccatgcagggcggcaagtcttctatggagctcctgatggaatggtcagaggagatgaagaatcaaggccactactactggatgggcgtcagggatggcggcaatctgcatcaaggtgcttcgcgttttagggactacttatatgcccagcatggtcctattaggcggtcaagtaaggctatcagaaataaagtggagaatattaagcaaaagttctttgaagcccaggaatggctcaaggatcccaatggggaccataccaccatgaccattccggacgtcgaaaaaaaactcaacaagatctgtcgcaactaccgcttctgggaaaccatcttcgtagagcttcctccagttgaccacgaagctggccagaatgccgaagggtcttcatccaaccagactcttcagtctgcttctcaaactgccgttcgacagggtaatgggcccctcatccgcggtattcctgttccggaaatgggacaggcagcggccgatgatgcgcagcgaaatgtccgccgacgccttaatgatggcagctctgccactattccttccgactcatctttggttggtcgcgtgctccctgctagctacctcgaacgcacccgtgaagaacgcgatcgcgaaaagcatgaattagcgaaaaagcagcaagccctcaacagggaacaatatgaactcgagcagaagaaagatgagagagatcagaagagatttgagtgggagcagactaagcatctggtggagacggctttaaaaatccgagaattggatatcattccgttggaagcggcgatgatcaaagctagagctctttatggccaggcgcgagaggaagatcaagctgaagctactctttaa

SEQ ID NO: 30 - protein, 374 aa, Cryptococcus neoformans
MAHHHFVGINPAGLSFSHPTPPADHPAPPSSGSIHTPANFASIQEPITDPSAVAARRRGRPSTRGEAGVTPPPEIGWWEDRAPSWHKDAMQGGKSSMELLMEWSEEMKNQGHYYWMGVRDGGNLHQGASRFRDYLYAQHGPIRRSSKAIRNKVENIKQKFFEAQEWLKDPNGDHTTMTIPDVEKKLNKICRNYRFWETIFVELPPVDHEAGQNAEGSSSNQTLQSASQTAVRQGNGPLIRGIPVPEMGQAAADDAQRNVRRRLNDGSSATIPSDSSLVGRVLPASYLERTREERDREKHELAKKQQALNREQYELEQKKDERDQKRFEWEQTKHLVETALKIRELDIIPLEAAMIKARALYGQAREEDQAEATL

SEQ ID NO: 31 - DNA, 1251 bp, Cryptococcus neoformans
  source 1..1251: mol_type = genomic DNA
  CDS 1..1251
atggacgccaccactcttacagggcacgtcaaggagctcaatgctgcaaaccaagcgggaaaatcagatgaagttatctctctgctcaagaaacttcaggctgaggttgttcctacagaagatctccttcgatcatcgaaagctggtgtcgcagtcggcaagcttcgtacccacgccacaccatcagtctcaagtcttgccaaggagatagttaagaagtggagagatgcggtcgaggagacaaagaagaagagaaaaagagcagaaggtgatgaaggaaaagatgtaaagaaggagaaggaggaagggaacgggaaacgagtcaaggcggaaacggggtcattagcggcgacaccatcagctagcacacccgcctcggcctctacacccgatgtcaaagcgacctcccctcctgtccgtcaacctctttcaaccattgactcatcacgcactacgcctcgaaccgccaaaagcgatggagtggccgacagcctgagagctgattcgagcgaaggaggcagtgtagatagcgtgagggacaagtgtgtgatcatgatttatgacgcattggcgttggatagcacggctgaaataaagattttgaaagagcgcgccattggaattgagcgcgcagcgaataaagctatgaacttctcaacaggaaacgattatcgcgctaaaatgagatcactattcctcaacttgaaagacaagggtaatcccgctttgagaaacgagattgtcttgggctacgtcagcaccgaaaaagtcgctagcatgtccaaagatgaaatggcctctgaaagcgttcgaatgctaaaggagaagattgcgagtgacaacttgttcaaggccaaggctgtcggagtcacccaagctgagacagacgcgttcaagtgcggacggtgtcaccagaggaaatgtacttattaccagatgcagacaagaagcgcggatgaacctatgactacttttgttacgtatgtgtctgacctgactccaaaagaatcattgctgactacgtgtacgacttgctctttttattcagatgtactaattgtaacaacaggtggaaattcagctagtttcggattttgcctctggggagcattgtatctttcgggttttttgtcacgtcgtctatgcagccagtatatttacgaggcgtatcgttgtgatttgcgtgtcaatgtcacaaagccattaagtgccgtaaatatgcctttttgcagtgttctgaaattcaaatga

SEQ ID NO: 32 - protein, 416 aa, Cryptococcus neoformans
MDATTLTGHVKELNAANQAGKSDEVISLLKKLQAEVVPTEDLLRSSKAGVAVGKLRTHATPSVSSLAKEIVKKWRDAVEETKKKRKRAEGDEGKDVKKEKEEGNGKRVKAETGSLAATPSASTPASASTPDVKATSPPVRQPLSTIDSSRTTPRTAKSDGVADSLRADSSEGGSVDSVRDKCVIMIYDALALDSTAEIKILKERAIGIERAANKAMNFSTGNDYRAKMRSLFLNLKDKGNPALRNEIVLGYVSTEKVASMSKDEMASESVRMLKEKIASDNLFKAKAVGVTQAETDAFKCGRCHQRKCTYYQMQTRSADEPMTTFVTYVSDLTPKESLLTTCTTCSFYSDVLIVTTGGNSASFGFCLWGALYLSGFLSRRLCSQYIYEAYRCDLRVNVTKPLSAVNMPFCSVLKFK

SEQ ID NO: 33 - DNA, 657 bp, Cryptococcus neoformans
  source 1..657: mol_type = genomic DNA
  CDS 1..657
atggattaccaaaatcgagcaggtgcaaacaagggtagtggtggtgtcgctggtgcatccgagacagcagtggacaggagagaacgtcttcgaaaacttgctttggagactattgacttggccaaagatccctatatccttaggacccatctcggtacattagaatgccgtctttgtctcactcttcacgtcaacgagggttcttaccttgcccacactcaaggaaagaaacatcaaacaaaccttgctaggcgtgcagccaaggacaacaaggatcagacattaatgatccaagctcccacagccgcgcaacaagtgaagaagaaagtgtttgttaagattggaagacctggatacaaaatcatcaaaattcgagagcctgtcagtcaaaggatgggtttattattcactgtgtctttacctgagataaaagcgggagagaggccaagaaggaggttcatgtctgcttttgaacaacggcgagagattcccaataaagctttccagtacttagttttggcagccgagccatacgagaccatagcatttgccatcccctcaaaagagatggttgacgttgatgaagacccggagtcgacatgggagcactgggatgccgacgagaaggtttacagttgtcaattcttgtataaataa

SEQ ID NO: 34 - protein, 218 aa, Cryptococcus neoformans
MDYQNRAGANKGSGGVAGASETAVDRRERLRKLALETIDLAKDPYILRTHLGTLECRLCLTLHVNEGSYLAHTQGKKHQTNLARRAAKDNKDQTLMIQAPTAAQQVKKKVFVKIGRPGYKIIKIREPVSQRMGLLFTVSLPEIKAGERPRRRFMSAFEQRREIPNKAFQYLVLAAEPYETIAFAIPSKEMVDVDEDPESTWEHWDADEKVYSCQFLYK

SEQ ID NO: 35 - DNA, 813 bp, Cryptococcus neoformans
  source 1..813: mol_type = genomic DNA
  CDS 1..813
atgacagtcaaggcagagcaagatctctatcttgacccctccattcgggattgggtccttatccctatcaccctaatcatgctactcgtcggtgtgttaagacactacatcacgcaattccttaactctgcaccaaaaaaacaaacagcagctgccgttcgcgaacaacgcgcacttggtcgctcagctctgcttcgggcaactgcgactctgtccccccttccgcctgcctcttacaaggctctctcgggatcccttgctgcttcactttctactggtgagtatatcaagcccgccccagagtcaaagggggatgcttctcccgccaatcctctcgaaggtgctgggatggaaaatgcgatggacggtatgaaaaagcaggccgtaatgatggtacccaacatggttatcatgcagtatatcaacgtctttttttccggatttatccttatgcgtctgccatttcctttaaccgcaggctttaagtcgttgctgtcaagggatattcccatggctgatctcgatgtgcgatgggtttccgctttgtcctggtattttctcaacttgtttggcttgaacggtgttttcaaactaattcttggagctgagaatgctgctgtagacagccgtgacctcacctcgctgtctgcactttctggggcaggaggccctatgcccggccccggcggtccaccagacatggtcaagcttttcaaggccgaggttgagaacttggcattggcagaaagttcatacaagtgggtcggcgacggagtagaagatagagttttgagagcttggggcaaagtttaa

SEQ ID NO: 36 - protein, 270 aa, Cryptococcus neoformans
MTVKAEQDLYLDPSIRDWVLIPITLIMLLVGVLRHYITQFLNSAPKKQTAAAVREQRALGRSALLRATATLSPLPPASYKALSGSLAASLSTGEYIKPAPESKGDASPANPLEGAGMENAMDGMKKQAVMMVPNMVIMQYINVFFSGFILMRLPFPLTAGFKSLLSRDIPMADLDVRWVSALSWYFLNLFGLNGVFKLILGAENAAVDSRDLTSLSALSGAGGPMPGPGGPPDMVKLFKAEVENLALAESSYKWVGDGVEDRVLRAWGKV

SEQ ID NO: 37 - DNA, 1365 bp, Cryptococcus neoformans
  source 1..1365: mol_type = genomic DNA
  CDS 1..1365
atgaccgcggtgaacagcaaccagggcaccggcaaactgagcggccgcgtgggcattgtgggcaccggccatcgcgcgcgcctgtataccaccgcggtggcgagccgcgcgaacaccagcctggtggcgctgtgcgataccaacgatgcgcgcatggattggcataacaaaatgctgcgcgaagcgggccgcccggaagcgaaaaaatatgcggcggaagattttcgcaaaatgctggaacaggaaaaactggatgtgctggtggtgaccaccattgattatacccatgatatgtatattattccggcgctgaaagcgggcattaaagtgctgagcgaaaaaccgatgaccaccaacgtggataaatgcaaagcgattctgaacgcggtgaacgaaagcaaaggcagcctgaccgtgctgtttaactatcgctataacccgattcattggaaagtggcggaagtgattgcgaaaggcgaaattggcgaagtgaaaagcgtgcattttgaatggctgctggataccgtgcatggcgcggattattttcgccgctggcatcgctataaagatcgcagcggcggcctgatgattcataaaagcagccatcattttgatctggtgaacttttggattcagagcgtgccgcagagcgtgtttggcatgggcagcctggcgttttatggcaaagaaaacggcaaaaaaagcggctggggcaaaaactatgaacgcgcgcgcgatgcgaaagaagcggaaaacgatccgtttgcgattcatctgggcgatgaagaaggcctgaaaggcctgtattttgatgcggaacatattgatggctatcatcgcgatatgaacgtgtttgcggatgatattaccattgaagatgatatgagcgtgctggtgcattatgaaagcggcgtgaacatgacctatcatctgaccgcgtatagcccgtgggaaggctatcgcgtgatgtttaacggcacccatggccgcctggaactggaagtggtggaaaacgcgtttcgcctgccgattccgaaaggcagcaacaacgcgagcgaacatgtgcatggcgatagcgcgctgccgaacgaaggccatagcaaaattaccctgcataaactgtggcagcagccggtgaacgtgccgtatcaggaagcgaaaggcggccatggcggcggcgatgaagcgatgctggatgaaatttttggcccgaaagaaggcgaagaagaacgcaaatgcccggtgaacggcctgagcgcggatcagaaagatggcgcgctggcgatggcggtgggcctggcggcgaacgaaagctttaaaaacggcaaacaggtgtttattaaagaactgctgggcggcaccctg

SEQ ID NO: 38 - protein, 455 aa, Cryptococcus neoformans
MTAVNSNQGTGKLSGRVGIVGTGHRARLYTTAVASRANTSLVALCDTNDARMDWHNKMLREAGRPEAKKYAAEDFRKMLEQEKLDVLVVTTIDYTHDMYIIPALKAGIKVLSEKPMTTNVDKCKAILNAVNESKGSLTVLFNYRYNPIHWKVAEVIAKGEIGEVKSVHFEWLLDTVHGADYFRRWHRYKDRSGGLMIHKSSHHFDLVNFWIQSVPQSVFGMGSLAFYGKENGKKSGWGKNYERARDAKEAENDPFAIHLGDEEGLKGLYFDAEHIDGYHRDMNVFADDITIEDDMSVLVHYESGVNMTYHLTAYSPWEGYRVMFNGTHGRLELEVVENAFRLPIPKGSNNASEHVHGDSALPNEGHSKITLHKLWQQPVNVPYQEAKGGHGGGDEAMLDEIFGPKEGEEERKCPVNGLSADQKDGALAMAVGLAANESFKNGKQVFIKELLGGTL

SEQ ID NO: 39 - DNA, 1338 bp, Cryptococcus neoformans
  source 1..1338: mol_type = genomic DNA
  CDS 1..1338
atgagcgcgattcgcgcgctgaacatgcgcaaaaccgcgagcgcgctgaaagcgccggtggcgtttaaacgcaccctggcgaccccggtgaacagcctgtataccagcgtgctgccggcgaaaattccggcggcgctgcatctgaaaagcggccagagctattttggcagcagctttggcagcgaaaacagcaaatttggcgaaaccgtgtttagcaccagcattaccagctataccgatagcatgaccgatccgagctatctgggccagattctggtgtttaccagcccgatgattggcaactatggcgtgccgagcaacaccagcagccagtttccgggcattccgtttctggaaagcgaaaaaattcagtgcaccggcgtggtggtgagcgatgtggcgctgaaatatagccattatcaggcggtggaaagcctgcatgaatggtgcaaacgctatgatgtgccgggcattaccggcgtggatacccgcgcgattaccagcctgctgcgcgatcagggcaccaccctgggccgcctggcggtgggcgatgaagcgggcaaaccggcgccgcaggaagcggaatattgggatccgagcaaagaaaacctggtggcgcaggcgagcaccaaaaaagcgtatgtgctgaacgaaaaaggcagcggcccgcgcattgcggtgctggattttggcaccaaagcgaacattctgcgcagcctgattcgccgcgatgcggtggtgaccgtgctgccgtgggattttgattttaacaccgtgcgcgatcagtttgatggcctgtttctgagcaacggcccgggcgatccgaaaatgattatggatagcgcgatgcgcgtgcgccagaccattaacgaatggaacaaaccgatttttggcatttgcatgggccatcaggtgctgggcctggcggcgggcctggaagcgtatcgcatgacctttggcaaccgcggccataaccagccggtgctggcgctggcgagcagcggcagcattaaagcgggccgcgtgtatgtgaccagccagaaccatcagtatgcgctgcgcctgaccgaagattttccggaaggctgggcgccgttttttattaactgcaacgatagcagcgtggaaggcattattagcaccccggaaagcggcaaacgcatttggggcgtgcagtttcatccggaaagcgcgggcggcccgctggataccattgaaatgtttaccgattttgtgaacgaatgcgatgtgagccgcaaaggctttagcggcagcgcgatgattgcgaacgaagtgaaagtggatggccatgcggcgaaagcggcgagcgtgagcgcg

SEQ ID NO: 40 - protein, 446 aa, Cryptococcus neoformans
MSAIRALNMRKTASALKAPVAFKRTLATPVNSLYTSVLPAKIPAALHLKSGQSYFGSSFGSENSKFGETVFSTSITSYTDSMTDPSYLGQILVFTSPMIGNYGVPSNTSSQFPGIPFLESEKIQCTGVVVSDVALKYSHYQAVESLHEWCKRYDVPGITGVDTRAITSLLRDQGTTLGRLAVGDEAGKPAPQEAEYWDPSKENLVAQASTKKAYVLNEKGSGPRIAVLDFGTKANILRSLIRRDAVVTVLPWDFDFNTVRDQFDGLFLSNGPGDPKMIMDSAMRVRQTINEWNKPIFGICMGHQVLGLAAGLEAYRMTFGNRGHNQPVLALASSGSIKAGRVYVTSQNHQYALRLTEDFPEGWAPFFINCNDSSVEGIISTPESGKRIWGVQFHPESAGGPLDTIEMFTDFVNECDVSRKGFSGSAMIANEVKVDGHAAKAASVSA

SEQ ID NO: 41 - DNA, 843 bp, Cryptococcus neoformans
  source 1..843: mol_type = genomic DNA
  CDS 1..843
atgagcgtgccgagcctgaaaagcgcgctgaaaaaaccgaccaaaagctttgataccccgccggcgggcccgagcaaactgagcgtggcggcggcggtgccggaaaaaagcaaagcgaaagcgaaacagagcgtgagcattgcggaaaaaccgcagcgcctgcgcggcccggatctggaaagcgaaagcgaaggcaacgcgagcggctttgaagatgaaagcgcgagcgaagtggaagtggatgaagatgaagaaatgaacaccgatgaagaaattgaaaaagcgaaagaaggcaaaccgaaaaaaagcaccaaacgcaaaaaagcgccgaccaccgcggcggattttggcgcgaccctgaccagcctgctggcggatccgctgaccaaaagcaacaaaaaagcgaaaaccgcggatagcaccaaaaaagcggcggcggcgccgattctggcgctgagcgcgcataaactgccgaccaaagcgagcgtgagcctggaagcgaaagcgaaacgccagctgaaagcggaaaaagaagaaaaagaagatcgcgcgcgcgtgcagaacgtgctggaaggctggagcggcgatggcgtggtgggcggccaggaatttgaacgcaacctgcgcaaaaccgcgcagaaaggcgtggtgaaactgtttaacgcgattctggtggcgagcaaaaacgcggaagcggcgcagaccaccctgagcgaaaaagcgcgcctgaaaccggaagcggcgaaaaaaaaagaaaaagataacattctgggccgcggcggcaaagaagatgtgctgaccaaagaaagctttctggaaatggtgcgcaaaggcagcagcaaa

SEQ ID NO: 42 - protein, 281 aa, Cryptococcus neoformans
MSVPSLKSALKKPTKSFDTPPAGPSKLSVAAAVPEKSKAKAKQSVSIAEKPQRLRGPDLESESEGNASGFEDESASEVEVDEDEEMNTDEEIEKAKEGKPKKSTKRKKAPTTAADFGATLTSLLADPLTKSNKKAKTADSTKKAAAAPILALSAHKLPTKASVSLEAKAKRQLKAEKEEKEDRARVQNVLEGWSGDGVVGGQEFERNLRKTAQKGVVKLFNAILVASKNAEAAQTTLSEKARLKPEAAKKKEKDNILGRGGKEDVLTKESFLEMVRKGSSK

SEQ ID NO: 43 - DNA, 600 bp, Cryptococcus neoformans
  source 1..600: mol_type = genomic DNA
  CDS 1..600
atgacctgcgcgctgattccgctgctgcgcaaaagcgatctgcgcagcgtggtgattattgcgagcattgcgggcctggcgaaccagcgcgcgaccggcagcgtgagctatggcgtgagcaaagcggcggcgattcatctgggcaaactgctggcgggccgcctgcatccgctgaaaattcgcgtgaacaccatttgcccgggcatttttccgagcgaaatgaccggcaaaaacgatgcgggccagggcctggaatatgatattggcgaaattccgaccaaagcggcgaaacgcagcaccgtgggccgcccgggcctgccggaagaaattgtgggcccggtgctgctgctgagcagcaaagcgggcggctattttgatggcgcgatgctgaccgtggatggcggccgcctgatggtgagcggcccgttttgctttgtgtttagcccgagcagcctgccggtggataacctgcgctgctttctgctgctgaccaccgtgggcaaccgcgtgccggcgtttatgatggtgagcgattgcctgcgcattcgcatttttgaactgaacgaacagcgcaaaaacgaaaccgatgtggaaatgggc

SEQ ID NO: 44 - protein, 200 aa, Cryptococcus neoformans
MTCALIPLLRKSDLRSVVIIASIAGLANQRATGSVSYGVSKAAAIHLGKLLAGRLHPLKIRVNTICPGIFPSEMTGKNDAGQGLEYDIGEIPTKAAKRSTVGRPGLPEEIVGPVLLLSSKAGGYFDGAMLTVDGGRLMVSGPFCFVFSPSSLPVDNLRCFLLLTTVGNRVPAFMMVSDCLRIRIFELNEQRKNETDVEMG

SEQ ID NO: 45 - DNA, 1440 bp, Cryptococcus neoformans
  source 1..1440: mol_type = genomic DNA
  CDS 1..1440
atgctggtgaacagcagcagcatgctggtgacccgcacccatggcctgatgggcgatgaagcgtggaaagaactggcgaaatatggcctgacccgctggagcgatgatggcgcgtttctgaccgtgccggcgcgcggctgcagcatgtgcgaagtggatccggtgctgtgcgaagaaattggcgaagataacctgaaacgcagcctggcgtttagcggcaccaaccgccgcctgaaacgcgtgctggcgaaactgcgccgcggcgaaaccattaacgtgggcgcgattggcggcagcgtgaccaaaggctatggcctgaaccgctataacgaaccgtattatccggataccccgaccaacctgcatcgcattatttttgatcatctggtgagcctgtatccggcgccgaacggcgtgaaaaccgatgatagcggccgcaaagaaggcaaacatggctatattaacggcggccagggcgcgaccggcaccggctattttagctattgctgggaagaacatgtgccggcggatctggatctgatttttctggaacaggcgattaacgatgaactgctgctgcgcaacattgatagctatgaactgctggtgcgcagcctgctggatctgccgaccagcccggcgattgtgaacctgcatgtgtttgcgctgatgtttaacagcattaccctgggcggcgatctgcatcagagcattgcgcagttttatgatctgccggtgctgagcctgcgcaacgcgctgctgaacgatatgctgaaaaacgaaagcctgattagcgaatatttttttgtgcatccggaaggcgatattgatctgcgccatattagccgcaaaggccataacgtgatgggccgcattggcgcggcgtatatggatagccagatttgcgaaatggataaatatgaacagggcattccgggcgcggatagcatgagcattgatcagctgtatccggtggaaccgattccgcgcatgcagattaacatgaaatatgataaagatctggtgctgccgaccattaaaccgcagtgctttagcgcgaacagcgaaaaacatccgctggtgccggtggaaaacaacggctggcgcaaatggaactggaaagaaaaacattatctggtggcggatgtgccgggcagccgcgtgagctttaaactgaaaaccaacatgggcaaaattgaagtgcagtatctgcgcagctatcagtatcatcagggcagcgcgaaatgctgggtggatgaagaagtggaaaaagcgattaaactggatggctattggaaagaaccgtataacattggccgcgcggtgaccattcgcgaaggcctggaaccgggcgaacataccctgacctgcgaactgctgaaacagaccgcggatccggaaggcggcctggaatttcgcctgattagcattatgagcatt

SEQ ID NO: 46 - protein, 480 aa, Cryptococcus neoformans
MLVNSSSMLVTRTHGLMGDEAWKELAKYGLTRWSDDGAFLTVPARGCSMCEVDPVLCEEIGEDNLKRSLAFSGTNRRLKRVLAKLRRGETINVGAIGGSVTKGYGLNRYNEPYYPDTPTNLHRIIFDHLVSLYPAPNGVKTDDSGRKEGKHGYINGGQGATGTGYFSYCWEEHVPADLDLIFLEQAINDELLLRNIDSYELLVRSLLDLPTSPAIVNLHVFALMFNSITLGGDLHQSIAQFYDLPVLSLRNALLNDMLKNESLISEYFFVHPEGDIDLRHISRKGHNVMGRIGAAYMDSQICEMDKYEQGIPGADSMSIDQLYPVEPIPRMQINMKYDKDLVLPTIKPQCFSANSEKHPLVPVENNGWRKWNWKEKHYLVADVPGSRVSFKLKTNMGKIEVQYLRSYQYHQGSAKCWVDEEVEKAIKLDGYWKEPYNIGRAVTIREGLEPGEHTLTCELLKQTADPEGGLEFRLISIMSI

SEQ ID NO: 47 - DNA, 1176 bp, Cryptococcus neoformans
  source 1..1176: mol_type = genomic DNA
  CDS 1..1176
atgagctttgatgcggtggtgattggcagcggcgtgattggcctgagcattgcgcgcgaactgcataaccgcggcctgaaagtggcgattgtggcgcgcgatctggcggaagatagcattagcgtgggctttgcgagcccgtgggcgggctgcaactggtttagctttgcggaaggcggcaccccggcggcggaatgggataccattacctttggcaaactggcgaaactggcgaaagatcatccgcatatttgccagaaaattccgttttgcagcgtgtgggatctgccgaaaagcgatgcggaaagcgaaccgtggtttaaagatctggtgtttgattataaaaacctgaaaagcaccccgggccagccgctgccgggcggcaaaaaatttggccatagctttgcgagctatgtgctgcatgcgccgaactatattcgccatctgagcagcgaaacccgcgcgctgggcattccggtgcatcgctatcgcctgagcagcctggatgaagcgtataacctgagcggcattggcaaagtgagcctggtggtgaacgcgagcggcctgggcgcgaaagcgctgattggcgtggaagatgaaaaagtgtatccgggccgcggccagaccgtgctggtgcgcgcgccgggctttaaagcgtgcattatgcataccgaaggcttttatgcggatctggatgaaagcggccgcgaagtgaccccgccgccgccggcgtatattattccgcgcccgggcccggaaggccatgtggtgctgggcggcgtgtatcagcgcgataactggagcaccctgccggatctgaaagaagcggaacgcattctgaaagattgctataacctggcgccggaactggcgggcccgaacggcaaaacctggaaagatattgaaattattagccataacgtgggcctgcgcccggcgcgcgaaggcggcccgcgcctggaaattgaagaacgcgaagtgggcaccggcgcgaacgaaggcaacgcgtatgatgtggcgccgatgattggccgcattggcgaacgccgcaaagtggcggtggtgcatgcgtatggcattggcagcgcgggctttcaggcgagcctgggcatggcggaaaaagcgagcgatctgaccgtgaaatatctgagcggcaaacgcagcccggcgcgcctg

SEQ ID NO: 48 - protein, 392 aa, Cryptococcus neoformans
MSFDAVVIGSGVIGLSIARELHNRGLKVAIVARDLAEDSISVGFASPWAGCNWFSFAEGGTPAAEWDTITFGKLAKLAKDHPHICQKIPFCSVWDLPKSDAESEPWFKDLVFDYKNLKSTPGQPLPGGKKFGHSFASYVLHAPNYIRHLSSETRALGIPVHRYRLSSLDEAYNLSGIGKVSLVVNASGLGAKALIGVEDEKVYPGRGQTVLVRAPGFKACIMHTEGFYADLDESGREVTPPPPAYIIPRPGPEGHVVLGGVYQRDNWSTLPDLKEAERILKDCYNLAPELAGPNGKTWKDIEIISHNVGLRPAREGGPRLEIEEREVGTGANEGNAYDVAPMIGRIGERRKVAVVHAYGIGSAGFQASLGMAEKASDLTVKYLSGKRSPARL

SEQ ID NO: 49 - DNA, 198 bp, Cryptococcus neoformans
  source 1..198: mol_type = genomic DNA
  CDS 1..198
atgccgggctttcaggatgtgatttataacaccttttttcgccgcaacagcgtgtttgtggcgaccacctttattgcggcgtttagctttagcatgggctttgatctggcgaccaccgcgttttgggatagccataaccgcggcaaacagtggaaagatattcgccataaatatattgaagcggcgggcgatgatgaa

SEQ ID NO: 50 - protein, 66 aa, Cryptococcus neoformans
MPGFQDVIYNTFFRRNSVFVATTFIAAFSFSMGFDLATTAFWDSHNRGKQWKDIRHKYIEAAGDDE

SEQ ID NO: 51 - DNA, 1368 bp, Cryptococcus neoformans
  source 1..1368: mol_type = genomic DNA
  CDS 1..1368
atgagcagcggcaacgtgccgaaaattgaacgcctgagctgcgtgccgaacgattatccgtggggcgaagtgggcaacgatagcctggcggcgcgcctggcgagcaaaaacggcgcggtgagctttgatctgaaaccggaacaggcgtatgcggaactgtggatgggcacccatccgaacaacccggcgcatctgtttagcagcccggataccctgctgagcacccatctgaaaaaaaacccgagcctgctgggcgcggcgaaccgctttagcccgccgtttaccggcgcgaaaggcagcggcgcggaaggccaggaagaaggccatgtgccgtttctgtttaaagtgctgacctgcaaacaggcgctgccgctgcagattcatccggataaagcgctggcgaaaaaactgcatgaagaaaacccgaaacagtttggcgatattaaccataaaccggaaattgcggtgtgcctgagcgatcgctttctgggctttgcgagctttcgcccgtatgataaaattgcgagcctgctgaaaagcgtgcaggaaattagcctgctgccgagcctgctgcagaaaagcattaaaagctttattagcgcgccgagcgcggaaaccctgcagccgacctgggaaggctttattaaactgggcgataacgaagaaagcgtgaaaaaatttagcgatcgcgtgctgagccagggcctgaaagcgtttgatagcgtggatattgaagatgaagataaaaaccgcctggtgcgcgcggtggaactgggcaaaaaatataacccgggcgatgcgggcctgtttagcagcctgctgtttctgaacctgattgaactgaaaaaagatcagggcatgtatgtgggcgcggatggcccgcatgcgtggctggaaggcgaaattgtggaactgatggcgattagcgataacgtgctgaacgtgggctttaccagcgatgatagcaaagatgatccgagcctggtggcgaaagcggtgacctgcaccccgaaagcgattaaagatctgctgctggatgcgagcaaatatagcaaaagccagaacggccgcaccaccgtgtatagcaccccgtttgaagaatttagcattatgaaaattgcgggcgatgaaattctgagcccgctggatggcgcgggcgtggcggtggtgctggaaggcgaatggaccgtggaagatcaggaaggcaccaaacgcggcggcgaaggcaccgatggcgaaggcggcgaaggcaccatttggtttattggcagcgcgaccgaaaccaaatggaccgcgaaaggcggcaaaggccagatttggattgcgttttatgataaaaccgcgaaaaaagatgatgtgggcaaaaaa

SEQ ID NO: 52 - protein, 456 aa, Cryptococcus neoformans
MSSGNVPKIERLSCVPNDYPWGEVGNDSLAARLASKNGAVSFDLKPEQAYAELWMGTHPNNPAHLFSSPDTLLSTHLKKNPSLLGAANRFSPPFTGAKGSGAEGQEEGHVPFLFKVLTCKQALPLQIHPDKALAKKLHEENPKQFGDINHKPEIAVCLSDRFLGFASFRPYDKIASLLKSVQEISLLPSLLQKSIKSFISAPSAETLQPTWEGFIKLGDNEESVKKFSDRVLSQGLKAFDSVDIEDEDKNRLVRAVELGKKYNPGDAGLFSSLLFLNLIELKKDQGMYVGADGPHAWLEGEIVELMAISDNVLNVGFTSDDSKDDPSLVAKAVTCTPKAIKDLLLDASKYSKSQNGRTTVYSTPFEEFSIMKIAGDEILSPLDGAGVAVVLEGEWTVEDQEGTKRGGEGTDGEGGEGTIWFIGSATETKWTAKGGKGQIWIAFYDKTAKKDDVGKK

SEQ ID NO: 53 - DNA, 1962 bp, Cryptococcus neoformans
  source 1..1962: mol_type = genomic DNA
  CDS 1..1962
atgacccgcgattttgatagcctgttttttaccggcccgatttttgtggattatagctttaccgcgcgccatctggaaagctttctgagcagctttccgccgcaggtgagccatgcgatgagcagcgcgagcaccccgaccgcgccgtatctggaagatctggtgcgcaacagcctggatcagaccctgccgtgggtggtgcagaaatatggcggcaccagcgtgggcaaaagcctggataacattaccaaaattgtgggcagctatattgataacggcagcaaagtggcgattgtgtgcagcgcgcgcagcacccagaccaaaagcctgggcaccaccaacctgctgctgcaggcgagccgcgaagcgctgcagccggcgctgagcagcagcggcgatggccgcagcggcagcatgagcggcaccgcgaccccgttttatccgaaacgcgtgggcagcggcttttttggcaaagatcagagcaccagcatggtgagcagcgtgagcagcctgagccagctggaaccgcagctgggccgcagcggcagcccgagcccgtttcagagcagcagcagccgcagcccgccgcgcagcccggcgaccccgagccaggatagcagcgtgagccaggaaccggcgtttcatgcgaccgtggatctgattaaaaaaggccatctggaagcggcgcgcgcgagcctgaaagaaggcccgctgcgcgatgaactggaagaagaaattgaacgcgattgcgaaagcctgcgcagctttctgtatgcggcgcagattattgatgaaattagcccgcgcagccaggatagcattgtgggcaccggcgaacgcctggcgtgcaaaattgtggcggcggcgctgcgcgatcgcggcgtggatagcgaactggtggtgctggataacattgtggatgcgagcatgagcgcggcgagcgaagcgattagcgtggatgcgggcgatcagggcgtggcgcagctgggccaggaattttatgatcagctgagctttcgcctgggcgaacgcctgcgcgaatgcggccagcgcgtgccggtggtgaccggctattttggcccggtgccgggcagcctgctggcgcagattggccgcggctataccgatctgtgcgcggcgctgtgcgcggtgggcctgaaagcgagcgaactgcaggtgtggaaagaagtggatggcatttttaccgcggatccgcgcaaagtgccgagcgcgcgcctggtgccgattattaccccggatgaagcggcggaactgacctattatggcagcgaagtgattcatccgtttaccatggaacaggtgattcgcgcgcgcattccgattcgcattaaaaacgtggaaaacccgagcggcgcgggcaccgtgatttatccggatctgggctttccgcgcggcctggataccgaaccgccgaaagcggaacgcattgtggaaggcgtggatgaacgcatgccgaccgcggtgaccattaaagatgaaattattgtgctgaacattcatagcaaccgcaaaaccctgagccatggctttctggcgcgcatttttggcaccctggatcgcgcgggcgtggtggtggatctgattagcaccagcgaagtgcatgtgagcatggcgatgcaggattttctgaaccgcaaacgcctggaacgcctggtgaaagatctggaaaaaattggcgaagtgaccgtgagcaaagatatggcgattctgagcctggtgggccgcaacatgcgcaacgcgattggcagcgcgggcctgatgtttgcgagcctggcgcgcgcgatgattaacattgaaatgattagccagggcgcgagcgaaattaacattagctgcgtgattgaaaacaaagatgcgattaaagcgctgaacgtgattcatgaaagctgcctgagctatccgcgcagcccggcgaccgaaatggcgggcctgcagctgcag

SEQ ID NO: 54 - protein, 654 aa, Cryptococcus neoformans
MTRDFDSLFFTGPIFVDYSFTARHLESFLSSFPPQVSHAMSSASTPTAPYLEDLVRNSLDQTLPWVVQKYGGTSVGKSLDNITKIVGSYIDNGSKVAIVCSARSTQTKSLGTTNLLLQASREALQPALSSSGDGRSGSMSGTATPFYPKRVGSGFFGKDQSTSMVSSVSSLSQLEPQLGRSGSPSPFQSSSSRSPPRSPATPSQDSSVSQEPAFHATVDLIKKGHLEAARASLKEGPLRDELEEEIERDCESLRSFLYAAQIIDEISPRSQDSIVGTGERLACKIVAAALRDRGVDSELVVLDNIVDASMSAASEAISVDAGDQGVAQLGQEFYDQLSFRLGERLRECGQRVPVVTGYFGPVPGSLLAQIGRGYTDLCAALCAVGLKASELQVWKEVDGIFTADPRKVPSARLVPIITPDEAAELTYYGSEVIHPFTMEQVIRARIPIRIKNVENPSGAGTVIYPDLGFPRGLDTEPPKAERIVEGVDERMPTAVTIKDEIIVLNIHSNRKTLSHGFLARIFGTLDRAGVVVDLISTSEVHVSMAMQDFLNRKRLERLVKDLEKIGEVTVSKDMAILSLVGRNMRNAIGSAGLMFASLARAMINIEMISQGASEINISCVIENKDAIKALNVIHESCLSYPRSPATEMAGLQLQ

SEQ ID NO: 55 - DNA, 1269 bp, Cryptococcus neoformans
  source 1..1269: mol_type = genomic DNA
  CDS 1..1269
atgccgaaacagtggccgaccattgtgaaactggaaacctttattccgagcgcgcatggcagcggcggcgattatcatcgccagggcggcgatcattggattgtgcagggcaacattagctgcccgatgcataaatatgaagaatataaagtgagccgcaccagctggggcattggcgtgctgggcagcatttttgtgaaagtgcatgcgagcgatggcaccgtgggctatgcgaccggctttggcggcccgccggcgtgctggctgattgaagaacattttaaacgctttattgtgggccaggatccgcgcgataccaacaaaatgtgggatcagatgtttcgcgcgagcatgttttatggccgcaaaggcctgccgctggcggcgattagcgtggtggatctggcgatttgggatctgctgggcaaaattcgcggcgaaccgatttataaaatgattggcggccgcaccaaaaaagatattccgctgtatctgaccggcccgcgcccggaagtggcgaaaaaactgggcttttggggcagcaaagtggcgctgccgcatggcccgccggatggccatgaaggcattcgcaaaaacgtggaatatctgaaagcgtgcaaagaagcggtgggcccggattatccggtgcaggtggattgctatatgagcctggatgtgccgtataccattgcgctggtgaaagcgtgcgaaaaagcgggcgtggaaattaactggtgggaagaagtgctgcatccggatgattttgatggccatattaaactgaaagaagcgctgccgtatgtgaaatttaccaccggcgaacatgaatatagcaaatatggctttcgcaaactgattgaaaaccgcgcggtggatattattcagccggatgtgatgtggctgggcggcctgaccgaactgattaaagtggcggcgatggcggcggcgtatgatattccggtggtgccgcatggcagcggcccgtatagctttcaggcgattatgagctttccgaacagcgatttttgcgaatatattgcgaacagcccggatggcaaaagcattgaaccgagctttggcaacctgtttctgaacgaagtgctgccgcgcaacggccgcgtggatctgaccgatgaaccgggctttggcctggaactgaacccgagcgcggaactggtgccgtataaaagcttttttaccccgagcaaaagcctgggcgcggcgggcgaagtggaagatgatggcaaagcgaaagtgaacggcaaacat

SEQ ID NO: 56 - protein, 423 aa, Cryptococcus neoformans
MPKQWPTIVKLETFIPSAHGSGGDYHRQGGDHWIVQGNISCPMHKYEEYKVSRTSWGIGVLGSIFVKVHASDGTVGYATGFGGPPACWLIEEHFKRFIVGQDPRDTNKMWDQMFRASMFYGRKGLPLAAISVVDLAIWDLLGKIRGEPIYKMIGGRTKKDIPLYLTGPRPEVAKKLGFWGSKVALPHGPPDGHEGIRKNVEYLKACKEAVGPDYPVQVDCYMSLDVPYTIALVKACEKAGVEINWWEEVLHPDDFDGHIKLKEALPYVKFTTGEHEYSKYGFRKLIENRAVDIIQPDVMWLGGLTELIKVAAMAAAYDIPVVPHGSGPYSFQAIMSFPNSDFCEYIANSPDGKSIEPSFGNLFLNEVLPRNGRVDLTDEPGFGLELNPSAELVPYKSFFTPSKSLGAAGEVEDDGKAKVNGKH

SEQ ID NO: 57 - DNA, 1743 bp, Cryptococcus neoformans
  source 1..1743: mol_type = genomic DNA
  CDS 1..1743
atggcgaacgcgccgcatggcggcgtgctgaaagatctgctggtgcgcgatgcggcgctgcatgatagcctgctgcaggaagcgcgcagcctgaacgatatttttctgaccgaacgccagctgtgcgatctggaactgattctgaacggcggctttagcccgctggaaggctttatgaacgaacgcgattataccagcgtggtggaaaccctgcgcctggcgccgtataacggccagaaacatggcgatgtgtttccgattccgattaccctggatgtgagccaggaagatattaacaccctgggcctgaaacagggcggccgcgtggcgctgcgcgatccgcgcgatgatgcggcgctggcgattctgaccgtgagcgatatttatcgcccgaacaaagcgattgaagcggaaaaagtgatgggcgcggatgatattgcgcatccgagcgtggcgtatctgcgcaacaacgtgaaagaattttatgtgggcggcaaagtgcaggcgattcaggcgccgacccattttgattatgtgccgctgcgctttaccccggcggaactgcgcgcgcattttcataaactggcgtggcgcaaagtggtggcgtttcagacccgcaacccgatgcatcgcgcgcatcgcgaactgaccgtgcgcgcggcgcgccagcgccgcgcgaacgtgctgattcatccggtggtgggcctgaccaaaccgggcgatgtggatcattatacccgcgtgcgcgcgtatcaggcgctgatgccgagctatccggaaggcatggcgcatctggcgctgctgccgctggcgatgcgcatggcgggcccgcgcgaagcggtgtggcatgcggtgattcgcaaaaactttggcgcgacccattttattgtgggccgcgatcatgcgggcccgggcaaaaacagccagggccaggatttttatggcccgtatgatgcgcaggaactggtgacccagtttaaagatgaactgcagattgaaatggtgccgtttcaggcgatgacctatctgccgggcagcgatgaatatcagccggtggatgaagtgccgaaaggcaccccgaccgcggatattagcggcaccgaactgcgcaaacgcctgcgcaccggcgcgagcattccggattggtttagctataccggcgtggtgaaagtgctgcgcgaaagctatccgccgcgcccgcagcagggctttaccattctgctgaccggcctgcataacagcggcaaagataccattgcgcgcgcgctgcaggtgaccctgcagcagcagggcagccgcagcgtgagcctgctgctgggcgaagaactgcgcagcgatctggatccgcagattggccgcgcgattaccccggaacagaaacatattaacctggaacgcattggctttgtggcgggcgaactgaccaaagcgggcgcggcggtgattgcggcgccgaccgcgccgtatgaacgcagccgccaggcgtttaaaaaacaggtggtgggcagcggcggcggcaactattttctggtgcatgtggcgaccccgctggaatggtgcgaaaaagtggatcgccgcggcctgtataaagcggcgcgcgcgggcgaaattaaaaacctgaccggcgtggatgatgtgtatgaagcgccggaagatgcggatctggtgtgcgatctgcgcaacgataccgtgccggaaattgtgcatagcattattatgattctggaaagccagaacctggtg

SEQ ID NO: 58 - protein, 581 aa, Cryptococcus neoformans
MANAPHGGVLKDLLVRDAALHDSLLQEARSLNDIFLTERQLCDLELILNGGFSPLEGFMNERDYTSVVETLRLAPYNGQKHGDVFPIPITLDVSQEDINTLGLKQGGRVALRDPRDDAALAILTVSDIYRPNKAIEAEKVMGADDIAHPSVAYLRNNVKEFYVGGKVQAIQAPTHFDYVPLRFTPAELRAHFHKLAWRKVVAFQTRNPMHRAHRELTVRAARQRRANVLIHPVVGLTKPGDVDHYTRVRAYQALMPSYPEGMAHLALLPLAMRMAGPREAVWHAVIRKNFGATHFIVGRDHAGPGKNSQGQDFYGPYDAQELVTQFKDELQIEMVPFQAMTYLPGSDEYQPVDEVPKGTPTADISGTELRKRLRTGASIPDWFSYTGVVKVLRESYPPRPQQGFTILLTGLHNSGKDTIARALQVTLQQQGSRSVSLLLGEELRSDLDPQIGRAITPEQKHINLERIGFVAGELTKAGAAVIAAPTAPYERSRQAFKKQVVGSGGGNYFLVHVATPLEWCEKVDRRGLYKAARAGEIKNLTGVDDVYEAPEDADLVCDLRNDTVPEIVHSIIMILESQNLV

SEQ ID NO: 59 - DNA, 1716 bp, Cryptococcus neoformans
  source 1..1716: mol_type = genomic DNA
  CDS 1..1716
atgcacatcgcatacattctcggcctcgtacccctcgctttcgccggtgtcatcaagcacgaccctcccaagttccagcctattcagtctaccaggattgtgcggctgcacccaaacggggacaaaagcaagtgcgttgacctcctgggtaatactcgccaggatggtcagcccgtgcagatttgcgactgcgacggtaccccggctcaggactgggtcctcaatgccggccgcggtcagaccaaggtccagctcgccggcaccagtttctgtctcgatgccacccacccttacgcagccgacgggaccaacatgaagatctggaagtgcttggacgtccaacagcaagactggtattggacgagtgataacagaatcgttctccgcgaccagggcaagtgcctcgactgggccactggggatcggtctgatttcaaccagctgcaggtctggcggtgcagcacggataacaacaatcaggtctggacaacgggaccggactacggtgggaaccatgggggtgatgctggtgggaaccccggaggtaatcaaggcgatgattcaagaggcaaaaccaatactggtggaaaccccggaggtaatcaaggtggtgattcaggagggaaaaccaatcacatcattcccgaccccccagggccagaccccaacagcgagcccctcaaccccgcccttgaagccattgttaacgtcaccgaggcagctggaccctggccgcccatgatcaacttcgacggcgattacagcaatgacgacgtgaccgtatcggaccaagtaccgttcgactactgtatcggggaggggtctggtaacccgacggatgatgaaggacagcagcaaggacaaaacttcacagcaaatgtagctgggatagggagagacttctgcctggacaattttggcaatcctgacattcggaacaccatttctttcgacaacaacaccagcattgggaacggagcggacactgggcgagcccttcacaagcggacatttgcggattcaggggcgacgggtacgcccaaccggtggagacgagggtcggtgatttccatttgcgtcgagaggaacaacaattatctggttccatatgcgtcctcccccgttcccatccgagcatcggctatcgtcgcatccgccatggtacgtgcaatcaacttctggaacgcaggtctgaacaagcgattcgtctcgttcgagtttgtggagaactgcaacgacgccgtgttccatactcttgctgttgaccagatcaagtctgccaaagagcctactgtgctcgcgactgcccccttccctcctcggggtgaagagggtgctaggaaccgcaacatcttcgtgtggaatacggctttcgaggccaactttcagaacgtccttacctttatcatgtcacatgagctggggcacactcttggcctggcgcatgaggactgcaaatccagagaccaaccttgcgaagttatcactgacaaggtggctgggtcagtcgtggaaagccgtatctccggcagcaccacacagctgttcaatggccccaccccgcttgacatagcaggggcgaacgagtactactcacttgcagcgggacccaacaccccggagaacatcgtactctggcctgcgacgaggggtccgtttatcaactacccgccgctaccgaaatgcaagtggttcctcggtatttgctattactag

SEQ ID NO: 60 - protein, 571 aa, Cryptococcus neoformans
MHIAYILGLVPLAFAGVIKHDPPKFQPIQSTRIVRLHPNGDKSKCVDLLGNTRQDGQPVQICDCDGTPAQDWVLNAGRGQTKVQLAGTSFCLDATHPYAADGTNMKIWKCLDVQQQDWYWTSDNRIVLRDQGKCLDWATGDRSDFNQLQVWRCSTDNNNQVWTTGPDYGGNHGGDAGGNPGGNQGDDSRGKTNTGGNPGGNQGGDSGGKTNHIIPDPPGPDPNSEPLNPALEAIVNVTEAAGPWPPMINFDGDYSNDDVTVSDQVPFDYCIGEGSGNPTDDEGQQQGQNFTANVAGIGRDFCLDNFGNPDIRNTISFDNNTSIGNGADTGRALHKRTFADSGATGTPNRWRRGSVISICVERNNNYLVPYASSPVPIRASAIVASAMVRAINFWNAGLNKRFVSFEFVENCNDAVFHTLAVDQIKSAKEPTVLATAPFPPRGEEGARNRNIFVWNTAFEANFQNVLTFIMSHELGHTLGLAHEDCKSRDQPCEVITDKVAGSVVESRISGSTTQLFNGPTPLDIAGANEYYSLAAGPNTPENIVLWPATRGPFINYPPLPKCKWFLGICYY

SEQ ID NO: 61 - DNA, 1500 bp, Cryptococcus neoformans
  source 1..1500: mol_type = genomic DNA
  CDS 1..1500
atgccgagcctgacccagaccaaagatctggcgagcctgctgagcgatgcgagccattttaaacagaaaggctatattaacggcgaatgggtgagcgcgagcgatggcgcgacctttccgctgtataacccggcgaccggcgcgaaactggcggatatgccgcatatgccgcgcagccaggtggcggaagcgattaacgcggcgaaagcggcgtttccggcgtgggcggcgctgaccgcgtatcagcgccagaactatctgctgaaactgtttaaagaaatggaagaacatagcgaagatctggcgattattctgtgcaccgaaaacggcaaaccgctggcggaaagccgcgtggaaattagctatggcgcgagctttctgacctggaacgcggcggaagcgctgcgcacctatggccagaccattccgagcccgtttccgggcacccgcaacaccgtgattaaacagccgattggcgtgtgcggcctgattaccccgtggaactttccgaacgcgatgattacccgcaaaatggcgccggcgctggcggcgggctgcaccgtggtgattaaagcgccggcggaaaccccgctgagcgcgctggcgatgtgcgtgctgtgcgaacgcgtgggcattccgccgggcgtggtgaacgtggtgaccatggataaaggccagcgcgaaatggcggcgggcctggaactgtgcgaaaacgtgaaagtgagcaaaattagctttaccggcagcaccccggtgggccgcctgctgatgaaacagagcagcggcaccctgaaaaaactgagctttgaactgggcggcaacgcggcgtttattatttttgatgatgcggatctggatctggcggtgaacggcgtgattctgagcaaatttcgcgcggcgggccagacctgcatttgcgcgaaccgcatttttgtgcatagcaaaatttatgatgattttgcgcgccgcctggtggaacgcgtgaaagcgtttaaagtgggcaacggcattgaagaaggcgtgaccattggcccgctggtgagccagcgcggcgtggaaaaagtggaacgccatgtgcaggatgcggtgggcctgggcgcgaaagtgctggtgggcggcaaacgcattgataaaggcgaaggcagctgcttttatgaaccgaccgtgctggtggatgtgccgcgccagtgcgcggtgagcaacgaagaaacctttggcccgctggcgccgctgtttaaatttgatgatgaagatgatgtggtggaacgcgcgaacagcagcgaagtgggcctggcggcgtatttttttaccaaagatctggcgcgcacccatcgcgtggcggaaaaactggaagtgggcatggtggcggtgaacaccggcgcgattgcgcagagctgcgtgccgtttggcggcgtgaaacagagcggctttggccgcgaaggcggcccgagcggcattgatgaatttatggtggaaaaactgattaccattggcggcctg

SEQ ID NO: 62 - protein, 500 aa, Cryptococcus neoformans
MPSLTQTKDLASLLSDASHFKQKGYINGEWVSASDGATFPLYNPATGAKLADMPHMPRSQVAEAINAAKAAFPAWAALTAYQRQNYLLKLFKEMEEHSEDLAIILCTENGKPLAESRVEISYGASFLTWNAAEALRTYGQTIPSPFPGTRNTVIKQPIGVCGLITPWNFPNAMITRKMAPALAAGCTVVIKAPAETPLSALAMCVLCERVGIPPGVVNVVTMDKGQREMAAGLELCENVKVSKISFTGSTPVGRLLMKQSSGTLKKLSFELGGNAAFIIFDDADLDLAVNGVILSKFRAAGQTCICANRIFVHSKIYDDFARRLVERVKAFKVGNGIEEGVTIGPLVSQRGVEKVERHVQDAVGLGAKVLVGGKRIDKGEGSCFYEPTVLVDVPRQCAVSNEETFGPLAPLFKFDDEDDVVERANSSEVGLAAYFFTKDLARTHRVAEKLEVGMVAVNTGAIAQSCVPFGGVKQSGFGREGGPSGIDEFMVEKLITIGGL

SEQ ID NO: 63 - DNA, 1200 bp, Cryptococcus neoformans
  source 1..1200: mol_type = genomic DNA
  CDS 1..1200
atgtgcagcagccatgcgaccgcggtggaaagcgtgagcccggcgccgcgcaaaagccagtatgaagtgaaatatgatccggatctggtgctgaaaagcgcggaatttaaagaactgaaacagggcgataaagaactggaagatccgaaagcgaacctggcgtgcgcgtatgatgaaaaacataacgtgaaaatgattaacaaaccgattccgaaagcgcgccaggatgaagtggtggtgcatattaaagcgaccggcatttgcggcagcgatgtgcatttttggaaacatggccagattggcccgaccatgattgtgaccgatacctgcggcgcgggccatgaaagcgcgggcgaagtggtggaagtgggcccgggcgtggaacagtggaaagtgggcgatcgcgtggcgattgaatgcggcgtgccgtgcggccaggcgagctgcggcccgtgcgtgaccggccgctataacgcgtgcccgcaggtggtgttttttagcaccccgccgtatcatggcaccctgacccgctatcatgcgcatccggcgagctggctgcatcgcctgccggataacctgagctatgaagaaggcgcgctgtgcgaaccgtttgcggtggcgctggcggcgctggaacgcgcgggcaaccgcctgggcgatccggtgctgatttgcggcgcgggcccgattggcctggtgaccctgctggcgagccatgcggcgggctgcaccccgattgtgattaccgatctgcaggcgagccgcctggaagtggcgaaaaaactgattccgaccgtgaaaaccgtgcagattgaacgcagctggaccagcaaagaaaccagcgaagcgattaaagaagcggcgggcaccggcattcgcgtggcgattgatgcgaccggctttgaaagcagcattaccgcggcgatttatagcgtggtgtttggcggcaaagtgtttgtgattggcgcgggcccgagcgaacagaaatatccgtttggctattgcagcgcgaacgaaattgatctgcagtttcagtatcgctatgcgcatcagtatccgaaagcgctgcgcattgtgagcggcggcctgattaacctgaaaccgctgctgacccatacctttccgctgaacaaagcggtggaagcgtttcatgtggcggcggatccgaccaaaggcgcgattaaagtgcagattattgat

SEQ ID NO: 64 - protein, 400 aa, Cryptococcus neoformans
MCSSHATAVESVSPAPRKSQYEVKYDPDLVLKSAEFKELKQGDKELEDPKANLACAYDEKHNVKMINKPIPKARQDEVVVHIKATGICGSDVHFWKHGQIGPTMIVTDTCGAGHESAGEVVEVGPGVEQWKVGDRVAIECGVPCGQASCGPCVTGRYNACPQVVFFSTPPYHGTLTRYHAHPASWLHRLPDNLSYEEGALCEPFAVALAALERAGNRLGDPVLICGAGPIGLVTLLASHAAGCTPIVITDLQASRLEVAKKLIPTVKTVQIERSWTSKETSEAIKEAAGTGIRVAIDATGFESSITAAIYSVVFGGKVFVIGAGPSEQKYPFGYCSANEIDLQFQYRYAHQYPKALRIVSGGLINLKPLLTHTFPLNKAVEAFHVAADPTKGAIKVQIID

SEQ ID NO: 65 - DNA, 1689 bp, Cryptococcus neoformans
  source 1..1689: mol_type = genomic DNA
  CDS 1..1689
atggtgtgcgtgattagcgatccggattggtggcgccaggcggtggtgtatcagatttatccgcgcagctttgcggatgcgaacggcgatggcattggcgatctgaaaggcattaccgcgcgcgtgccgtatctgaaagcgctgggcgtggatgcgatttggctgagcccgttttatccgagcgcgctgcgcgatggcggctatgatgtggcggattatcgcgatgtggatccgaaaattggcaccctggaagaatttgatgaaatgaccgcggcgtttcagaaagtgggcattcgcgtgattgtggatattgtgccgaaccatagcagcgatgatcatgaatggtttcaggcggcgctgaaagcgggcaaaggcagcccggaacgcgaacgctatatttttcgcgatggcctgggcccgaacaaagatcagccgccgaccgattggatttgcagctttggcggcagcgcgtggagcccgagcggcatgaacgatggccagtggtattttcattggtttgatagcagccagccggattggaactgggaaaacccggatgtgaaagcggattttctgaaaaccctgaaattttggggcgatcgcggcgtgagcggctttcgcattgatgtggcgcatggcctggcgaaagatatgagcgaaccgctgccgaactgggaacagctgaccaaactgacccatcagaaactgaccaacggcaacagcgaactggatcatccgctgctggatcgcaaagaagtgcatgatatttatcgcagctggcgcgaagtgtttaaccagtttaacccgccgctgatggcggtggcggaagcgtgggtggcgccggatcagaaaccgctgtatgcgagcagcgaaggcctgggccagacctttagctttgatattctgctgtgcaactttgatgcggaagaatatcgccagtgcattaaaagcagcctggcgggcagcaaaaaaagcgatagcaccaccacctgggtgctgagcaaccatgatgtgatgcgccatccgacccgctttggcctgccgaacgtgccgaacgcgaaccatgcgatgaccaccgatacctataacaaatttctgaaaaccaaactgaccgatccgaaagtggatattgaacagggcctgcgccgcgcgaaagcggcgaccctgatgattctggcgctgccgggcagcacctatctgtatcagggcgaagaactgggcctgcaggaagtggtggaaattccggatgaagaacgccaggatccgatttttattcgcaccaaaggcgaagaagtgggccgcgatggctgccgcgtgccgattccgtgggtggcggatgaaaaaaactttggctatggcccgggcaaacgcgcgcatctgccgcagccggcgtggtttaaagattatgcggtggatgtggaagaaaaagatgcgaacagcgtgctgagcctgtatcgccgcgcgctgggcctgcgcaaaggcctgcagagcgcggaagaactggaatgggtggaaaacccgaacaaagaagtgctgcattttcgccgcccgggcggctgggaagtggtggtgaacattggcaaagatagcgtggatctgccgaaaggcagcgtgctgattagcagcagcaacaacgcgctgaaaggcggcagcattccgggcgaaaccaccgtgtggctgaaaagcgcg

SEQ ID NO: 66 - protein, 563 aa, Cryptococcus neoformans
MVCVISDPDWWRQAVVYQIYPRSFADANGDGIGDLKGITARVPYLKALGVDAIWLSPFYPSALRDGGYDVADYRDVDPKIGTLEEFDEMTAAFQKVGIRVIVDIVPNHSSDDHEWFQAALKAGKGSPERERYIFRDGLGPNKDQPPTDWICSFGGSAWSPSGMNDGQWYFHWFDSSQPDWNWENPDVKADFLKTLKFWGDRGVSGFRIDVAHGLAKDMSEPLPNWEQLTKLTHQKLTNGNSELDHPLLDRKEVHDIYRSWREVFNQFNPPLMAVAEAWVAPDQKPLYASSEGLGQTFSFDILLCNFDAEEYRQCIKSSLAGSKKSDSTTTWVLSNHDVMRHPTRFGLPNVPNANHAMTTDTYNKFLKTKLTDPKVDIEQGLRRAKAATLMILALPGSTYLYQGEELGLQEVVEIPDEERQDPIFIRTKGEEVGRDGCRVPIPWVADEKNFGYGPGKRAHLPQPAWFKDYAVDVEEKDANSVLSLYRRALGLRKGLQSAEELEWVENPNKEVLHFRRPGGWEVVVNIGKDSVDLPKGSVLISSSNNALKGGSIPGETTVWLKSA

SEQ ID NO: 67 - DNA, 729 bp, Cryptococcus neoformans
  source 1..729: mol_type = genomic DNA
  CDS 1..729
atgccgagcgtggtgtttgatgtggtgggcacctgctttagctatgataacggcgcggaagcgctgcaggcgcgcctgggcccgaaactggcgaaatatggcattccgagcaaactgctgttttatagctgggtgtgcagcaccgaacgcgattatagctatctgagccagattaaacagtataaagcgttttttgcgattctgagcaacaccctgacccgcgtgctgtttcaggcgggcgtgccggtggaagcgctggatgatttttttaccgcggatgatgtggattatattatgaacgaatataaaaaactgaaagcgcgcccgggcctggcggaaatgatgcagaccctgcgcgatggcggctttgaagtgtggtgctgcagcgatgcgaacgtggatcgcgtgaaaggctattttgataacgcgggcgtggaaatgccgctggatcatattctgagcgcggatatggtgaaagcgggcaaaccggaagcggcggtgtataaatttgcgcgcgaaaaagcgggcagcgatcagccgggcgaagtgagcgtgtttgcggcgagccatgcgtgggattgcgcggcggcgaaagcggcgggctttctgaccgcgtataccaccacctatgaatatgatgaatgcgaagtgatttttggcaaaagcgatctggtggcgccggatctggtgagcctgggcaaaggcattgtggaaaaatggggcaaaaaa

SEQ ID NO: 68 - protein, 243 aa, Cryptococcus neoformans
MPSVVFDVVGTCFSYDNGAEALQARLGPKLAKYGIPSKLLFYSWVCSTERDYSYLSQIKQYKAFFAILSNTLTRVLFQAGVPVEALDDFFTADDVDYIMNEYKKLKARPGLAEMMQTLRDGGFEVWCCSDANVDRVKGYFDNAGVEMPLDHILSADMVKAGKPEAAVYKFAREKAGSDQPGEVSVFAASHAWDCAAAKAAGFLTAYTTTYEYDECEVIFGKSDLVAPDLVSLGKGIVEKWGKK

(19) United States
(12) Patent Application Publication
(10) Pub. No.: US 2024/0247299 A1
(43) Pub. Date: Jul. 25, 2024

(54) METHOD TO SCREEN COMPOUNDS FOR ANTIFUNGAL ACTIVITY AND PHARMACEUTICAL COMPOSITIONS AND METHODS TO TREAT FUNGAL DISEASES BY INHIBITING SPORE GERMINATION

(71) Applicant: Wisconsin Alumni Research Foundation, Madison, WI (US)

(72) Inventors: Christina Marie HULL, Middleton, WI (US); Mingwei HUANG, Madison, WI (US); Sebastien Claude Ortiz, Madison, WI (US)

(73) Assignee: Wisconsin Alumni Research Foundation, Madison, WI (US)

(21) Appl. No.: 18/631,396

(22) Filed: Apr. 10, 2024

Related U.S. Application Data
(63) Continuation of application No. 16/369,939, filed on Mar. 29, 2019, now Pat. No. 11,981,953.
(60) Provisional application No. 62/649,802, filed on Mar. 29, 2018.

(51) Int. Cl. C12Q 1/18; A01N 33/10; A01N 37/44; A01N 41/12; A01N 47/40; A61K 31/137; A61K 31/145; A61K 31/155; A61K 31/24; A61P 31/10; C12Q 1/66

(52) U.S. Cl. CPC C12Q 1/18; A01N 33/10; A01N 37/44; A01N 41/12; A01N 47/40; A61K 31/137; A61K 31/145; A61K 31/155; A61K 31/24; A61P 31/10; C12Q 1/66; G01N 2333/375

(57) ABSTRACT

A method of testing compounds for activity to inhibit germination of spores. The method includes the steps of (a) providing bacterial, fungal, or plant spores transformed to contain and express a detectable marker, wherein the marker when expressed, is operationally linked to a spore-specific or yeast-specific protein, in a medium and under environmental conditions in which the spores will germinate, and measuring a first signal output generated by the marker prior to the spores initiating germination; (b) contacting the spores of step (a) with a compound whose activity to inhibit germination of spores is to be measured; (c) incubating the spores of step (b) under environmental conditions and for a time wherein spores not treated with the compound will germinate; and (d) determining extent of germination of the spores by measuring a second signal output generated by the marker, wherein a difference between the first signal output and the second signal output is proportional to the extent of germination of the spores. Also described are compositions of matter for inhibiting spore germination in vitro and in vivo.

### CROSS-REFERENCE TO RELATED APPLICATIONS

The present application is a continuation of co-pending U.S. application Ser. No. 16/369,939, filed Mar. 29, 2019, which claims benefit of priority to U.S. provisional application Ser. No. 62/649,802, filed Mar. 29, 2018, both of which are incorporated herein by reference.

### FEDERAL FUNDING STATEMENT

This invention was made with government support under AI089370 awarded by the National Institutes of Health. The government has certain rights in the invention.

### SEQUENCE LISTING

The instant application contains a Sequence Listing which has been submitted in an XML file with the USPTO through Patent Center and is hereby incorporated by reference in its entirety. The Sequence Listing XML, created on Mar. 26, 2024, is named “SEQ_LIST-09824507-P180117US03.xml” and is 124,578 bytes in size.

### BACKGROUND

Spores are an essential cell type required for long-term survival across diverse organisms and are a hallmark of fungal reproduction, persistence, and dispersal. Among human fungal pathogens, spores are presumed infectious particles, but relatively little is known about this robust cell type. Sporulation enables a relative quiescence—a type of hibernation—that contributes to the survival of fungi. However, sporulation also requires a transition back into a vegetative form so that the fungi can replicate—i.e., germination. Germination, despite its central importance in fungal reproduction and pathology in plants and animals, is not well understood.

Spores are a particularly successful cell type used by many microorganisms, including bacteria, fungi, and protozoa to survive unsuitable growth conditions and/or to disperse to new environments. Among eukaryotes, some of the most environmentally resistant spores are those of fungi, and much of our current understanding of spores comes from studies in model fungi such as Saccharomyces cerevisiae and Aspergillus nidulans. There are two general categories of fungal spores—sexual and asexual, and both forms occur across diverse fungal species via myriad developmental strategies. For example, in the budding yeast S. cerevisiae sexual spores are formed when yeast diploids are subject to nitrogen starvation and a non-fermentable carbon source, resulting in four haploid ascospores; S. cerevisiae does not produce asexual spores. In contrast, the filamentous fungus Aspergillus nidulans produces both asexual and sexual spores via the development of multicellular fruiting structures with thousands of spores per structure. In all instances, however, spores are adapted for general survivability.

As a consequence, fungal spores share three basic characteristics: First, mature spores are relatively metabolically quiescent, allowing them to remain dormant for long periods of time under sub-optimal growth conditions (e.g. in the absence of nutrients). Second, spores are resistant to environmental stresses, such as high temperatures, desiccation, and UV radiation, thus facilitating long-term survival and/or dispersal across great distances. Third, upon encountering growth-promoting environments, spores rapidly escape quiescence and germinate to resume vegetative growth. As a result, fungi are ubiquitous across all ecosystems on earth.

Spore-producing fungi commonly generate spores with thick, protective coats and robust stress resistance. Spores respond to different environmental signals to initiate germination, depending on their adapted niches. For example, spores of S. cerevisiae germinate readily in response to the presence of a fermentable carbon source. In contrast, spores of Talaromyces macrosporus require nutrients and a rigorous external trigger of very high temperature or pressure. These triggers generally result in responses such as water uptake, cell wall remodeling, and activation of nutrient metabolism and protein synthesis, leading to active fungal growth.

The transition from dormant particle to actively growing cell is particularly important because fungal survival cannot occur in the absence of the ability to germinate when (and only when) appropriate for vegetative growth. Environmental fungi are well adapted to their niches, and interestingly, these adaptations have led to a handful of fungi with the ability to cause life-threatening diseases in humans. Histoplasma capsulatum, Blastomyces dermatitidis, Aspergillus fumigatus, Coccidioides immitis, Sporothrix schenkii, Penicillium marneffei, and Cryptococcus neoformans are the most common environmental fungi that can cause disease in humans. The general route of infection is by inhaling cells from environmental sources. Spores (sexual or asexual, depending on the fungus) are the most likely infectious particles for all of these pathogens; however, very little is known about their basic spore biology, making the development of disease prevention and treatment strategies challenging.

Among human fungal pathogens, the most common cause of fatal fungal disease (and a well-developed model for study) is Cryptococcus neoformans, a primarily opportunistic pathogenic yeast that causes meningoencephalitis. People with AIDS are particularly susceptible, and there are an over 200,000 cases and nearly as many deaths annually worldwide from cryptococcosis. Rajasingjam R, Smith R M, Park B J, Jarvis J M, Govender N, Chiller T M, Denning D W, Loyse A, Boulware D R (2017) “Global burden of disease of HIV-associated cryptococcal meningitis: and updated analysis” Lancet Infectious Disease 17: 873-881 (pmid:2848341). C. neoformans is ubiquitous in the environment, and inhalation of aerosolized spores and/or yeast is the most common route of infection of humans. Under laboratory conditions, spores are produced through sexual development between haploid yeast of opposite mating types (a and a) or by a fruiting. In response to specific environmental conditions, cells form filaments and fruiting bodies (basidia) from which haploid, recombinant spores bud in chains.

Spores of C. neoformans exhibit the fundamental properties of most fungal spores, such as stability in the absence of nutrients and resistance to a variety of environmental stresses, including high temperature, desiccation, and oxidative stress. These spores have also been shown to germinate efficiently and synchronously in response to nutrients, and they germinate and cause disease in a mouse inhalation model of infection. See Velagapudi R, Hsuch Y-P, Geunes-Boyer S, Wright J R, Heitman J (2009) “Spores as infectious propagules of Cryptococcus neoformans,” Infect Immun. 77:4345-4355 (pmid: 19620339) and Giles S S, Dagenais T R T, Botts M R, Keller N P, Hull C M (2009) “Elucidating the pathogenesis of spores from the human fungal pathogen Cryptococcus neoformans,” Infect Immun 77:3491-3500 (pmid: 19451235). These findings indicate that C. neoformans spores harbor intrinsic properties that facilitate survival in the environment, maintain spore viability and stability, and initiate germination in response to external signals, including those of a mammalian host.

Current antifungal therapeutics are relatively limited because of high toxicity or insufficient efficacy. These issues arise because, unlike bacteria, fungi are eukaryotes. Thus, fungi are far more similar (metabolically and biochemically) to plants and animals than are bacteria. In short, compounds that interfere with fungal biology or are toxic to fungi, tend also to interfere with or be toxic to humans and animals.

A comparatively small number of antifungal compounds are approved for human, veterinary, and agricultural use in the United States. Focusing on antifungal drugs approved for use in humans, the gold standard by which all other antifungal pharmaceuticals are measured in terms of systemic antifungal activity is the polyene amphotericin B, first marketed in 1955. It is widely used to treat life-threatening fungal infections such as invasive mucormycosis, cryptococcal meningitis, aspergillosis, and candidiasis. While highly effective against fungi, amphotericin B itself has a slew of well-known and potentially life-threatening side effects. When administered intravenously, amphotericin B typically induces a debilitating set of symptoms, including high fever, shaking chills, hypotension, anorexia, nausea, vomiting, headache, dyspnea and tachypnea, drowsiness, and generalized weakness. Kidney damage is a commonly reported side effect. As a result, amphotericin B is administered with very close monitoring of the patient by healthcare professionals.

Other antifungal compounds approved for use in humans include imidazoles (e.g., miconazole), triazoles (e.g., fluconazole), and thiazole antifungals (e.g., abafungin). Most of these types of antifungal compounds, however, are used topically, rather than systemically. They are much less toxic that amphotericin B, but not as efficacious.

Echinocandins are a much newer class of systemic antifungal compounds approved for use in humans. The echinocandins are macrocyclic lipopeptides. Their structure is characterized by (typically) a 6-mer macrocyclic peptoid moiety bonded to a long (e.g., >C10) hydrocarbon tail. Echinocandins inhibit the synthesis of glucan in the cell wall of fungi via noncompetitive inhibition of the enzyme 1,3-β glucan synthase. In this sense, they exert a pharmacological activity against fungi that is analogous to the pharmacological activity of beta-lactam antibiotics against bacteria. Echinocandins are also far less toxic than amphotericin B, but again, not as effective.

Thus, there remains a long-felt and unmet need for a method to test new and existing compounds for their ability to inhibit fungal growth.

### SUMMARY

While vegetative fungi are similar metabolically and biochemically to other eukaryotic cells, fungi also sporulate and germinate. Thus, chemical inhibitors of fungal germination are potentially highly useful compounds in antifungal compositions (i.e., human and veterinary pharmaceuticals, topical and systemic pharmaceuticals, and agricultural and industrial fungicides). Thus, disclosed herein is a fluorescence-based quantitative germination assay suitable for high throughput screening. Using the subject germination assay, a screening of a 75,000-compound library yielded 108 germination-inhibiting compounds. Some of these compounds exhibited specific activity to inhibit germination of Cryptococcus spores (as contrasted to inhibiting vegetative cell growth). This indicates that germination itself is an effective target in developing antifungal drugs for prophylactic use in at-risk patients.

Thus, disclosed herein is a method of testing compounds for activity to inhibit germination of spores. The method comprises providing bacterial, fungal, or plant spores transformed to contain and express a detectable marker, wherein the marker is operationally linked to a spore-specific or yeast-specific protein, in a medium and under environmental conditions in which the spores will germinate, and measuring a first signal output generated by the marker prior to the spores initiating germination. The spores are then contacted with a compound whose activity to inhibit germination of spores is to be measured. The spores are then incubated under environmental conditions and for a time wherein spores not treated with the compound will germinate. The extent of germination of the spores is determined by measuring a second signal output generated by the marker, wherein a difference between the first signal output and the second signal output is proportional to the extent of germination of the spores.

In certain versions of the method, the marker is operationally linked to a spore-specific protein selected from the group consisting of XP_567740.1 (SEQ. ID. NO: 2), XP_566791.1 (SEQ. ID. NO: 4), XP_570303.1 (SEQ. ID. NO: 6), XP_571089.1 (SEQ. ID. NO: 8), XP_571997.1 (SEQ. ID. NO: 10), XP_569295.1 (SEQ. ID. NO: 12), XP_569173.1 (SEQ. ID. NO: 14), XP_569068.1 (SEQ. ID. NO: 16), XP_569336.1 (SEQ. ID. NO: 18), XP_567136.1 (SEQ. ID. NO: 20), XP_568990.1 (SEQ. ID. NO: 22), XP_570610.1 (SEQ. ID. NO: 24), XP_571921.1 (SEQ. ID. NO: 26), XP_572925.1 (SEQ. ID. NO: 28), XP_570796.1 (SEQ. ID. NO: 30), XP_571548.1 (SEQ. ID. NO: 32), XP_570447.1 (SEQ. ID. NO: 34), and XP_571343.1 (SEQ. ID. NO: 36).

Another version of the method comprises the steps described previously, and further comprising plotting the area and aspect ratio of the spores and any germinated cells after the incubation of step (c). Because spores tend to be smaller and have a more oblong aspect ratio than do germinated, vegetative cells, the extent of germination can be determined by measuring the distribution of the cells' area versus aspect ratio. Again, in this version of the method, the marker, if present, is operationally linked to a spore-specific protein selected from the group consisting of XP_567740.1 (SEQ. ID. NO: 2), XP_566791.1 (SEQ. ID. NO: 4), XP_570303.1 (SEQ. ID. NO: 6), XP_571089.1 (SEQ. ID. NO: 8), XP_571997.1 (SEQ. ID. NO: 10), XP_569295.1 (SEQ. ID. NO: 12), XP_569173.1 (SEQ. ID. NO: 14), XP_569068.1 (SEQ. ID. NO: 16), XP_569336.1 (SEQ. ID. NO: 18), XP_567136.1 (SEQ. ID. NO: 20), XP_568990.1 (SEQ. ID. NO: 22), XP_570610.1 (SEQ. ID. NO: 24), XP_571921.1 (SEQ. ID. NO: 26), XP_572925.1 (SEQ. ID. NO: 28), XP_570796.1 (SEQ. ID. NO: 30), XP_571548.1 (SEQ. ID. NO: 32), XP_570447.1 (SEQ. ID. NO: 34), and XP_571343.1 (SEQ. ID. NO: 36),

Also disclosed herein are antifungal compositions and method of using them as topical and systemic fungicides for industrial, agricultural, and pharmaceutical uses. Disclosed herein is a composition of matter for inhibiting germination of fungal spores, the composition comprising a spore germination-inhibiting concentration of a compound selected from the group consisting of

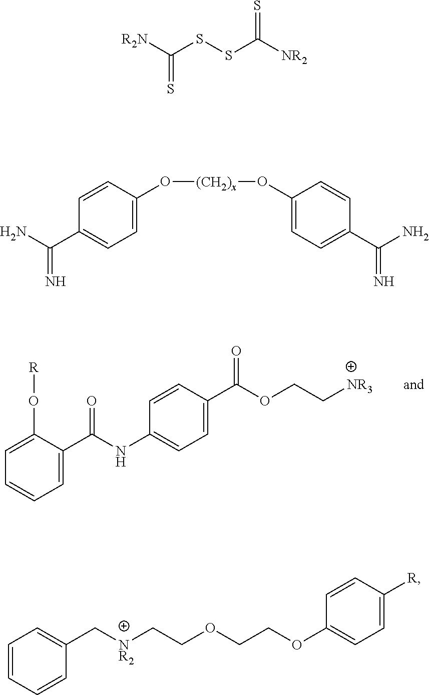

and salts thereof, in combination with a vehicle.

Also disclosed herein is a pharmaceutical composition for inhibiting fungal infection in mammals (as well as the corresponding method of inhibiting topical or systemic fungal infections in mammals, including humans), the composition comprising a spore germination-inhibiting amount of a compound selected from the group consisting of:

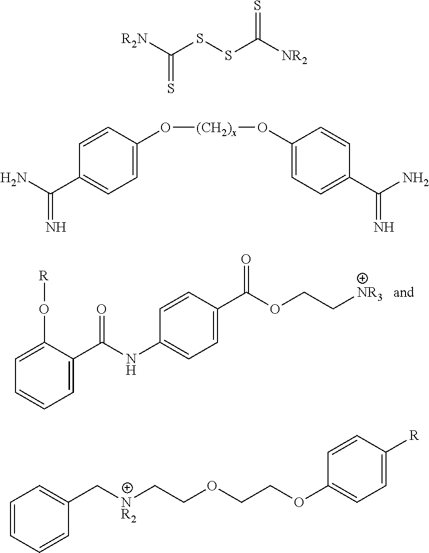

wherein R is linear or branched C₁₋₁₂ alkyl and “x” is an integer of from 1 to 12, and salts thereof, in combination with a pharmaceutically suitable vehicle.

### BRIEF DESCRIPTION OF THE DRAWINGS

FIG. 1 is a graph showing that C. neoformans var. grubii spores are pathogenic in a murine model of cryptococcosis. Groups of eight AJ/Cr mice were infected with C. neoformans var. grubii spores (10⁵) or yeast (10⁵) via intranasal inhalation. See Giles et al. Infect. Immun. 2009, 77(8):3491. Time post-infection (in days) is shown in the X-axis; percent of surviving mice is shown on the Y-axis.

FIG. 2A is a series of scanning electron micrographs showing morphological transitions during germination. The germinating spore is false-colored green; the emerging yeast wall is false-colored yellow, and the resulting daughter cell is false-colored orange. Bar=1 μm for 0, 4, and 8 Hr; bar=2 μm for 12 Hr.

FIG. 2B is a series of micrographs analogous to those in FIG. 2A using transmission electron microscopy rather than scanning electron microscopy. Bars=500 nm.

FIG. 3A is a schematic diagram of a screening assay for an uninhibited germination reporter strain as it undergoes germination (left-hand panel) and graphs depicting germination as reported by NanoLuciferase (NL) activity (top right, NanoLuc®-brand luciferase, Promega Corporation, Madison, WI), and as measured by optical density (OD) (bottom right). The schematic pictures below show the morphology and number of cells over time. This example depicts a yeast-specific protein.

FIG. 3B is a schematic diagram showing the workflow for the screening assay.

FIG. 3C shows representative plates from the screening assay described in FIGS. 3A and 3B, showing wells that contain germination-inhibiting compounds in red.

FIG. 3D is a schematic diagram of the transformation construct containing a marker, in this case a gene encoding luciferase (“Nano-Luc”).

FIG. 4 is a flow chart showing the workflow of a high-throughput screening assay according to the present disclosure.

FIG. 5A shows schematic views of a microliter-scale well device and how it operates. The microfluidic device includes an input port connected to a culture well connected to an output port. Each microfluidic chamber is built upon a transparent support, such as a glass microscope slide. The microfluidic device is dimensioned and configured to culture and image non-adherent cells, such as spores and germinated fungi, yeast, and the like. The top panel of FIG. 5A shows a perspective view of a single microfluidic culturing device. The middle panel of FIG. 5A shows six (6) such devices disposed on a glass slide. Each of the six devices shown is filled with 10 μL of blue dye. The bottom panel of FIG. 5A shows a front elevation cutaway of the device shown in the top panel.

FIG. 5B depicts representative raw images of the fungal cells (spores and germinated cells) in the device shown in FIG. 5A. Image processing steps are then applied to the raw images to discriminate between spores and germinated cells. These process steps may include, without limitation, applying a density threshold to the raw images and then automatically detecting and measuring the cells 2-D area and aspect ratio.

FIG. 6 depicts how photos of germinating spores in the micro-device depicted in FIG. 5A were analyzed for the size and shape of the cells and the aspect ratio calculated. This was done using modified algorithms of ImageJ, a public domain, open-source, Java-based image processing program, which was developed originally by Wayne Rasband at the Research Services Branch of the National Institutes of Health. ImageJ can be downloaded free of charge at https://imagej.nih.gov/ij/download.html. The images of the cells are then plotted based on their 2-D area (X-axis) versus their aspect ratio (Y-axis) as shown in the right-hand panels of FIG. 6. Spores, because they are more oblong and smaller in area, plot to the bottom left-hand side of the histogram; germinated cells, because they are more spherical and larger in area, plot to the upper right quadrant of the plots.

FIG. 7A depicts a series of photographic analyses showing that germination in microscale devices as described herein can be determined by cell area versus aspect ratio. Thus, each panel in FIG. 7A depicts the germination dynamics of spores visualized by 2D histograms of cell area vs. aspect ratio. Data are also shown as a stacked bar plot of the population composition over time (at lower right). Colors are normalized on each plot such that yellow represents the area and aspect ratio combination with the most cells observed and dark blue represents area and aspect ratio combinations that were not observed. Cells in the lower left quadrant are defined as spores; cells in the upper right quadrant as yeast; all remaining cells are classified as intermediates.

FIG. 7B shows 2D histograms as in FIG. 7A, but for a 16-hour germination of Cryptococcus spores using PBS as a control (no germination), synthetic dextrose growth medium (SD) alone (full germination in the absence of compounds), and fluconazole (16 mg/mL) in the presence of growth medium.

FIGS. 8A and 8B show that pentamidine treatment lowers fungal burden in mouse lung. FIG. 8A is a graph showing lung colony-forming units quantified for each mouse infected with JEC20×JEC21 yeast. The test group of mice were treated with 4 mg/kg/day pentamidine; the control group of mice were treated with 1×PBS; * p<0.05 for two-tailed paired t-test.

FIG. 8B is a graph depicting lung colony-forming units quantified for each mouse infected with JEC20×JEC21. Again, the test mice were treated with 4 mg/kg/day pentamidine; the control mice were given 1×PBS; ** p<0.01 for two-tailed paired t-test.

FIG. 9 is a graph showing that pentamidine prophylactically inhibits fungal spore germination in vivo. FIG. 9 depicts lung colony-forming units quantified for each mouse infected with JEC20×JEC21 spores. The test group of mice were treated with 4 mg/kg/day pentamidine; the control group of mice were treated with 1×PBS. ** p<0.01 for two-tailed paired t-test. See Examples for complete details.

### DETAILED DESCRIPTION

### Abbreviations and Definitions

The term “pharmaceutically-suitable salt” refers to any acid or base addition salt whose counter-ions are non-toxic to the patient in pharmaceutical doses of the salts, so that the beneficial inhibitory effects inherent in the free base or free acid are not vitiated by side effects ascribable to the counter-ions. A host of pharmaceutically-suitable salts are well known in the art. For basic active ingredients, all acid addition salts are useful as sources of the free base form even if the particular salt, per se, is desired only as an intermediate product as, for example, when the salt is formed only for purposes of purification, and identification, or when it is used as intermediate in preparing a pharmaceutically-suitable salt by ion exchange procedures. Pharmaceutically-suitable salts include, without limitation, those derived from mineral acids and organic acids, explicitly including hydrohalides, e.g., hydrochlorides and hydrobromides, sulphates, phosphates, nitrates, sulphamates, acetates, citrates, lactates, tartrates, malonates, oxalates, salicylates, propionates, succinates, fumarates, maleates, gentisates, isethionates, di-p-toluoyltartrates, methane sulphonates, ethanesulphonates, benzenesulphonates, p-toluenesulphonates, cyclohexylsulphamates, quinates, and the like. Base addition salts include those derived from alkali or alkaline earth metal bases or conventional organic bases, such as triethylamine, pyridine, piperidine, morpholine, N-methylmorpholine, and the like. See, for example, “Handbook of Pharmaceutical Salts, Properties, Selection, and Use,” P. H. Stahl and C. G. Wermuch, Eds., ©2008, Wiley-VCH (Zurich, Switzerland), ISBN: 978-3-90639-058-1.

“Spore-specific molecule” refers to any molecule, moiety, or protein that is highly overrepresented in abundance in spores relative to yeast. Conversely, “Yeast-specific molecule” refers to any molecule, moiety, or protein that is highly overrepresented in abundance in yeast relative to yeast. Specifically included in the terms are the proteins identified in Huang M, Hebert A S, Coon J J, Hull C M (2015) “Protein Composition of Infectious Spores Reveals Novel Sexual Development and Germination Factors in Cryptococcus, PLOS Genet 11(8): e1005490 (https://doi.org/10.1371/journal.pgen.1005490). These spore-specific proteins were repeatedly identified by mass spectrometry in spore samples and never in yeast samples and are encoded by the following genes:

TABLE 1
Genes encoding spore-specific proteins.

| | | | |
|---|---|---|---|
| Gene | JEC21 ID | Predicted functions/domains | Deletion phenotype(s) |

Group 1: Replication and Chromosome
Biology

| | | | |
|---|---|---|---|
| TOP1 | CNI03280 | topoisomerase1 | sporulation defects |
| IRR1 | CNA07890 | nuclear cohesion complex component | inviable |

Group 2: Transcription and Splicing

| | | | |
|---|---|---|---|
| RSC9 | CNB00580 | chromatin remodeling complex component | cell fusion defect |
| DST1 | CNF01160 | general transcription elongation factor TFIIS | sporulation defect |
| PRP31 | CNB05520 | U4/U6-U5 snRNP complex component | inviable |
| PRP11 | CND02290 | SF3a splicing factor complex component | inviable |

Group 3: Cellular Transport

| | | | |
|---|---|---|---|
| BCH1 | CNG02530 | specialized cargo export from Golgi | filamentation defect |
| SFH5 | CNE04320 | non-classical phosphatidylinositol transfer | no phenotype |
| | | protein | |
| DDI1 | CNC00460 | vSNARE binding protein | sporulation defect |
| EMC3 | CNF02470 | protein folding in the ER | decreased spore yield |

Group 4: Carbohydrate Metabolism

| | | | |
|---|---|---|---|
| GRE202 | CNG01830 | D-lactaldehyde dehydrogenase | decreased spore yield |
| ISP1^{a} | CNB02490 | conserved in fungi/short chain dehydrogenase | filamentation defect |
| ISP3 | CND04560 | conserved in fungi/mannose-6-phosphate | no phenotype |
| | | isomerase | |
| ISP4 | CNK01510 | conserved in fungi/glycosyl hydrolase | no phenotype |

Group 5: Proteins of Unknown Function

| | | | |
|---|---|---|---|
| ISP2 | CNE01730 | Cryptococcus-specific/no conserved domains | increased sporulation; |
| | | | slow germination |
| ISP5 | CNB04980 | conserved in fungi/ferritin-like superfamily | no phenotype |
| | | domain | |
| ISP6 | CNA04360 | Cryptococcus-specific/transmembrane domain | no phenotype |
| ISP7 | CND00650 | Cryptococcus-specific/no conserved domains | no phenotype |

^{a}Genes encoding proteins with no obvious homologs were named ISP for identified Spore Protein.
dDoi: 10.1371/journal.pgen. 1005490.t003

The spore-specific genes and proteins identified in the above table have the nucleotide and amino acid sequences and protein ID's shown in the Sequence Listing at SEQ. ID. NOS 1-36.

Yeast-specific proteins include, but are not limited to, CND06170, XP_570090.1 (SEQ. ID. NOS. 37 and 38); CND01050, XP_570422.1 (SEQ. ID. NOS. 39 and 40); CNH01340, XP_572322.1 (SEQ. ID. NOS. 41 and 42); CNN02360, XP_568723.1 (SEQ. ID. NOS. 43 and 44); CNB01440, XP_568816.1 (SEQ. ID. NOS. 45 and 46); CNG00410, XP_571739.1 (SEQ. ID. NOS. 47 and 48); CNH02740, XP_572447.1 (SEQ. ID. NOS. 49 and 50); CNJ01750, XP_567350.1 (SEQ. ID. NOS. 51 and 52); CNI02030, XP_572658.1 (SEQ. ID. NOS. 53 and 54); CNB05750, XP_569316.1 (SEQ. ID. NOS. 55 and 56); CNI03560, XP_572607.1 (SEQ. ID. NOS. 57 and 58); CNK01820, XP_567661.1 (SEQ. ID. NOS. 59 and 60); CNI00900, XP_572819.1 (SEQ. ID. NOS. 61 and 62); CNK02880, XP_567883.1 (SEQ. ID. NOS. 63 and 64); CNF00610, XP_571239.1 (SEQ. ID. NOS. 65 and 66); and CNI00870, XP_572850.1 (SEQ. ID. NOS. 66 and 67). These yeast-specific proteins, which are shown in the Sequence Listing, can be utilized as markers of germination.

The gene and encoded protein encoded by CNK01510 (SEQ. ID. NOS. 1 and 2, respectively) is the preferred spore-specific molecule to be labeled in accordance with the assay disclosed herein.

The terms “label,” “marker,” “probe,” “reporter,” and “tag” are used interchangeable and mean a molecular moiety or probe of any structure or configuration, that can be detected by any means, now known or developed in the future, by which a vegetative cell, spore, or molecule bearing such a “label,” “marker,” “probe,” “reporter,” or “tag” can be distinguished from cells, spores, or molecules not bearing such a “label,” “marker,” “probe,” “reporter,” or “tag.” The terms include, without limitation, radioactive labels, fluorescent labels, chromophoric labels, affinity-based labels (such as antibody-type markers), chemiluminescent labels, and the like. Conventional radioactive isotopes used for detection include, without limitation, ³²P, ²H and many others. A huge number of fluorescent and chromophoric probes are known in the art and commercially available from numerous worldwide suppliers, including Life Technologies (Carlsbad, CA, USA), Enzo Life Sciences (Farmingdale, NY, USA), and Sigma-Aldrich (St. Louis, MO, USA). Luciferase is the preferred marker. Complete kits for accomplishing luciferase labeling to a desired substrate are commercially available from several suppliers, including Promega Corporation, Madison, WI (e.g., Promega's NanoLuc®-brand vectors and NanoGlo®-brand luciferase assay systems).

The term “operationally linked” or “operationally connected” when referring to joined polynucleotide sequences denotes that the sequences are in the same reading frame and upstream regulatory sequences will perform as such in relation to downstream structural sequences. Polynucleotide sequences which are operationally linked are not necessarily physically linked directly to one another but may be separated by intervening nucleotides which do not interfere with the operational relationship of the linked sequences. Similarly, when referring to joined polypeptide sequences, operationally linked means that the functionality of the individual joined segments are substantially identical as compared to their functionality prior to being operationally linked. For example, a fluorescent protein or chemiluminescent protein can be fused to a polypeptide of interest and in the fused state retain its fluorescence or chemiluminescence, while the fused polypeptide of interest also retains its original biological activity.

All strains used in the working examples were of the serotype D background (Cryptococcus neoformans var. neoformans strains JEC20 (ATCC 96909) and JEC21 (ATCC 96910 and ATCC MYA-565). Sec Kwon-Chung K J, Edman J C, Wickes B L (1992) “Genetic association of mating types and virulence in Cryptococcus neoformans,” Infect Immun. 60:602-605 (pmid: 1730495) and Moore T D, Edman J C (1993) “The alpha-mating type locus of Cryptococcus neoformans contains a peptide pheromone gene,” Mol Cell Biol. 13:1962-1970 (pmid:8441425). All were handled using standard techniques and media as described in Sherman F. (2002) “Getting started with yeast,” Methods Enzymol. 350:3-41(pmid: 12073320) and Alspaugh J A, Perfect J R, Heitman J. (1998) “Signal transduction pathways regulating differentiation and pathogenicity of Cryptococcus neoformans,” Fungal Genet Biol. 25:1-14 (pmid:9806801).

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made. The indefinite articles “a” and “an” mean “one or more” unless explicitly stated otherwise.

All combinations of method or process steps as used herein can be performed in any order, unless otherwise specified or clearly implied to the contrary by the context in which the referenced combination is made.

The methods disclosed herein can comprise, consist of, or consist essentially of the essential elements and limitations of the method described, as well as any additional or optional ingredients, components, or limitations described herein or otherwise useful in microbiology, biochemistry, and/or mycology.

### The Method:

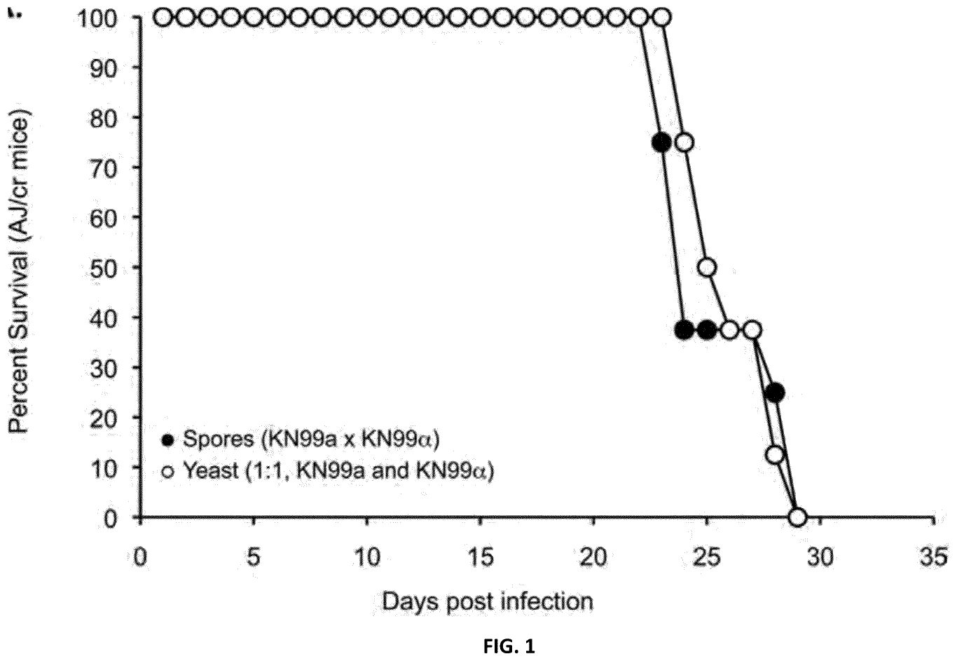

At the core of the present invention is the realization that targeting a cellular process that is specific to organisms that sporulate—namely, spore germination—is likely to yield highly effective antifungal compositions that exhibit fewer side-effects than conventional antifungal drugs when used in humans. (Organisms that produce spores include fungi, bacteria, protists, plant seeds, ferns, and the like.) What then is needed then is a high-throughput assay that can evaluate compounds for their ability to inhibit fungal spore germination. As shown in FIG. 1, it is known that spores are infectious agents. FIG. 1 is a graph showing survivability in a widely accepted mouse model of cryptococcosis. See Giles et al. Infect. Immun. 2009, 77(8):3491. Here, mice were infected with spores or yeast of C. neoformans var. grubii. Spores (10⁵) or yeast (10⁵) were administered to the test animals via intranasal inhalation. Mice infected with spores are shown in black circles; mice infected with yeast are shown in white circles. As can be seen in FIG. 1, the mice died at virtually identical rates. In other words, Cryptococcus spores are just as virulent as the yeast form.

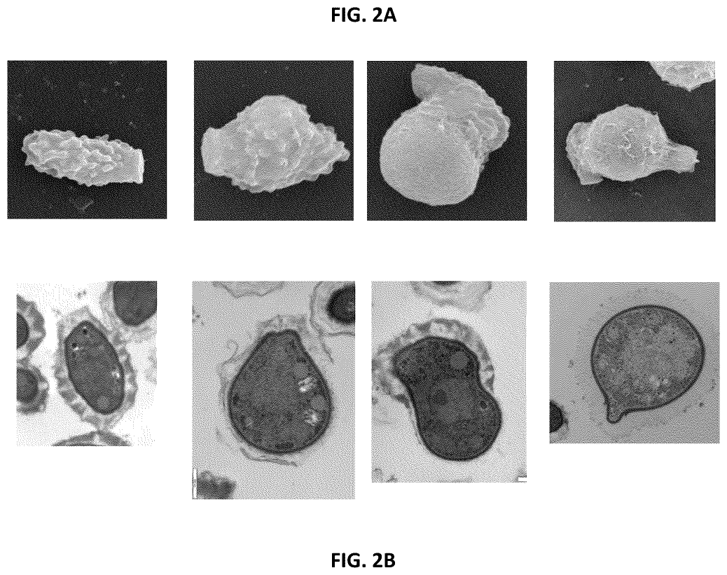

The method functions on two principles. The first principle is that the vegetative form of organisms, especially fungi, are very different, morphologically than their corresponding spores. This is shown quite convincingly in FIGS. 2A and 2B. FIG. 2A is a series of scanning electron micrographs showing the morphological transitions that take place during germination of C. neoformans spores. A C. neoformans spore is shown in the far left photo. The germinating spore is false-colored green. The emerging yeast wall is false-colored yellow. This can been seen initially in the photo second from the left and then in a much more pronounced fashion in the third photo of the series. The daughter cell is false-colored orange and is seen clearly in the far right photo. A simple visual comparison between the far left and far right photos in FIG. 2A illustrates the significant morphological differences between a spore of C. neoformans (on the left) and a yeast (vegetative form, on the right). As can be seen from FIG. 2A, the spore is roughly cylindrical and clearly has a major axis that is much longer than its minor axis. The vegetative yeast form, in contrast is more nearly spherical or globular. Its major and minor axes are much closer in physical length. FIG. 2B shows the same phenomenon using transmission electron microscopy rather than scanning electron microscopy. Spores are quantitatively smaller and more oblong than yeast.

The second principle is that the inventors have identified 18 proteins that are expressed at far greater levels in the spore form as contrasted to the yeast form. Thus, by affixing a marker to one or more of these spore-specific proteins, the extent of germination can be tracked by following changes in the signal generated by the marker as the spore-specific protein is degraded during the germination process.

The first step of the method is to provide bacterial, fungal, or plant spores transformed to contain and express a detectable marker, wherein the marker is operationally linked to a spore-specific or a yeast-specific protein. The marker is preferably a protein fluorophore or protein chemiluminescent marker, such as luciferase, fluorescent protein A, green fluorescent protein, etc. The marker protein is incorporated into spores or yeast by fusing the gene encoding the marker protein to a spore-specific or yeast-specific target gene. The spore then produces the spore-specific protein with the marker attached. (Or the yeast then produces the yeast-specific protein with the marker attached.) The marker will thus generate a first signal associated with the spores. That first signal remains unchanged for as long as the spores remain intact. However, when the spore germinates, the spore-specific protein and its attached marker are degraded, which then alters the signal generated by the attached marker (or the yeast-specific marker is increased). A second signal measurement taken after germination is thus proportional to the extent of germination.

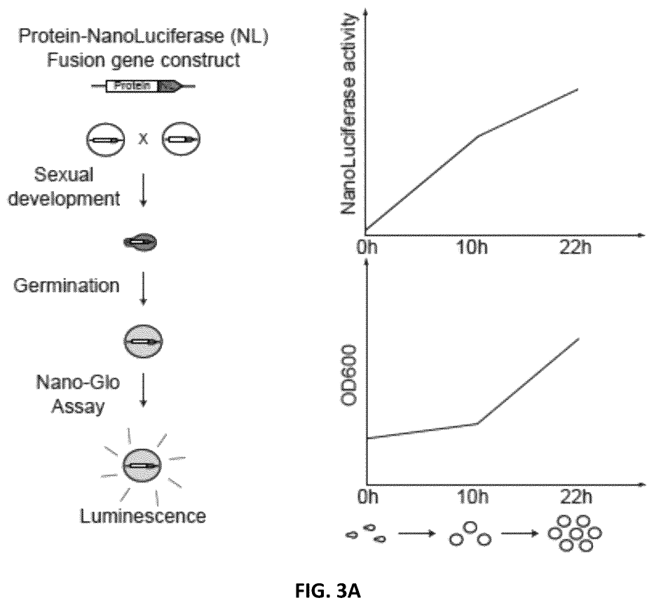

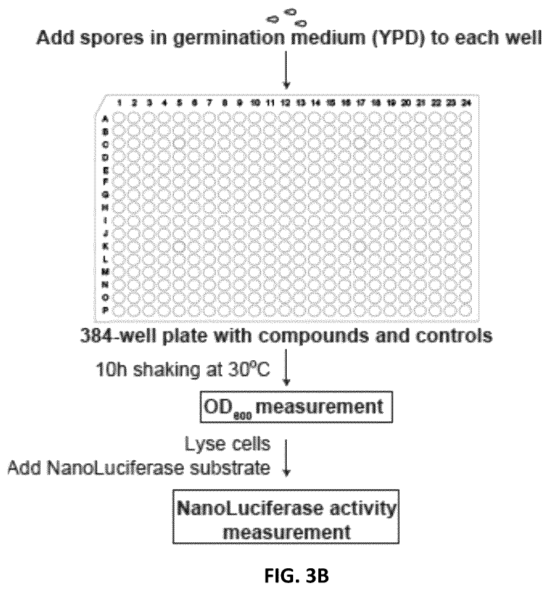

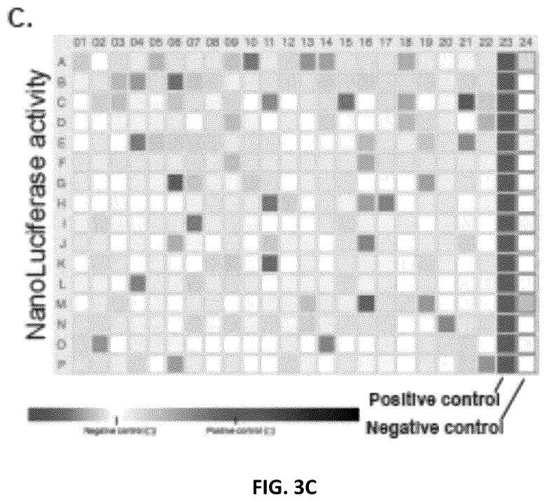

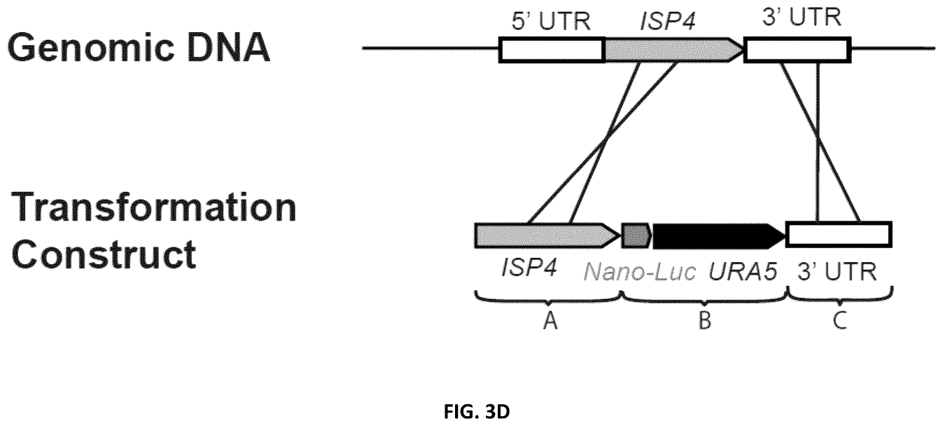

This process is shown schematically in FIG. 3A. As shown in the left-hand side of the figure, the yeast form of the organism (in this case C. neoformans) was transformed to contain a fusion construct comprising a spore-specific protein fused to a luciferase gene. The transformed yeast were cultured to yield a population of propagating yeast that include the fusion construct. The yeast were then induced to sporulate. A first measure of the signal generated by the luciferase marker generated by the fusion construct is taken. This is shown at Time=0 in the two right-hand graphs depicted in FIG. 3A. The upper graph show the signal generated by the reporter as the spore germinate. The lower graph shows the optical density of the culture solution at 600 nm (OD₆₀₀) over the same time period. As can be seen from the two graphs, as the spore germinate and multiply, the optical density increases (as the number of cells increases). In a corresponding fashion, the signal generated by the marker displays a proportional rise. The schematic pictures below show the morphology and number of cells over time.

The assay can be implemented in a massively redundant, massively high-throughput format that is easily automated using conventional multiwall plates and robotic equipment. (Laboratory robotics for handling multiwall culture plates are available from a host of international commercial suppliers, including Agilent Technologies (Santa Clara, California), Beckman Coulter (Grants Pass, Oregon), Hudson Robotics (Springfield, New Jersey), and many others.) For a non-limiting example, see FIG. 3B, which is a schematic diagram showing the workflow for a high-throughput screening assay according to the present disclosure. As shown in FIG. 3B, the method can be implemented using conventional 384-well incubation plates. Spores to be studied are modified to contain a suitable marker, as described earlier. The spores are then incubated in a multiwall plate in a suitable germination medium. For many fungi, yeast extract-peptone-dextrose growth medium (YPD or YEPD) is suitable. (YPD is a well known medium for fungal germination and contains roughly 2% w/v bacto-peptone, 1% w/v yeast extract, and 2% w/v dextrose. A 1 L batch is made by combining 20 g bacto-peptone, 10 g yeast extract, and 20 g dextrose, adding water to 1 L and then autoclaving before use.)

A first signal from each well of the multiwall plate is then taken at the start of the incubation period. The contents of each well can be arranged in any suitably logical fashion, with positive and negative control wells, and wells containing compounds to be tested for their ability to inhibit germination of the spores, perhaps in appropriate serial dilutions of the compounds. The entire multiwall plate is then cultured for a time, temperature, humidity, etc. that is conducive to germination of the spores. After a set time, and OD600 measurement may optionally be taken to confirm that in the control wells the spores responded appropriately. The cells are then lysed, luciferase substrate is added, and a second measurement of the signal generated by the marker is taken. The extent of germination can then be determined by comparing the first signal to the second.

FIG. 3C shows a representative multiwall plate from the resulting from the method just described. Positive and negative control wells are in columns 23 and 24, respectively. Wells that contain germination-inhibiting compounds in various shades of pink/red, with the darker red hues indicating great inhibitory activity. The signals can be gathered, digitized, recorded, and compared using a photomultiplier tube, in conventional fashion. Thus, wells H11, K11, C15, M16, and C21 all appear to contain very effective germination-inhibiting compounds.

FIG. 3D shows a schematic diagram of a vector used to transform a spore so that it includes a marker responsive to germination

An exemplary protocol, using luciferase as the marker, can be accomplished using commercial kits and largely following the manufacture's instructions on how to use the kit. A preferred kit for is Promega's Nano-Luc®-brand vectors and Nano-Glo®-brand luciferase assay system.

Briefly, homologous recombination is utilized to tag spore proteins with luciferase under their endogenous promoters. See FIG. 3D. In this fashion, their expression levels in the spores will remain undisturbed by tagging. As illustrated in FIG. 3D, the transformation construct contains three parts (A, B, and, C). Part A includes the sequence that encodes ISP4 but without a stop codon. Part B includes NanoLuc sequence (GeneBank sequence number KM359770) and C. neoformans URA5 gene (GenBank sequence number AE017347.1), the latter of which serves as a selection marker for cell transformation. Part C includes the 3′ UTR of ISP4, so that together with Part A, the transformation construct will be more favorably integrated into the genome through homologous recombination. Individual parts were generated by regular PCR and the full-length transformation construct was created using fusion PCR. See Davidson R C, Blankenship J R, Kraus P R, de J Berrios M, Hull C M, D'Souza C, et al. A PCR-based strategy to generate integrative targeting alleles with large regions of homology. Microbiology. 2002; 148: 2607-2615. PMID: 12177355. The construct was transformed into cells by biolistic transformation before selection. See Toffaletti D L, Rude T H, Johnston S A, Durack D T, Perfect J R. Gene transfer in Cryptococcus neoformans by use of biolistic delivery of DNA. J Bacteriol. 1993; 175: 1405-1411. PMID: 8444802.

The present inventors have identified a significant number of proteins in C. neoformans that were detected in spores only. Thus, these proteins are all candidates for labelling in the present invention. In C. neoformans and in other fungi where the corresponding genes are conserved, one or more of the following proteins can be labelled with the marker: XP_567740.1 (SEQ. ID. NO: 2), XP_566791.1 (SEQ. ID. NO: 4), XP_570303.1 (SEQ. ID. NO: 6), XP_571089.1 (SEQ. ID. NO: 8), XP_571997.1 (SEQ. ID. NO: 10), XP_569295.1 (SEQ. ID. NO: 12), XP_569173.1 (SEQ. ID. NO: 14), XP_569068.1 (SEQ. ID. NO: 16), XP_569336.1 (SEQ. ID. NO: 18), XP_567136.1 (SEQ. ID. NO: 20), XP_568990.1 (SEQ. ID. NO: 22), XP_570610.1 (SEQ. ID. NO: 24), XP_571921.1 (SEQ. ID. NO: 26), XP_572925.1 (SEQ. ID. NO: 28), XP_570796.1 (SEQ. ID. NO: 30), XP_571548.1 (SEQ. ID. NO: 32), XP_570447.1 (SEQ. ID. NO: 34), XP_571343.1 (SEQ. ID. NO: 36).

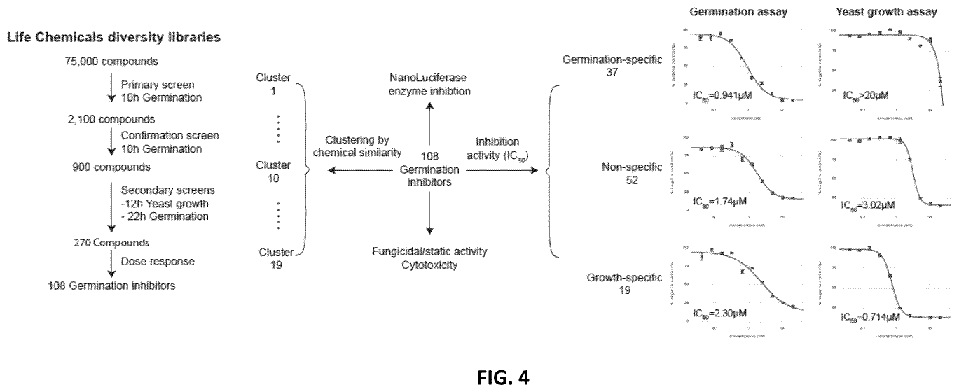

FIG. 4 is a flow chart showing the workflow of a high-throughput screening assay according to the present disclosure. Here, the figure shows how a large library of 75,000 compounds was screened using the present method set up in high-throughput format. As shown on the left-hand side of the figure, the full library was first subjected to a primary screening comprising a 10-hour germination, followed by evaluating which compounds showed initial interested as germination inhibitors. This yielded 2, 100 putative “hits,” i.e., compounds that at least initially showed promise as germination inhibitors. These 2,100 hits were then re-screened and the upper 900 best performing inhibitors were tested further. These 900 compounds were then re-screened using longer germination and yeast growth incubation times. This resulted in 270 compounds being advanced for further study. This group of 270 compounds was then studied using the method described herein to determine if any of the compounds inhibited spore germination and/or fungal growth in a dose-dependent fashion. This final screen yielded 108 compounds from the original 75,000 compounds that inhibited fungal spore germination and/or yeast grown in a dose-dependent fashion.

As shown in the middle panel of FIG. 4, the 108 compounds that were “hits” were then clustered by structural similarity and further tested to see if their anti-fungal properties were germination specific (i.e., primarily germination inhibitory), non-specific, or primarily growth specific. As shown in the right-hand graphs of FIG. 4, 37 of the compounds specifically inhibited germination in a dose-dependent manner; 52 of the compounds were non-specific, dose-dependent inhibitors; and 19 of the compounds specifically inhibited vegetative fungal growth in a dose-dependent manner.

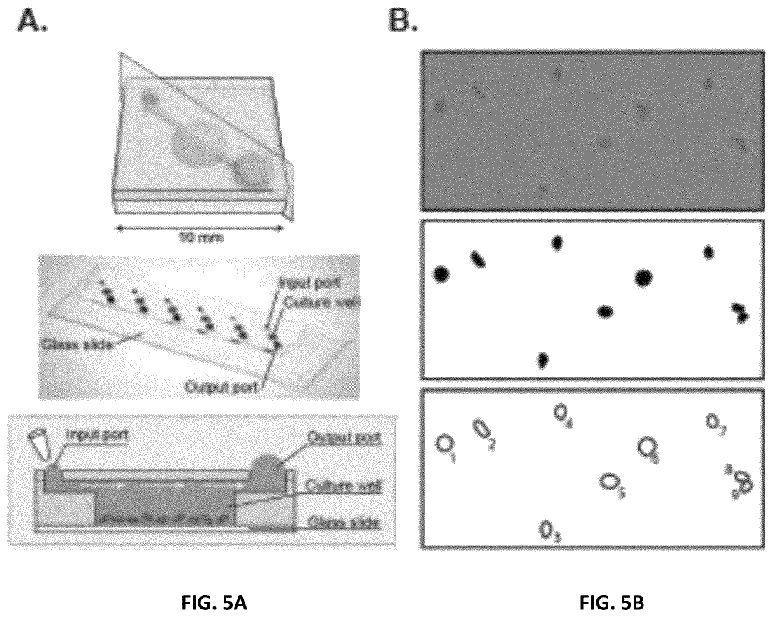

As shown in FIGS. 5A and 5B, the method described herein can also be formatted for continuous studies using a microfluidic test bed. The test bed, depicted schematically in FIG. 5A, comprises a microliter-scale culture well having an input port and an output port. That is, the device includes an input port operationally linked in fluid connection to to a culture well which is operationally linked in fluid connection to an output port. Each microfluidic chamber is built upon a transparent support, such as a glass microscope slide. The microfluidic device is dimensioned and configured to culture and image non-adherent cells, such as spores and germinated fungi, yeast, and the like. The top panel of FIG. 5A shows a perspective view of a single microfluidic culturing device. The middle panel of FIG. 5A shows six (6) such devices disposed on a glass slide. These six devices held 10 μL blue dye. The bottom panel of FIG. 5A shows a front elevation cutaway of the device shown in the top panel. In this bottom panel, fluid flow is depicted as moving from left-to-right. Non-adherent cells are retained within the culture well, while the medium gently flows above them. Compounds to be tested are introduced through the input port, where they then flow to the culture well to interact with the cells therein. When built on an optically transparent substrate, the cells can be visualized and photographed in real time, as shown in the photos in FIG. 5B.

FIG. 5B depicts representative raw images of the fungal cells (spores, germinating cells, and yeast) in the device shown in FIG. 5A. Various imaging processing steps, described in detail below, are then applied to the raw images to discriminate among spores, germinating cells, and yeast. These process steps may include, without limitation, applying a density threshold to the raw images and then automatically detecting and measuring the cells' 2-D area and aspect ratio.

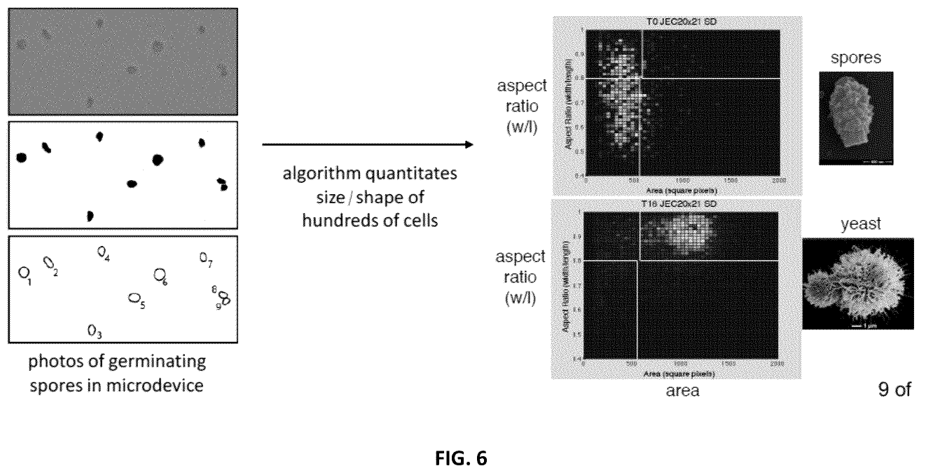

FIG. 6 shows how computer processing can be brought to bear to automatically discriminate between ungerminated spores and vegetative yeast after a culture period has been completed. The left-hand side of FIG. 6 shows the three raw photographs from FIG. 5B. These are raw photographs of the germinating spores in the microfluidic device shown in FIG. 5A. The photos are digitized from the outset. The digitized images were analyzed for the size and shape of each cell in each image. The area of each cell, as well as its aspect ratio can be determined using a public domain, open-source, Java-based image processing program called ImageJ. Several other commercial image processing software packages can also accomplish this task. For example, Stream-brand image analysis software from Olympus Corporation (Waltham, MA) and PAX-it brand image analysis software from MIS, Inc. (Villa Park, Ill.). The images of the cells are then plotted based on their 2-D area (X-axis) versus their aspect ratio (Y-axis) as shown in the right-hand panels of FIG. 6. As can be seen from the plots in FIG. 6, the spores (upper plot) cluster in a distinctly different location and with a distinctly different distribution as compared to the vegetative yeast (lower plot). This is because spores, being more oblong and smaller in area, plot to the left-hand side of the histogram—indicating smaller average area and ovoid nature in the photographs. Spores tend to toward a wider distribution of their aspect ratios and areas. This may be due to the fact that the spores settle in the device at many angles. When photographed, spore aspect ratios and sizes are are more variable than in reality. Yeast, because they are more spherical and larger in area regardless of the position from which they are photographed, plot in a tight cluster in the upper right quadrant of the histograms.

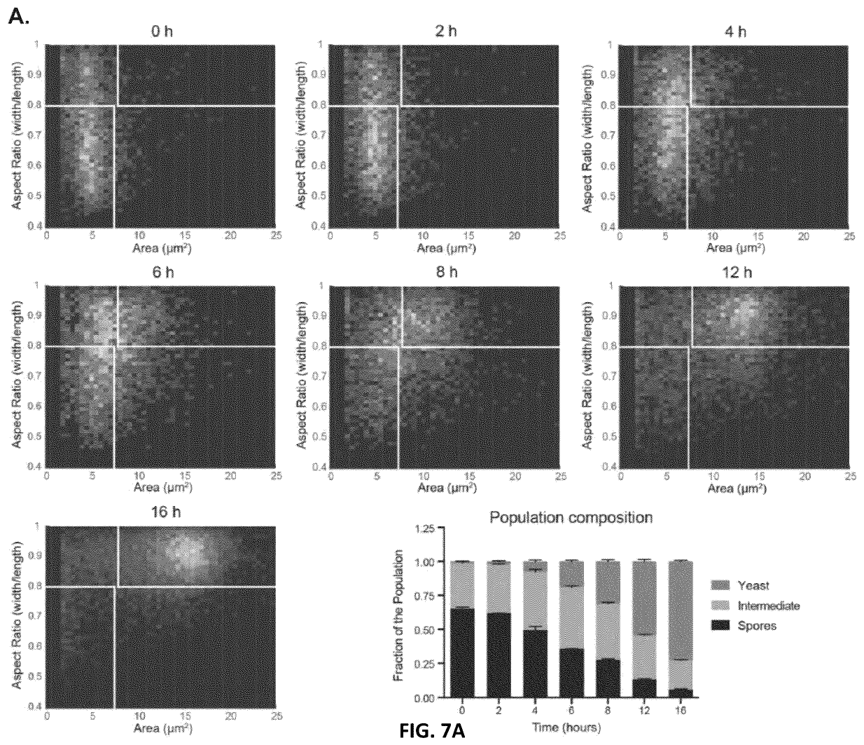

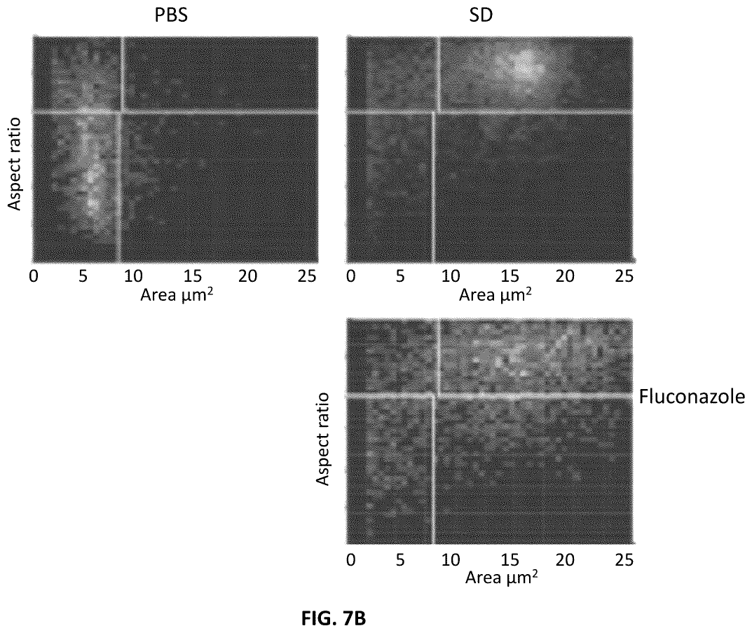

Further examples of how spores, germinating cells, and yeast can be compared is shown in FIGS. 7A and 7B. FIG. 7A depicts a series of photographic analyses further demonstrating that germination in microscale devices as described herein can be determined by cell area versus aspect ratio. Each panel in FIG. 7A depicts the germination dynamics of spores visualized by 2D histograms of cell area vs aspect ratio, as well as a stacked bar plot of the population composition over time (at lower right). Colors are normalized on each plot such that yellow represents the area and aspect ratio combination with the most cells observed and dark blue represents area and aspect ratio combinations that were not observed. Cells in the lower left quadrant are defined as spores; cells in the upper right quadrant as yeast; all remaining cells are classified as intermediates undergoing germination. FIG. 7B shows 2D histograms as in FIG. 7A, but for a 16-hour germination of Cryptococcus spores using PBS as a control (no germination), synthetic dextrose growth medium (SD) alone (full germination in the absence of compounds), and the antifungal compound fluconazole (16 mg/mL) in the presence of growth medium. In this study, we demonstrate that spore germination is a viable target for antifungal development by identifying and characterizing FDA approved drugs able to inhibit both spore germination and yeast replication. These inhibitors have the potential of becoming tools to probe the essential fungal process of spore germination, or repurposed into antifungal therapies. Importantly, we determined that one of the drugs, Pentamidine, was effective at lowering fungal burden in vivo and could be repurposed as a prophylactic treatment against Cryptococcus pathogens.

### Germination Provides a Suitable Target for the Development of Novel Antifungals:

Limited therapies exist to combat fungal disease. Humans and fungi share many biological processes due to their eukaryotic nature. Because fungi-specific drug targets are difficult to find, potent antifungal agents often have toxic side-effects in humans. In the quest to find novel fungal-specific targets, the field has mainly focused on the cell membrane processes (ergosterol biosynthesis), and the fungal cell wall (β(1,3)-glucan synthesis). While these targets have been effective in the discovery of antifungals in the past; the lack of novel antifungal therapies is an indication that these targets currently have limited success. It is critical that novel fungi-specific targets are identified for the development of new antifungals. This requires identifying new cell processes to probe that are unique to fungi. Fungal spore germination provides one of these novel targets.

Fungal spore germination has been previously suggested to be a modified cell cycle. Recently discovered evidence suggests otherwise. In previous studies we identified and characterized spore-enriched proteins. One of these proteins (Isp2) was found to stall germination for two hours prior to initiating vegetative growth. Isp2 showed no apparent phenotype in vegetatively growing yeast. Isp2, along with other spore germination-specific results, indicate that it is unlikely that germination is simply a modified cell cycle. Spore germination in not only a unique fungal process but is also unlike any process defined in humans. The uniqueness of fungal spore germination makes it a prime process to probe in the effort to develop novel antifungals. The examples below show that using germination inhibition as a signal can identify drugs that could be repurposed in the treatment of invasive fungal diseases.

### Targeting Germination Provides a Mechanism for Prevention:

In addition to providing a fungal-specific drug targets, targeting germination provides a unique opportunity for preventing fungal disease. Spores are stress-resistant cell types that are known infectious particles of many fungal pathogens, and have distinct phenotypes compared to yeast when interacting with hosts. Developing antifungals that target all potential infectious particles could be used to protect against fungal pathogens through prophylaxis treatment. If a low toxicity antifungal is found, prophylactic treatment could be administered to immunocompromised individuals, the population most at risk of developing invasive fungal infections.

### Screening Characterized Drugs Allows for the Potential Development of Tools:

The screening of already approved FDA drugs provides a unique opportunity to screen drugs that often have known targets. By screening compounds with known inhibition targets, pathways can be identified that could be potentially important to fungal spore germination. These compounds can be used to probe fungal spore germination to help understand this critical fungal differentiation process. One of the clearest examples of a potential tool in this study was alexidine hydrochloride, which had strong antifungal activity and was a potent inhibitor of fungal spore germination. See the Examples section. This drug has previously been reported to inhibit phospholipases of Cryptococcus (Ganendren et al., 2004). This may suggest that phospholipases are important for viability of fungal spores. The ability of alexidine to inhibit other fungal processes, however, is unclear. In the future, we will use alexidine as a tool to probe the molecular events of phospholipid biosynthesis in fungal spore germination.

### Pentamidine, a Potential Antifungal Prophylactic Against Cryptococcus Infection:

Screening FDA-approved drugs has the benefit of potential repurposing as these drugs could reach patients in need sooner than novel compounds. The Examples section shows that pentamidine has huge promise in repurposing for a variety of reasons. Pentamidine, an antiparasitic, is only approved for use against one fungal pathogen, Pneumocystis. Pentamidine is approved for use in immunocompromised individuals, which is the primary group of individuals infected by Cryptococcus pathogens. Pentamidine already exists in an aerosolized formulation which allows for the drug to build up in the lung, which is the main site where Cryptococcus pathogens establish infections. Finally, this drug is already approved for use prophylactically against Pneumocystis, which would suggest that pentamidine could be used to protect immunocompromised individuals from cryptococcosis.

The Examples section shows that pentamidine was able to inhibit Cryptococcus infectious particles in vitro, was effective at lowering fungal burden in a mouse model of infection and, when used prophylactically, was able to inhibit spore germination in vivo, suggesting that pentamidine can build up in the lung sufficiently to inhibit this stress resistant cell type. The ability to inhibit both cell types, and the nature of this drug, suggest that it could make an ideal prophylactic against Cryptococcus pathogens which cause hundreds of thousands of deaths per year in immunocompromised individuals. While pentamidine is often not the first choice for prophylaxis against Pneumocystis, the data presented herein shows that pentamidine can be used to protect patients against other fungal pathogens generally and Cryptococcus. spp. specifically.

### Pharmaceutical Compositions:

Using the method disclosed herein, the inventors identified four (4) FDA-approved compounds with germination-inhibiting properties that are effective antifungal therapeutics. These four compounds are disulfiram, pentamidine, otilonium bromide, and benzethonium chloride.

Thus, also disclosed herein are pharmaceutical compositions for inhibiting topical and systemic fungal infection in mammals. The compositions comprise a spore germination-inhibiting amount of a compound selected from the group consisting of:

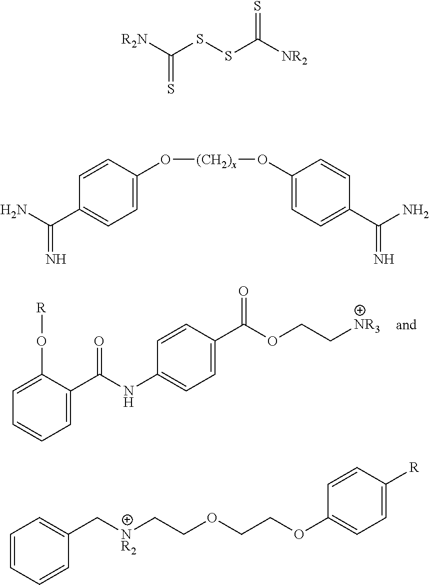

wherein R is linear or branched C₁₋₁₂ alkyl and “x” is an integer of from 1 to 12, and pharmaceutically suitable salts thereof, in combination with a pharmaceutically suitable vehicle.

The active ingredients may be used in combination with a standard, well-known, non-toxic pharmaceutically suitable carrier, adjuvant or vehicle such as, for example, phosphate buffered saline, water, ethanol, polyols, vegetable oils, a wetting agent or an emulsion such as a water/oil emulsion. The composition may be in either a liquid, solid or semi-solid form. For example, the composition may be in the form of a tablet, capsule, ingestible liquid or powder, injectable, suppository, or topical ointment or cream. Proper fluidity can be maintained, for example, by maintaining appropriate particle size in the case of dispersions and by the use of surfactants. It may also be desirable to include isotonic agents, for example, sugars, sodium chloride, and the like. Besides such inert diluents, the composition may also include adjuvants, such as wetting agents, emulsifying and suspending agents, sweetening agents, flavoring agents, perfuming agents, and the like.

Suspensions, in addition to the active compounds, may comprise suspending agents such as, for example, ethoxylated isostearyl alcohols, polyoxyethylene sorbitol and sorbitan esters, microcrystalline cellulose, aluminum metahydroxide, bentonite, agar-agar and tragacanth or mixtures of these substances.

Solid dosage forms such as tablets and capsules can be prepared using techniques well known in the art of pharmacy. For example, compounds as described herein can be tableted with conventional tablet bases such as lactose, sucrose, and cornstarch in combination with binders such as acacia, cornstarch or gelatin, disintegrating agents such as potato starch or alginic acid, and a lubricant such as stearic acid or magnesium stearate. Capsules can be prepared by incorporating these excipients into a gelatin capsule along with antioxidants and the relevant active agent.

For intravenous administration, the compounds may be incorporated into commercial formulations such as Intralipid©-brand fat emulsions for intravenous injection. (“Intralipid” is a registered trademark of Fresenius Kabi AB, Uppsalla, Sweden.) Where desired, the individual components of the formulations may be provided individually, in kit form, for single or multiple use. A typical intravenous dosage of a representative compound as described herein is from about 0.1 mg to 100 mg daily and is preferably from 0.5 mg to 3.0 mg daily. Dosages above and below these stated ranges are specifically within the scope of the claims.

Possible routes of administration of the pharmaceutical compositions include, for example, enteral (e.g., oral and rectal) and parenteral. For example, a liquid preparation may be administered, for example, orally or rectally. Additionally, a homogenous mixture can be completely dispersed in water, admixed under sterile conditions with physiologically acceptable diluents, preservatives, buffers or propellants in order to form a spray or inhalant. The route of administration will, of course, depend upon the desired effect and the medical state of the subject being treated. The dosage of the composition to be administered to the patient may be determined by one of ordinary skill in the art and depends upon various factors such as weight of the patient, age of the patient, immune status of the patient, etc., and is ultimately at the discretion of the medical professional administering the treatment.

With respect to form, the composition may be, for example, a solution, a dispersion, a suspension, an emulsion or a sterile powder which is then reconstituted. The composition may be administered in a single daily dose or multiple doses.

The present disclosure also includes treating fungal infections (topical and systemic) in mammals, including humans, by administering a spore germination-inhibiting amount of one or more compounds described herein. In particular, the compositions of the present invention may be used to treat fungal infections of any and all description.

The above-described pharmaceutical compositions may be utilized in connection with non-human animals, both domestic and non-domestic, as well as humans.

### Examples

The following examples are included to provide a more complete description of the methods and compositions disclosed and claimed herein. The examples are not intended to limit the scope of the claims in any fashion.

### Strain Manipulation, Media and Spore Isolation:

The following strains were used and handled using standard techniques and media as previously described. (Sherman et al., 1987). Cryptococcus neoformans serotype D: JEC20, JEC21, JEC20-GFP, JEC21-GFP (Walsh et al. 2018), serotype A: H99, Candida albicans: SC5314 and Aspergillus fumigatus: AF293. Spores were isolated from cultures as previously described. (Botts et al., 2009). Briefly, yeast of both mating types (JEC20 and JEC21) were grown on YPD for 2 days at 30° C. combined in phosphate buffered saline (PBS) mixed to a 1:1 ratio and spotted onto V8 pH 7 agar plates. Plates were incubated for 5 days at 25° C. and spots were resuspended in 70% Percoll in 1×PBS. Spores were counted using a hemocytometer.

### MIC/MFC Experiments:

All minimum inhibitory concentration (MIC) experiments were based on EUCAST methodology. (European Committee on Antimicrobial Susceptibility Testing, a standards-setting committee of the European Society of Clinical Microbiology and Infectious Diseases; EUCAST Development Laboratory for fungi, Statens Serum Institut, Building 211, Artillerivej 5, DK-2300 Copenhagen, Denmark; www.eucast.org.) Yeast cells were grown overnight in liquid YPD and used to inoculate fresh YPD. After 6-hour incubation, yeast cells were washed in 1×PBS and quantified using a hemocytometer. For each drug, 1.25×10⁵ yeast cells were incubated in RPMI, and 0.33M MOPS, PH 7 at varying concentrations of inhibitors, with a final volume of 200 μL. Cryptococcus neoformans cells were incubated for 2 days at 30° C. while Candida albicans strains were incubated for 2 days at 35° C. OD₆₀₀ readings were used to assess the MIC values for each drug. To determine minimum fungicidal concentrations (MFC) values, 3 L per well were plated on YPD and allowed to grow for 2 days. Spinning down of 96-well plates and washing did not alter the read outs of the MFC experiment.

For Aspergillus fumigatus MIC, conidia were collected using 0.01% Tween 80 in PBS after 3 days of growth on glucose medium media plates. Conidia at a final concentration of 2×10⁴ cells were incubated in RPMI, 0.33 M MOPS, and 2% glucose at pH 7 at varying concentrations of inhibitors, with a final volume of 200 μL. MIC values were assessed based on the lowest concentration of drug that had complete absence of germ tubes or hyphae.

### Quantitative Germination Assay:

All germination assays are based on Barkal et al., 2016. Briefly, microfluidic devices were loaded with 1×10⁵ spores, and at 0 hours, SD media with drug of interest, were added to the sample. Spores were allowed to germinated at 30° C. in a humidified chamber and cells were monitored every two (2) hours for 16 hours. Each assay was performed in two (2) individual wells with three (3) field of views acquired from each well. All images were analyzed as previously described based on cell shape and size. Population ratio of spores, intermediate, and yeast cells were determined. Error bars in plots are based on variation between all fields of view acquired. All experiments were able to be reproduced independently. After the 16-hour experiment, samples were plated on YPD and allowed to grow at 30° C. to determine if drugs were completely germicidal or not based on lack of growth. If assays were unable to be performed in microfluidic devices, the 2×10⁵ spores were incubated in identical conditions outside of PDMS devices and only loaded into devices for image acquisition.

### Fungal Burden Animal Studies:

All yeast cells were cultured overnight in YPD, washed and diluted to 5×10⁶ cells. For JEC20 and JEC21, 2.5×10⁶ cells of each were combined. Spores were cultured as previously described and diluted to 2×10⁶ cells. All experiments were performed on 8- to 10-week old C57BL/6J (Jackson Laboratory, Bar Harbor, Maine, USA) female mice (5 mice per group). All mice were infected intranasally with a total of 50 μL. All dosing was performed with 4 mg/kg/day or 1×PBS for three (3) days either prior to infection or 1-day post-infection. Mice were sacked day-4 post-infection and lungs were collected, processed, and fungal burden was assessed.

### In Vivo Germination:

Female mice, 8- to 10-week-old C57BL/6J (Jackson Laboratory) female mice (3 mice per group) were used. Mice were dosed with either 4 mg/kg/day or 1×PBS (50 μL) for three (3) consecutive days. Mice were intranasally infected with 2×10⁶ JEC20-GFP×JEC21-GFP spores, strains described in Walsh et al., 2018. After 8 hours post-infection, mice were sacked and lavaged with 0.05% TirtonX in 1×PBS. Lavage suspension underwent a series of treatments and washes, in order: red blood cell lysis (ACK lysing buffer, 2 mL, 5 minutes), formaldehyde fixation (4%, 500 μL, 30 minutes) and calcofluor white staining (25 μg/mL, 20 μL for 1 minute). Cells (50-100 per mouse) were imaged, and identified as Cryptococcus neoformans cells based on green fluorescent signal or cyan staining from calcofluor staining. Cells surface area and aspect ratio were measured in ImageJ and cells were classified as spores, intermediates, or yeast based on size and shapes parameters used in the quantitative germination assay.

### Identifying Inhibitors of Germination and Growth

To identify inhibitors of Cryptococcus neoformans spore germination, a high throughput screen was developed that utilizes a nanoluciferase construct to monitor whether spores germinate in the presence of inhibitor. Briefly a protein luciferase construct was created resulting in a low luciferase signal for non-germinated spores and a high signal from germinated and replicating cells. The screen was coupled with OD₆₀₀ readings to monitor the ability of compounds to inhibit yeast replication.

The examples focused on FDA-approved drugs, as these drugs have the potential of being repurposed into antifungal therapeutics. To determine whether any FDA-approved drugs were able to inhibit Cryptococcus neoformans spore germination and yeast replication, the aforementioned high throughput screen was performed on the L1300 Selleck FDA-Approved Drug Library containing an array of 1108 compounds. This library of compounds is available commercially from Selleck Chemicals, 14408 W Sylvanfield Drive, Houston, TX 77014, USA.

The screening was successful at identifying known antifungal drugs as inhibitors of yeast replication as indicated by an OD₆₀₀ signal of less than 75% of the negative control (Table 2). For the purpose of these examples, antifungal drugs were defined as any FDA-approved drug used in the treatment of fungal infections. Of these 23 known antifungal drugs, only six (6) were identified as inhibitors of spore germination, indicated by a luciferase signal of less than 30% of the negative control. These germination inhibitors demonstrated normal nanoluciferase signal dose response curves (data not shown).

TABLE 2
Antifungal drugs used to treat fungal infections and their ability to
inhibit Cryptococcus neoformans spore germination (based on
luciferase signal) and yeast replication (based on OD₆₀₀).

| | | Germination | Replication |
|---|---|---|---|
| | | Percent | Percent |
| | Drugs | Luciferase Signal | OD₆₀₀ |
| Inhibitors of | Pentamidine HCl | 6.5 | 38.3 |
| Germination (6) | Bifonazole | 13.6 | 33.4 |
| Less than 30% | Econazole nitrate | 16.1 | 33.1 |
| Luciferase Signal | Isoconazole nitrate | 16.8 | 37.0 |
| | Tioconazoie | 25.0 | 36.8 |
| | Miconazole nitrate | 25.5 | 38.2 |
| Non-inhibitors of | Butoconazole | 41.5 | 33.4 |
| Germination (17) | nitrate | | |
| | Fenticonazole | 49.1 | 36.4 |
| | nitrate | | |
| | Naftifine HCl | 55.5 | 34.5 |
| | Sulconazole nitrate | 57.8 | 40.9 |
| | Butenafine HCl | 57.5 | 32.9 |
| | Tolnaftate | 60.2 | 47.3 |
| | Liranaftate | 64.8 | 37.6 |
| | Clotrimazole | 65.9 | 34.1 |
| | Fluconazole | 84.0 | 72.2 |
| | Amphotericin B | 84.6 | 45.7 |
| | Amorolfine HCl | 88.6 | 41.4 |
| | Caspofungin acetate | 89.7 | 48.5 |
| | Climbazole | 151.5 | 38.9 |
| | Ketoconazole | 154.8 | 35.4 |
| | Itraconazole | 159.6 | 58.9 |
| | Posaconazole | 167.2 | 46.7 |
| | Voriconazole | 173.9 | 35.8 |

In addition to the antifungal drugs from the screen, 60 other inhibitors of yeast replication were identified, 16 of which were also inhibitors of spore germination (Table 3). These inhibitors have a wide range of clinical functions, including quaternary ammonium compounds (“QACs”) and mammalian target of rapamycin (“mTOR” inhibitors (i.e.,) which are known to have broad effects on eukaryotic processes. Some drugs used in treating neurological diseases were also identified. Finally, antimicrobial and antihelminth drugs were also identified to inhibit germination. All compounds, with the exception of doxercalciferol, demonstrated appropriate nanoluciferase dose response curves (data not shown). Only a handful of compounds were pursued further in the examples due to limited availability of certain drugs. Representatives from each group, however, were selected for further characterization. Finally, five inhibitors of only germination were identified (see below).

TABLE 3
FDA-approved drugs able to inhibit spore germination and yeast replication. List
of drugs, their ability to inhibit Cryptococcus neoformans spore germination
(based on luciferase signal) and yeast replication (based on OD600), as well as
their function as listed by L1300 Selleck FDA Approved Drug Library.

| | | Germination | | |
|---|---|---|---|---|
| | | Percent | | |
| | | Luciferase | Replication | |
| | | Signal | Percent OD₆₀₀ | Function |
| Germination | Cetylpyridinium | 4.2 | 31.9 | Infection |
| and | chloride | | | |
| Growth | Domiphen bromide | 4.4 | 63.9 | Infection |
| Inhibitors | Cetrimonium | 4.4 | 63.2 | Infection |
| | bromide | | | |
| | Alexidine Hc1 | 4.6 | 29.6 | |
| | Otilonium bromide | 6.9 | 29.0 | Cardiovascular |
| | | | | Disease |
| | Benzethonium | 6.9 | 30.3 | Neurological |
| | chloride | | | Disease |
| | Niclosamide | 7.8 | 43.4 | |
| | PCl-32765 | 10.6 | 70.3 | Neurological |
| | | | | Disease |
| | Everolimus | 15.7 | 67.7 | Cancer |
| | Doxercalciferol | 17.1 | 55.2 | Endocrinology |
| | Rapamycin | 18.5 | 61.8 | Immunology |
| | Temsirolimus | 21.3 | 59.2 | Cancer |
| | Ezetimibe | 22.0 | 51.3 | Cardiovascular |
| | | | | Disease |
| | Dequalinium | 22.3 | 47.9 | |
| | chloride | | | |
| | Disulfiram | 22.7 | 65.6 | Neurological |
| | | | | Disease |
| | Biperiden HCl | 23.4 | 56.6 | Neurological |
| | | | | Disease |

Together these results give a set of compounds that are germination inhibitors and replication inhibitors that can be further investigated as potential targets for repurposing or to elucidate germination processes. Inhibitors of both germination and yeast replication were prioritized for further study.

### Antifungal Drugs are Inhibitors of Fungal Pathogen Vegetative Growth:

To confirm the ability of the known antifungals to inhibit yeast replication, minimum inhibitory concentration (MIC) and minimum fungicidal concentration (MFC) testing was performed on the top three germination inhibition hits. All three antifungal compounds inhibited replication of Cryptococcus neoformans yeast of both serotype A and D, while being less potent against Candida albicans (Table 4). All of the antifungal drugs were fungicidal with the exception of bifonazole against H99.

TABLE 4
Ability to inhibit fungal pathogens of antifungal drug germination-
inhibitor hits. MIC/MFC values of top three germination inhibitors
against prominent human fungal pathogens.

| | Cryptococcus | | | |
|---|---|---|---|---|
| | neoformans | | Cryptococcus | |
| | (JEC21) | | neoformans (H99) | |
| | MIC | MFC | MIC | |
| | (μg/mL) | (μg/mL) | (μg/mL) | MFC (μg/mL) |
| Pentamidine isethionate | 1.56 | 3.13 | 6.25 | 6.25 |
| Bifonazole | 6.25 | 6.25 | 6.25 | >100 |
| Econazole nitrate | <0.78 | 6.25 | <0.78 | 6.25 |

| | | | Aspergillus |
|---|---|---|---|
| | Candida albicans (SC5314) | | Fumigatus (AF293) |
| | MIC (μg/mL) | MFC (μg/mL) | MIC (μg/mL) |
| Pentamidine | 50 | 50 | >100 |
| isethionate | | | |
| Bifonazole | >100 | >100 | >100 |
| Econazole nitrate | 6.25 | 12.5 | 3.13 |

Pentamidine and bifonazole were unable to inhibit Aspergillus fumigatus while econazole nitrate was able to inhibit its growth. It is important to note the Aspergillus fumigatus inhibition testing is performed on conidia, their asexual spore (Table 4). Together these results confirm the ability of these antifungals to inhibit fungal growth in a fungicidal manner.

### Antifungal Drugs are Inhibitors of Fungal Spore Germination:

Once yeast replication inhibition was confirmed, the ability of the drugs to inhibit spore germination was characterized using a quantified microfluidics-based germination assay where the changes in size and morphology are monitored as small ovoid spores germinate into large circular yeast.

Pentamidine isethionate was able to successfully inhibit spore germination as seen by a decrease in morphology transition (data not shown). While germination is not completely halted, the spores were only able to circularize partially and unable to transition into the yeast state. It is important to note that all of the spores were inhibited, indicating that none of the ˜10,000 spores showed inherent resistance and escape from inhibition. Due to the hydrophobic nature of bifonazole and econazole nitrate, the PDMS devices resulted in sequestration of the compounds and the assays could not be performed in the microfluidic devices. To determine if these compounds had an effect on spore germination, the assay was performed outside of the microfluidic device and imaged at 0 and 16 hours. Both econazole nitrate and bifonazole were able to inhibit spore germination effectively with spore escape apparent in bifonazole-treated spores as determined by a yeast population increase. None of these drugs were fully germicidal at these concentrations. These assays confirm that the high throughput screen identified antifungal drugs that are potent inhibitors of spore germination.

### FDA Drug Hits are Inhibitors of Fungal Pathogen Vegetative Growth:

To determine the ability of the 16 non-antifungal drugs to inhibit yeast growth, MIC and MFC testing was performed on nine of the 16 drugs. The nine drugs were selected based on dose response curves, drug availability and ensuring that all classes of inhibitors were tested. Seven inhibitors were able to inhibit yeast replication to varying degrees (Table 5) while biperiden HCl and ezetimibe, were unable to inhibit yeast growth (data not shown). All drugs were tested against Aspergillus fumigatus with varying degrees of success. Notably alexidine was extremely potent against A. fumigatus. Additionally, cetylpyridinium bromide, otilonium bromide, benzethonium chloride and disulfiram were all able to inhibit A. fumigatus. (Table 5)

TABLE 5
Ability to inhibit fungal pathogens of FDA drugs germination-
inhibitor hits. MIC/MFC values of germination inhibitors against
prominent human fungal pathogens.

| | Cryptococcus | | | |
|---|---|---|---|---|
| | neoformans | | Cryptococcus | |
| | (JEC21) | | neoformans (H99) | |
| | MIC | | MIC | |
| | (μg/mL) | MFC (μg/mL) | (μg/mL) | MFC (μg/mL) |
| Cetylpyridinium | <0.78 | <0.78 | <0.78 | <0.78 |
| chloride | | | | |
| Alexidine HCl | <0.78 | <0.78 | <0.78 | <0.78 |
| Otilonium bromide | 3.13 | 3.13 | 3.13 | 3.13 |
| Benzelthonium | 3.13 | 3.13 | 3.13 | 3.13 |
| chloride | | | | |
| Niclosamide | <0.78 | 1.56 | 1.56 | >100 |
| Temsirolimus | 6.25 | 6.25 | 6.25 | 6.25 |
| Disulfiram | 3.13 | 3.13 | 6.25 | 6.25 |

| | | | Aspergillus |
|---|---|---|---|
| | | | Fumigatus |
| | Candida albicans (SC5314) | | (AF293) |
| | MIC (μg/mL) | MFC (μg/mL) | MIC (μg/mL) |
| Cetylpyridinium | 1.56 | 3.13 | 1.56 |
| chloride | | | |
| Alexidine HCl | <0.78 | <0.78 | <0.78 |
| Otilonium bromide | 3.13 | 3.13 | 6.25 |
| Benzelthonium | 6.25 | 12.5 | 12.5 |
| chloride | | | |
| Niclosamide | >100 | >100 | >100 |
| Temsirolimus | 1.56 | 1.56 | >100 |
| Disulfiram | 6.25 | 12.5 | 25 |

These results indicate that these FDA-approved drugs have the ability to inhibit fungal pathogen vegetative growth and kill fungal cells. While some of these drugs have previously been shown to have antifungal activities, some have not.

### FDA Drug Hits are Inhibitors of Fungal Spore Germination:

To determine the ability of these seven drugs, which inhibit fungal vegetative growth, to inhibit spore germination; germination assays were performed on the drugs at a concentration of 25 μg/mL. All seven of these drugs were able to inhibit germination to different extents (data not shown).

Five of the seven drugs were tested in microfluidic devices. Alexidine hydrochloride, an antimicrobial, and otilonium bromide, an antimuscarinic used to treat irritable bowel syndrome, were both able to completely inhibit spore germination, as seen by the lack of change in morphology. Both of these drugs were fully germicidal. Niclosamide, an antihelminth that inhibits oxidative phosphorylation, was also able to completely inhibit germination, but was not fully germicidal. Temsirolimus, an mTOR inhibitor used in some cancer treatments, was able to partially inhibit germination and appeared to stall germination strongly between 6 and 8 hours. When spores were exposed to temsirolimus they were able to circularize but appeared to have difficulty growing in size. Finally, disulfiram, an alcohol dehydrogenase inhibitor used in the treatment of alcoholism, was a weak inhibitor of germination leading to about a 2-hour stall in germination overall at this concentration. At higher concentrations, a similar stall to that observed with temsirolimus was observed (data not shown). Neither temsirolimus nor disulfiram were germicidal.

Cetylpyridinium chloride and benzethonium chloride, both quaternary ammonium salts, were unable to be tested in the microfluidic devices due to their viscosity and were therefore tested in outside the devices and imaged at 0 and 16 hours. Both drugs were able to inhibit spore germination completely and were fully germicidal at this concentration. These assays confirm that the method disclosed herein has utility to identify a variety of non-antifungal, FDA-approved drugs that are able to inhibit fungal spore germination to varying degrees. These results also start to elucidate potential molecular processes crucial for fungal spore germination.

### Pentamidine Ubiquitously Slows Germination:

Pentamidine was selected for further study due to many factors that make it a good candidate for repurposing. A range of concentrations of pentamidine isethionate was tested in a germination assay. As concentrations of pentamidine increased, spore germination became slower. However, no individual spores were able to escape inhibition, as seen by the lack of spores in the yeast state at higher concentrations. While pentamidine was not germicidal at lower concentration, at 50 μg/mL pentamidine showed germicidal activity. These results suggest that pentamidine slows the germination of spores ubiquitously and at high enough concentrations is sporicidal.

### Pentamidine Treatment Lowers Fungal Burden in Mouse Lung:

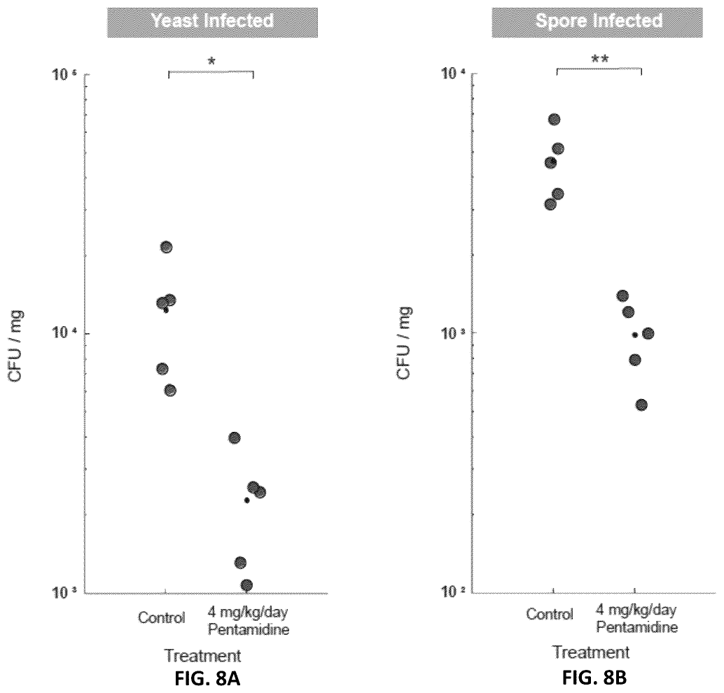

Pentamidine is a successful inhibitor of Cryptococcus neoformans yeast replication in vitro. For repurposing potential, it is important to determine drug efficacy in vivo. For this purpose, the ability of pentamidine to lower the fungal burden in mouse lungs infected by both spores and yeast was determined. One-day post-infection intranasal dosing was begun at 4 mg/kg/day and the mice were treated for three consecutive days. On the fourth day post-infection, lungs were collected and fungal burden was determined. Pentamidine-treated mice had significantly lower fungal burdens in the lung than PBS-treated mice, in both yeast- and spore-infected mice. See FIG. 8A and FIG. 8B, respectively. These results indicate that pentamidine is able to inhibit yeast replication in vivo.

### Prophylactic Pentamidine Inhibits Spore Germination In Vivo:

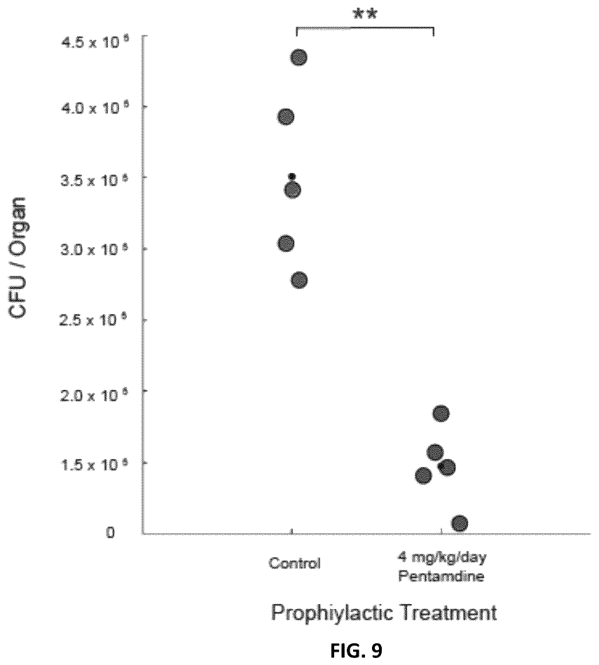

Pentamidine is a successful inhibitor of spore germination in vitro. It is important, though, to determine drug efficacy in vivo. Therefore, the ability of pentamidine to inhibit germination of spores in mouse lungs was determined. To determine if prophylactic pentamidine had an effect on fungal lung burden, mice were treated with 4 mg/kg/day of pentamidine or 1×PBS for three consecutive days. After three days of infection, mice were infected with JEC20×JEC21 spores and 4-days post infection, mouse lungs were collected and lung fungal burden was determined. The results are shown in FIG. 9. As evidenced by data in FIG. 9, pentamidine prophylaxis was successful in decreasing spore-mediated lung burden. These results indicate that spore germination was inhibited in vivo.

In vivo spore germination has never been characterized mainly due to technical hurdles. Using a novel assay, Cryptococcus neoformans cells were recovered from prophylactically treated, spore-infected mouse lungs 8 hours post infection. This was an early enough time point where no budding yeast were recovered from mouse lungs, ensuring that all cells were spore derived and not budding derived. Based on size and shape of the cells, the level of in vivo spore germination was quantified. Prophylactic pentamidine was able to inhibit spore germination as indicated by a higher spore percent and a lower yeast percent in pentamidine-treated mice. Together these results demonstrate that prophylactic pentamidine has in vivo activity against Cryptococcus neoformans spores, indicating it is useful to prophylactically treat (i.e., prevent) fungal infection.

What is claimed is:

1. A method of treating fungal infections in mammals, the method comprising administering a spore germination-inhibiting concentration of a compound, and salts thereof; wherein the compound is selected by:
(a) providing bacterial, fungal, or plant spores transformed to contain and express a detectable marker, wherein the marker when expressed, is operationally linked to a spore-specific or yeast-specific protein, in a medium and under environmental conditions in which the spores will germinate, and measuring a first signal output generated by the marker prior to the spores initiating germination;
(b) contacting the spores of step (a) with the compound;
(c) incubating the spores of step (b) under environmental conditions and for a time wherein spores not treated with the compound will germinate; and
(d) determining extent of germination of spores by measuring a second signal output generated by the marker, wherein a difference between the first signal output and the second signal output is proportional to the extent of germination of the spores.

2. The method of claim 1, wherein the marker is operationally linked to a gene encoding a spore-specific protein selected from the group consisting of XP_567740.1 (SEQ. ID. NO: 2), XP_566791.1 (SEQ. ID. NO: 4), XP_570303.1 (SEQ. ID. NO: 6), XP_571089.1 (SEQ. ID. NO: 8), XP_571997.1 (SEQ. ID. NO: 10), XP_569295.1 (SEQ. ID. NO: 12), XP_569173.1 (SEQ. ID. NO: 14), XP_569068.1 (SEQ. ID. NO: 16), XP_569336.1 (SEQ. ID. NO: 18), XP_567136.1 (SEQ. ID. NO: 20), XP_568990.1 (SEQ. ID. NO: 22), XP_570610.1 (SEQ. ID. NO: 24), XP_571921.1 (SEQ. ID. NO: 26), XP_572925.1 (SEQ. ID. NO: 28), XP_570796.1 (SEQ. ID. NO: 30), XP_571548.1 (SEQ. ID. NO: 32), XP_570447.1 (SEQ. ID. NO: 34), and XP_571343.1 (SEQ. ID. NO: 36).

3. The method of claim 1, wherein the marker when expressed, is operationally linked to a gene selected from the group consisting of SEQ. ID. NOS: 1, 3, 5, 7, 9, 11, 13, 15, 17, 19, 21, 23, 25, 27, 29, 30, 31, 33, and 35.

4. The method of claim 1, wherein the marker, when expressed, is operationally linked to a protein encoded by gene CNK01510 (SEQ. ID. NO: 1).

5. The method of claim 1, wherein the spores of step (a) are of a genus selected from the group consisting of Histoplasma, Blastomyces, Aspergillus, Coccidioides, Sporothrix, Penicillium, and Cryptococcus.

6. The method of claim 1, wherein the spores of step (a) are a species selected from the group consisting of Histoplasma capsulatum, Blastomyces dermatitidis, Aspergillus fumigatus, Coccidioides immitis, Sporothrix schenkii, Penicillium marneffei, and Cryptococcus neoformans.

7. The method of claim 1, wherein the detectable marker, when expressed, is a fluorophore or a chemiluminescent marker.

8. The method of claim 1, wherein the detectable marker, when expressed, is luciferase.

9. The method of claim 7, wherein the marker when expressed, is operationally linked to a gene encoding a spore-specific protein selected from the group consisting of XP_567740.1 (SEQ. ID. NO: 2), XP_566791.1 (SEQ. ID. NO: 4), XP_570303.1 (SEQ. ID. NO: 6), XP_571089.1 (SEQ. ID. NO: 8), XP_571997.1 (SEQ. ID. NO: 10), XP_569295.1 (SEQ. ID. NO: 12), XP_569173.1 (SEQ. ID. NO: 14), XP_569068.1 (SEQ. ID. NO: 16), XP_569336.1 (SEQ. ID. NO: 18), XP_567136.1 (SEQ. ID. NO: 20), XP_568990.1 (SEQ. ID. NO: 22), XP_570610.1 (SEQ. ID. NO: 24), XP_571921.1 (SEQ. ID. NO: 26), XP_572925.1 (SEQ. ID. NO: 28), XP_570796.1 (SEQ. ID. NO: 30), XP_571548.1 (SEQ. ID. NO: 32), XP_570447.1 (SEQ. ID. NO: 34), and XP_571343.1 (SEQ. ID. NO: 36).

10. The method of claim 7, wherein the marker when expressed, is operationally linked to a gene selected from the group consisting of SEQ. ID. NOS: 1, 3, 5, 7, 9, 11, 13, 15, 17, 19, 21, 23, 25, 27, 29, 30, 31, 33, and 35.

11. The method of claim 7, wherein the marker when expressed, is operationally linked to a protein encoded by gene CNK01510 (SEQ. ID. NO: 1).

12. The method of claim 7, wherein the spores of step (a) are of a genus selected from the group consisting of Histoplasma, Blastomyces, Aspergillus, Coccidioides, Sporothrix, Penicillium, and Cryptococcus.

13. The method of claim 7, wherein the spores of step (a) are a species selected from the group consisting of Histoplasma capsulatum, Blastomyces dermatitidis, Aspergillus fumigatus, Coccidioides immitis, Sporothrix schenkii, Penicillium marneffei, and Cryptococcus neoformans.

14. The method of claim 1, wherein the difference between the first signal output and the second signal output of the spores treated with the compound is less than 30% of that of the spores not treated with the compound.

15. The method of claim 1, wherein the compound is selected from the group consisting of 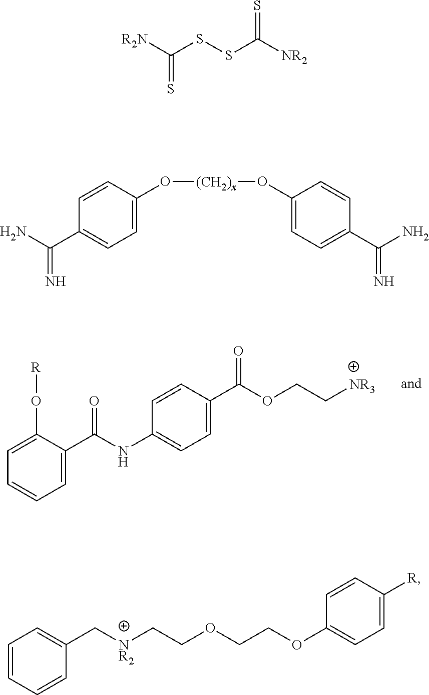
wherein R is linker or branched C₁₋₁₂ alkyl and “x” is an integer of from 1 to 12.

16. The method of claim 1, wherein the compound is: 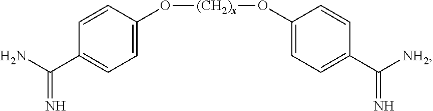
wherein “x” is an integer of from 1 to 12.
